SEQUENCE LISTING (57 sequences)

SEQ ID NO: 1 - protein, 15 aa, Artificial
  CRISPR/Cas motif
IGLDIGTNSVGWAVI

SEQ ID NO: 2 - protein, 8 aa, Artificial
  CRISPR/Cas motif
IVIEMARE

SEQ ID NO: 3 - protein, 27 aa, Artificial
  CRISPR/Cas motif
DVDHIVPQSFLKDDSIDNKVLTRSDKN

SEQ ID NO: 4 - protein, 8 aa, Artificial
  CRISPR/Cas motif
HHAHDAYL

SEQ ID NO: 5 - DNA, 82 bp, Artificial
  DNA encoding CRISPR guide RNA
gttttagagctagaaatagcaagttaaaataaggctagtccgttatcaacttgaaaaagtggcaccgagtcggtgctttttt

SEQ ID NO: 6 - RNA, 82 nt, Artificial
  CRISPR guide RNA
guuuuagagcuagaaauagcaaguuaaaauaaggcuaguccguuaucaacuugaaaaaguggcaccgagucggugcuuuuuu

SEQ ID NO: 7 - RNA, 19 nt, Artificial
  CRISPR guide RNA
aauuucuacuguuguagau

SEQ ID NO: 8 - RNA, 19 nt, Artificial
  CRISPR guide RNA
aauuucugcuguugcagau

SEQ ID NO: 9 - RNA, 19 nt, Artificial
  CRISPR guide RNA
aauuuccacuguuguggau

SEQ ID NO: 10 - RNA, 19 nt, Artificial
  CRISPR guide RNA
aauuccuacuguuguaggu

SEQ ID NO: 11 - RNA, 19 nt, Artificial
  CRISPR guide RNA
aauuucuacuauuguagau

SEQ ID NO: 12 - RNA, 19 nt, Artificial
  CRISPR guide RNA
aauuucuacugcuguagau

SEQ ID NO: 13 - RNA, 18 nt, Artificial
  CRISPR guide RNA
aauuucuacuuuguagau

SEQ ID NO: 14 - RNA, 17 nt, Artificial
  CRISPR guide RNA
aauuucuacuuguagau

SEQ ID NO: 15 - RNA, 67 nt, Artificial
  CRISPR guide RNA
gaauuuuucaacgggugugccaauggccacuuuccagguggcaaagcccguugagcuucucaaaaag

SEQ ID NO: 16 - RNA, 79 nt, Artificial
  CRISPR guide RNA
gucuagaggacagaauuuuucaacgggugugccaauggccacuuuccagguggcaaagcccguugagcuucucaaaaag

SEQ ID NO: 17 - RNA, 78 nt, Artificial
  CRISPR guide RNA
ucuagaggacagaauuuuucaacgggugugccaauggccacuuuccagguggcaaagcccguugagcuucucaaaaag

SEQ ID NO: 18 - RNA, 36 nt, Artificial
  CRISPR guide RNA
acuuuccaggcaaagcccguugagcuucucaaaaag

SEQ ID NO: 19 - RNA, 9 nt, Artificial
  CRISPR guide RNA
agcuucuca

SEQ ID NO: 20 - RNA, 8 nt, Artificial
  CRISPR guide RNA
gcuucuca

SEQ ID NO: 21 - RNA, 34 nt, Artificial
  CRISPR guide RNA
  misc_feature (15)..(34): n is a, c, g, or u
cugagaaguggcacnnnnnnnnnnnnnnnnnnnn

SEQ ID NO: 22 - RNA, 14 nt, Artificial
  CRISPR guide RNA
cugagaaguggcac

SEQ ID NO: 23 - RNA, 9 nt, Artificial
  CRISPR guide RNA
cugagaagu

SEQ ID NO: 24 - RNA, 13 nt, Artificial
  CRISPR guide RNA
ugagaaguggcac

SEQ ID NO: 25 - RNA, 8 nt, Artificial
  CRISPR guide RNA
ugagaagu

SEQ ID NO: 26 - protein, 1368 aa, S. pyrogenes [sequence omitted]

SEQ ID NO: 27 - protein, 1300 aa, Francisella  tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 28 - protein, 1307 aa, Artificial
  Artificial CRISPR peptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 29 - protein, 1246 aa, Artificial
  Artificial CRISPR peptide
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 30 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 31 - protein, 1326 aa, Prevotella  disiens
MKVMENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 32 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 33 - protein, 872 aa, Alicyclobacillus contaminans
MGFNTAELLRKVEEEMRKTSVGFDTDNPFAHRITRRAIRGWDRIAEAWRRLPPDAPESEYIEAFKDIQRKNPRKIGSEPLFKNLAAPGVRSELLNNPQVLITFAKYNELQRQLAKAKQFAQKTLPHPVFHPVWVRYDKLGGNLHHYQIEPAVHANDTHKVKFSSLLLPQEDGSYAEVKDVTVSLAPSLQFPTGLVHPKVTTPPRTGLVTVMDEEAGKPVVCYRDRGHDALVPVAFGGAKLQFNRAHLSAGYRKGVLSAGGGGSIYFNVTLDVQVPNERDVSKTFSFSRDRDLVSLKAEELKRYMETKPLGMPGVRVMSVDLGVRYGAAISVFEVKPFAEVRKDKLHYPITGCEGFVAEHERSVILKLPGEGVRTAGKQSERKQALAAIRAEMSILRKWLRVSQVTEEDRAKAVRGLLEDERGGGWTMDPGEDSDHQPLQQFLHEARLAVGELVNLVHLSPAEWERAVIERHRRLERITASHIRVFQTMRKVWGKRRNEDAAHTGGISLAHIEHLIQQRKLFIRWSTHARTYGEVRRLPKHEGFAKRLQKHTNHVKEDRIKKLADMIVMAARGYRFLDKRARWVKTRHAPCDLILFEDLSRYRFTMDRPPTENSQLMNWSHRELLKTVKMQAALFGIGVGTVPAAFTSRFDAQTGAPGLRCKRVTKQDKEKTPFWLIQFAEITGVNVTNVEPGQLIPVDGGEWFVSPKGPRAADGLKCVHADINAAHNLQRRFWIPRLPSVKCRRYVEAEGFAAVPSSTAFMKVHGKGAFVSVDGEFYEYQKGRRVAVNRADRTSSTLDEDEGDIGEEMLVSSNGAGEFVRMFYDESGYVGYGRWMDSKVFWGKVRQIVHRAIQDQVEKRAAARGENGATSSR

SEQ ID NO: 34 - protein, 1149 aa, Desulfovibrio inopinatus
MPTRTINLKLVLGKNPENATLRRALFSTHRLVNQATKRIEEFLLLCRGEAYRTVDNEGKEAEIPRHAVQEEALAFAKAAQRHNGCISTYEDQEILDVLRQLYERLVPSVNENNEAGDAQAANAWVSPLMSAESEGGLSVYDKVLDPPPVWMKLKEEKAPGWEAASQIWIQSDEGQSLLNKPGSPPRWIRKLRSGQPWQDDFVSDQKKKQDELTKGNAPLIKQLKEMGLLPLVNPFFRHLLDPEGKGVSPWDRLAVRAAVAHFISWESWNHRTRAEYNSLKLRRDEFEAASDEFKDDFTLLRQYEAKRHSTLKSIALADDSNPYRIGVRSLRAWNRVREEWIDKGATEEQRVTILSKLQTQLRGKFGDPDLFNWLAQDRHVHLWSPRDSVTPLVRINAVDKVLRRRKPYALMTFAHPRFHPRWILYEAPGGSNLRQYALDCTENALHITLPLLVDDAHGTWIEKKIRVPLAPSGQIQDLTLEKLEKKKNRLYYRSGFQQFAGLAGGAEVLFHRPYMEHDERSEESLLERPGAVWFKLTLDVATQAPPNWLDGKGRVRTPPEVHHFKTALSNKSKHTRTLQPGLRVLSVDLGMRTFASCSVFELIEGKPETGRAFPVADERSMDSPNKLWAKHERSFKLTLPGETPSRKEEEERSIARAEIYALKRDIQRLKSLLRLGEEDNDNRRDALLEQFFKGWGEEDVVPGQAFPRSLFQGLGAAPFRSTPELWRQHCQTYYDKAEACLAKHISDWRKRTRPRPTSREMWYKTRSYHGGKSIWMLEYLDAVRKLLLSWSLRGRTYGAINRQDTARFGSLASRLLHHINSLKEDRIKTGADSIVQAARGYIPLPHGKGWEQRYEPCQLILFEDLARYRFRVDRPRRENSQLMQWNHRAIVAETTMQAELYGQIVENTAAGFSSRFHAATGAPGVRCRFLLERDFDNDLPKPYLLRELSWMLGNTKVESEEEKLRLLSEKIRPGSLVPWDGGEQFATLHPKRQTLCVIHADMNAAQNLQRRFFGRCGEAFRLVCQPHGDDVLRLASTPGARLLGALQQLENGQGAFELVRDMGSTSQMNRFVMKSLGKKKIKPLQDNNGDDELEDVLSVLPEEDDTGRITVFRDSSGIFFPCNVWIPAKQFWPAVRAMIWKVMASHSLG

SEQ ID NO: 35 - protein, 1194 aa, Desulfonatronum thiodismutans
MVLGRKDDTAELRRALWTTHEHVNLAVAEVERVLLRCRGRSYWTLDRRGDPVHVPESQVAEDALAMAREAQRRNGWPVVGEDEEILLALRYLYEQIVPSCLLDDLGKPLKGDAQKIGTNYAGPLFDSDTCRRDEGKDVACCGPFHEVAGKYLGALPEWATPISKQEFDGKDASHLRFKATGGDDAFFRVSIEKANAWYEDPANQDALKNKAYNKDDWKKEKDKGISSWAVKYIQKQLQLGQDPRTEVRRKLWLELGLLPLFIPVFDKTMVGNLWNRLAVRLALAHLLSWESWNHRAVQDQALARAKRDELAALFLGMEDGFAGLREYELRRNESIKQHAFEPVDRPYVVSGRALRSWTRVREEWLRHGDTQESRKNICNRLQDRLRGKFGDPDVFHWLAEDGQEALWKERDCVTSFSLLNDADGLLEKRKGYALMTFADARLHPRWAMYEAPGGSNLRTYQIRKTENGLWADVVLLSPRNESAAVEEKTFNVRLAPSGQLSNVSFDQIQKGSKMVGRCRYQSANQQFEGLLGGAEILFDRKRIANEQHGATDLASKPGHVWFKLTLDVRPQAPQGWLDGKGRPALPPEAKHFKTALSNKSKFADQVRPGLRVLSVDLGVRSFAACSVFELVRGGPDQGTYFPAADGRTVDDPEKLWAKHERSFKITLPGENPSRKEEIARRAAMEELRSLNGDIRRLKAILRLSVLQEDDPRTEHLRLFMEAIVDDPAKSALNAELFKGFGDDRFRSTPDLWKQHCHFFHDKAEKVVAERFSRWRTETRPKSSSWQDWRERRGYAGGKSYWAVTYLEAVRGLILRWNMRGRTYGEVNRQDKKQFGTVASALLHHINQLKEDRIKTGADMIIQAARGFVPRKNGAGWVQVHEPCRLILFEDLARYRFRTDRSRRENSRLMRWSHREIVNEVGMQGELYGLHVDTTEAGFSSRYLASSGAPGVRCRHLVEEDFHDGLPGMHLVGELDWLLPKDKDRTANEARRLLGGMVRPGMLVPWDGGELFATLNAASQLHVIHADINAAQNLQRRFWGRCGEAIRIVCNQLSVDGSTRYEMAKAPKARLLGALQQLKNGDAPFHLTSIPNSQKPENSYVMTPTNAGKKYRAGPGEKSSGEEDELALDIVEQAEELAQGRKTFFRDPSGVFFAPDRWLPSEIYWSRIRRRIWQVTLERNSSGRQERAEMDEMPY

SEQ ID NO: 36 - protein, 1132 aa, Tuberibacillus calidus
MATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNMQKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 37 - protein, 1108 aa, Bacillus thermoamylovorans
MATRSFILKIEPNEEVKKGLWKTHEVLNHGIAYYMNILKLIRQEAIYEHHEQDPKNPKKVSKAEIQAELWDFVLKMQKCNSFTHEVDKDVVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPFTDSNEPIVKEIKWMEKSRNQSVRRLDKDMFIQALERFLSWESWNLKVKEEYEKVEKEHKTLEERIKEDIQAFKSLEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKFVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKLVTTHADINAAQNLQKRFWTRTHGFYKVYCKAYQVDGQTVYIPESKDQKQKIIEEFGEGYFILKDGVYEWGNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 38 - protein, 1108 aa, Artificial
  Artificial CRISPR peptide
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 39 - protein, 658 aa, Methylobacterium nodulans
MLTKQDKQQKITYCTNMNEVFEAKLGSADLLLNWDHLRGRIRDRVDAGDIGSAFLKLALDVAHVLPDGVDDQLARAAFHFQSAKGAKSKHADSVQAGLRVLSIDLGVRSFATCSVFELKDTAPTTGVAFPLAEFRLWAVHERSFTLELPGENVGAAGQQWRAQADAELRQLRGGLNRHRQLLRAATVQKGERDAYLTDLREAWSAKELWPFEASLLSELERCSTVADPLWQDTCKRAARLYRTEFGAVVSEWRSRTRSREDRKYAGKSMWSVQHLTDVRRFLQSWSLAGRASGDIRRLDRERGGVFAKDLLDHIDALKDDRLKTGADLIVQAARGFQRNEFGYWVQKHAPCHVILFEDLSRYRMRTDRPRRENSQLMQWAHRGVPDMVGMQGEIYGIQDRRDPDSARKHARQPLAAFCLDTPAAFSSRYHASTMTPGIRCHPLRKREFEDQGFLELLKRENEGLDLNGYKPGDLVPLPGGEVFVCLNANGLSRIHADINAAQNLQRRFWTQHGDAFRLPCGKSAVQGQIRWAPLSMGKRQAGALGGFGYLEPTGHDSGSCQWRKTTEAEWRRLSGAQKDRDEAAAAEDEELQGLEEELLERSGERVVFFRDPSGVVLPTDLWFPSAAFWSIVRAKTVGRLRSHLDAQAEASYAVAAGL

SEQ ID NO: 40 - protein, 1302 aa, Artificial
  Artificial CRISPR peptide
MKKFELKQNFRNNYSGKTLRNFRQTLAQIANKKSSDSILTIKFKLDCSKTGKLPKYENLISLYDTIEDIKKGTLSYYLFTLIVSGFKFFGSASQAKAFSTKDIFKDNDFYNQFKIQSHLDLPDFVPSKIYQRLKKNVRSTNGKDNAFKASVIVAEYRKEIGKLKNKDESSEHQCEELFKKIGTALETRFSSWQDLINNCSTGCEIIDEILNDSFGTLPSIKKMVLASTTQSSDGEQDGIAIAYDPDSTFIKSDELLNPYFAVATILKSMPPEIQQDKKSAYVKANLTTPTHNALSWIFGKGLTLFQTESTEKLCAMFNVSDKRVIEQVQDAAKAVKLPAELDLNHCTLKFQDFRSSLGGHLDSWTTNYLKRLDELNDLLLNLPKNLSLPDIFMIDGKDFIEYSGCNRDEIQQMIDFVVNEQNRIKLQESLNALLGKGNNQICSDDISTVKDFSEIVNSLHSFVQQIDNSLEQSSNEANSIFSELKKKIEKNEKWDIWKNNLKKIPKLNKLSGGVPDAWKEIREIEQKFHEISENQKKHFTEVMEWIDAGNGTIDIFESRFKYDELLKKSKKNNLQSADELAFRSVLNKLGRFARQGNDLVCEKIKNWFKEQNIFDSSKDFNRYFINQKGFIFKHPSSKKDNSPYNLSANLLEKRYEVTNTVGALLEQCESDPAIVNDPFSMRSLVEFRALWFSINISGISKEQHIPTKIAQPKLDDSTYQESVSPTLKYRLEKEQITSSELNSIFTVYKSLLSGLSIRLSRNSFYLRTKFSWIGNNSLIYCPKETTWKIPAAYFKSDLWNEYKDKQILIVNEEYDVDVVKTFESVYKIVKSKDNNEKNRILPLLKQLPHDWMFKLPFGASNAEKCKVLKLEKNNKKFKPLSVSKDSLARLSGPSTYFNQIDEIMMNDESELSEMTLLADEPVRQQMSNGKIEIIPDDYVMSLAIPITRSLKKGNTESFPFKNIVSIDQGEAGFAYAVFKLSDCGNERAEPIATGLIPIPSIRRLIHSVKKYRGKKQRIQNFNQKFDSTMFTLRENVTGDICGLIVALMKKYNAFPILEKQVGNLESGSKQLMLVYKAVNSKFLAAKVDMQNDQRRSWWYQGNSWNTPILRISNPNQSNNKNIVKNINGKKYEELKIYPGYSVSAYMTSCICHVCGRNALELLKNDDSTGKVKKYQINQDGEVTIGGEVIKLYRKPDRLTPVKNLAKKGNRERTYASINERAPTVSVQKAELSADELQKIIKKNMRRAPRSLMSKDTTQSRYFCVFKNCPCHNKEQHADVNAAINIGRRFLKDCILDDNKEKD

SEQ ID NO: 41 - protein, 1255 aa, Artificial
  Artificial CRISPR peptide
MRSNYHGGRNARQWRKQISGLARRTKETVFTYKFPLETDAAEIDFDKAVQTYGIAEGVGHGSLIGLVCAFHLSGFRLFSKAGEAMAFRNRSRYPTDAFAEKLSAIMGIQLPTLSPEGLDLIFQSPPRSRDGIAPVWSENEVRNRLYTNWTGRGPANKPDEHLLEIAGEIAKQVFPKFGGWDDLASDPDKALAAADKYFQSQGDFPSIASLPAAIMLSPANSTVDFEGDYIAIDPAAETLLHQAVSRCAARLGRERPDLDQNKGPFVSSLQDALVSSQNNGLSWLFGVGFQHWKEKSPKELIDEYKVPADQHGAVTQVKSFVDAIPLNPLFDTTHYGEFRASVAGKVRSWVANYWKRLLDLKSLLATTEFTLPESISDPKAVSLFSGLLVDPQGLKKVADSLPARLVSAEEAIDRLMGVGIPTAADIAQVERVADEIGAFIGQVQQFNNQVKQKLENLQDADDEEFLKGLKIELPSGDKEPPAINRISGGAPDAAAEISELEEKLQRLLDARSEHFQTISEWAEENAVTLDPIAAMVELERLRLAERGATGDPEEYALRLLLQRIGRLANRVSPVSAGSIRELLKPVFMEEREFNLFFHNRLGSLYRSPYSTSRHQPFSIDVGKAKAIDWIAGLDQISSDIEKALSGAGEALGDQLRDWINLAGFAISQRLRGLPDTVPNALAQVRCPDDVRIPPLLAMLLEEDDIARDVCLKAFNLYVSAINGCLFGALREGFIVRTRFQRIGTDQIHYVPKDKAWEYPDRLNTAKGPINAAVSSDWIEKDGAVIKPVETVRNLSSTGFAGAGVSEYLVQAPHDWYTPLDLRDVAHLVTGLPVEKNITKLKRLTNRTAFRMVGASSFKTHLDSVLLSDKIKLGDFTIIIDQHYRQSVTYGGKVKISYEPERLQVEAAVPVVDTRDRTVPEPDTLFDHIVAIDLGERSVGFAVFDIKSCLRTGEVKPIHDNNGNPVVGTVAVPSIRRLMKAVRSHRRRRQPNQKVNQTYSTALQNYRENVIGDVCNRIDTLMERYNAFPVLEFQIKNFQAGAKQLEIVYGSVLHRYTFSGVDAHKAKRREYWYNGELWEHPYLMAKKWNEETNSMSGAPKPVSLFPGVTVNAARTSQICHQCQRNPMSHLRGLTGTIEISSDGLLELDDGTIRLFETSDYDEDKFKQSRREKRRLDANVLLSGRHRAEYIYTVAKRNLRRPPKNVMTKDTTQSRYTCLYKNCSWTGHADENAAINIGRRYLAERIDMPASKTKAAV

SEQ ID NO: 42 - protein, 1266 aa, Artificial
  Artificial CRISPR peptide
MRPRFHGGMNARDWRKHVGVLAQQHKETTRTYTFPLDTTGSAIDFDAALQAYNAVEGVGYGSLLGLACAVHLSGFRLFSTGKEAATFRNRARYPNAAFQAALRKELGTTITTLTPETLDRLFSSRPKRRNGVPLPWNQDSIRDRLYTNWVKPRPGDTPDAVLFQIATGIAQEITEDVSSWTDLAKNSDRGLKAAHRYFARVGGFPAFDNLTPPATVQPTDTTIDYDPNAPFHLVSHADQTLIHQSISLCAHRIRQEDPALDPNKSGFIKQLQNNFLSQTFYGLSWLFGAGYVHFRECTANDLAIQYGIPNNCRDGIHQIKSFADAILPNTFFEKKHYRKDSRSVGKKAKSWISNYWQRLLQLQTWVDDHTWVTLPQELTEAQFKPLFRGLLVDAVELMAIAERLPQRLADCRDSLDCLMGKGPQAATKNDVEIVEKVREEIESFVGQIEQLGNQLRHQLENENNDQVHRDNLHQLKNRLPLDLRRPQALNKISGGVPDVAKSIRGLETQLDQVLKERRSHFGRLTKWAKECGITLDPLQPLIESEKQRVAERGSAHDAKELAIRLLLQRIGRLGHRLSPTNATAIQELLRPVFAVKREFNLFFHNHMGALYRSPYSTSRHQPFQINVDVAHGTDWIGTIETLIQNLFTQIQDDALLRDLVQLEGFVFSHKLRALPGVIPSELARPNNLQQMGLPALLLVLLQADQVHRETVLRVFNLYGSAINGYLFQALRPGFIVRAGFQRLETKKLRYVPKAQSWQYPDRLHHAKSAIKNSLSAGWIKKNHQGAILPQKTLTALVKQKSLKDTGVPEYLVQAPHDWYVPIDLRGPAIPIEGLTVGTEGPELTQLGPMKDDCAFRAIGPSSFKSKIDAGLLPQDVKYGDMTLIFDQHYQQSISFANGTFSIQYQPTSLQVKAAIPVVDKRPRDTRNNSHLYDRIVAIDLGERKIGYAIFDLKQVLKSEQLEPMREDGKPLIGSISIRSIRGLMKAVQTHRNRRQPNYRIDQTYSKALMHYRESVIGDVCNAIDTLCARYGGFPVLESSVRNFEVGSAQLKTVYGSVSRRYTWSAVDAHKNQRQQYWLGGTKDKIPIWTHPYLMTREWDEKNSKWSNRSKPLKMHPGVEVHPAGTSQICHQCKRNPIGALWNVADTVVLDDQGQLDLDDGTIRLNSGYIDTTEIKRARRKKIRLPENKPLTGSHKTSHVRAVARRNLRQPPKSTRAKDTTQSRYTCLYVDCGHECHADENAAINIGRKYLQERIHIEASRQALSTR

SEQ ID NO: 43 - protein, 1269 aa, Artificial
  Artificial CRISPR peptide
MVAGLKKIKRDGVTMKSNYHGGVKARAWRKRIGGLARRQKETVFTYKFPLETEEAGIDFDKAVQTYGIAEGISQGSLIGLVCAFHLSGFRLFSKADETKAFCNQGRYPNQAFAEKLRNELSVTLPKLSPQSLDVLFQSSPKSKNGVAPEWSKNAIRNRLYTNWTGKGAGTNPDEHLLEIAEDIAAEIDSDLDGWKDLEEHPEKGLSAADRYFQAQGDFPSLTGLPPSVPLTPQNSTVAFEGDPVCLNPSDNTLLHQAVARCAGRILQEQPNLSPDKNRFINQLQDELVSSQNNGLSWLFGVGFKYWKEMSVDQLADDYKVKSTDLDALKQVKSFIDAIPLNPLFDTPHYGEFRASVAGKMRSWVKNYWKRLLDLKSQLGTANINLPEGLDEQRAENLFSGLLIDSKGLRQVTDKLPSRLKKAEDTIDRLMGDGNPTSDDIEQVETVAAEISAFIGQVEQFNNQLEQRLENPLEGDDETFLKQLKIDLPAEFKKPPAINRISGGSPDPTAEIAELEEKLDRLMSARKEHYETIAEWASANKVTLDPMEAMTTLEAQRLTERGAEGDQEEFALRLLLQRIGRLANRLSPQGATAIRDLLRPVFTEKREFNLFFHNRMGSLYRSPYSTSRHQPFTIDVAVAKNTDWMDALDGIAETIMKGLSQAGDELSLRLRDWINISGFSLSQRLRGLPDTVPGELALVRSADDVRIPPMLALQLEEDEVSREVCLKAFNLYVSAINGCLFRALREGFIVRTKFQRLERDVLSYVPKTKLWNYPQRLDTARGPIHSALAAAWINKEGSVIDPVETVTALSDTGFSDDGIPEYLVQAPHDWYTPIDLRDISKPVSGLPVKKNITGLKRQKKQTAFRMVGPSSFKSHLDSTLLSEEVKLGDFTLIFDQYYKQRVSYNGRVKITFEPDRLHVEAAVPVIDKRVRPSTEEDALFDHLLAIDLGEKRVGYAVYDIKACLRTGDIKPLEDGDGKPIVGSVAVPSIRRLMKAVRSHRQQRQPNQKVNQTYSTALMNYRENVIGDVCNRIDTLMEKYNAFPVLESSVMNFEAGSRQLEMVYGSVLHRYTYSKIDAHTAKRKEYWYTGEYWDHPYLMAHKWNERTRSYSGSLSALTLYPGVMVHPAGTSQRCHQCKRNPMVEIKQLTGQVEINADGSLELDDGTICLYEGYDYSPEEYKKAKREKRRLDPNVPLSGRHQAKHVSAVAKRNLRRPTVSMMSGDTTQARYVCLYTDCDFTGHADENAAINIGWKYLTERIALSESKDKAGV

SEQ ID NO: 44 - protein, 1340 aa, Artificial
  Artificial CRISPR peptide
MQISKVNHKHVAVGQKDRERITGFIYNDPVGDEKSLEDVVAKRANDTKVLFNVFNTKDLYDSQESDKSEKDKEIISKGAKFVAKSFNSAITILKKQNKIYSTLTSQQVIKELKDKFGGARIYDDDIEEALTETLKKSFRKENVRNSIKVLIENAAGIRSSLSKDEEELIQEYFVKQLVEEYTKTKLQKNVVKSIKNQNMVIQPDSDSQVLSLSESRREKQSSAVSSDTLVNCKEKDVLKAFLTDYAVLDEDERNSLLWKLRNLVNLYFYGSESIRDYSYTKEKSVWKEHDEQKANKTLFIDEICHITKIGKNGKEQKVLDYEENRSRCRKQNINYYRSALNYAKNNTSGIFENEDSNHFWIHLIENEVERLYNGIENGEEFKFETGYISEKVWKAVINHLSIKYIALGKAVYNYAMKELSSPGDIEPGKIDDSYINGITSFDYEIIKAEESLQRDISMNVVFATNYLACATVDTDKDFLLFSKEDIRSCTKKDGNLCKNIMQFWGGYSTWKNFCEEYLKDDKDALELLYSLKSMLYSMRNSSFHFSTENVDNGSWDTELIGKLFEEDCNRAARIEKEKFYNNNLHMFYSSSLLEKVLERLYSSHHERASQVPSFNRVFVRKNFPSSLSEQRITPKFTDSKDEQIWQSAVYYLCKEIYYNDFLQSKEAYKLFREGVKNLDKNDINNQKAADSFKQAVVYYGKAIGNATLSQVCQAIMTEYNRQNNDGLKKKSAYAEKQNSNKYKHYPLFLKQVLQSAFWEYLDENKEIYGFISAQIHKSNVEIKAEDFIANYSSQQYKKLVDKVKKTPELQKWYTLGRLINPRQANQFLGSIRNYVQFVKDIQRRAKENGNPIRNYYEVLESDSIIKILEMCTKLNGTTSNDIHDYFRDEDEYAEYISQFVNFGDVHSGAALNAFCNSESEGKKNGIYYDGINPIVNRNWVLCKLYGSPDLISKIISRVNENMIHDFHKQEDLIREYQIKGICSNKKEQQDLRTFQVLKNRVELRDIVEYSEIINELYGQLIKWCYLRERDLMYFQLGFHYLCLNNASSKEADYIKINVDDRNISGAILYQIAAMYINGLPVYYKKDDMYVALKSGKKASDELNSNEQTSKKINYFLKYGNNILGDKKDQLYLAGLELFENVAEHENIIIFRNEIDHFHYFYDRDRSMLDLYSEVFDRFFTYDMKLRKNVVNMLYNILLDHNIVSSFVFETGEKKVGRGDSEVIKPSAKIRLRANNGVSSDVFTYKVGSKDELKIATLPAKNEEFLLNVARLIYYPDMEAVSENMVREGVVKVEKSNDKKGKISRGSNTRSSNQSKYNNKSKNRMNYSMGSIFEKMDLKFD

SEQ ID NO: 45 - protein, 1437 aa, Artificial
  Artificial CRISPR peptide
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 46 - protein, 1385 aa, Clostridium aminophilum
MKFSKVDHTRSAVGIQKATDSVHGMLYTDPKKQEVNDLDKRFDQLNVKAKRLYNVFNQSKAEEDDDEKRFGKVVKKLNRELKDLLFHREVSRYNSIGNAKYNYYGIKSNPEEIVSNLGMVESLKGERDPQKVISKLLLYYLRKGLKPGTDGLRMILEASCGLRKLSGDEKELKVFLQTLDEDFEKKTFKKNLIRSIENQNMAVQPSNEGDPIIGITQGRFNSQKNEEKSAIERMMSMYADLNEDHREDVLRKLRRLNVLYFNVDTEKTEEPTLPGEVDTNPVFEVWHDHEKGKENDRQFATFAKILTEDRETRKKEKLAVKEALNDLKSAIRDHNIMAYRCSIKVTEQDKDGLFFEDQRINRFWIHHIESAVERILASINPEKLYKLRIGYLGEKVWKDLLNYLSIKYIAVGKAVFHFAMEDLGKTGQDIELGKLSNSVSGGLTSFDYEQIRADETLQRQLSVEVAFAANNLFRAVVGQTGKKIEQSKSEENEEDFLLWKAEKIAESIKKEGEGNTLKSILQFFGGASSWDLNHFCAAYGNESSALGYETKFADDLRKAIYSLRNETFHFTTLNKGSFDWNAKLIGDMFSHEAATGIAVERTRFYSNNLPMFYRESDLKRIMDHLYNTYHPRASQVPSFNSVFVRKNFRLFLSNTLNTNTSFDTEVYQKWESGVYYLFKEIYYNSFLPSGDAHHLFFEGLRRIRKEADNLPIVGKEAKKRNAVQDFGRRCDELKNLSLSAICQMIMTEYNEQNNGNRKVKSTREDKRKPDIFQHYKMLLLRTLQEAFAIYIRREEFKFIFDLPKTLYVMKPVEEFLPNWKSGMFDSLVERVKQSPDLQRWYVLCKFLNGRLLNQLSGVIRSYIQFAGDIQRRAKANHNRLYMDNTQRVEYYSNVLEVVDFCIKGTSRFSNVFSDYFRDEDAYADYLDNYLQFKDEKIAEVSSFAALKTFCNEEEVKAGIYMDGENPVMQRNIVMAKLFGPDEVLKNVVPKVTREEIEEYYQLEKQIAPYRQNGYCKSEEDQKKLLRFQRIKNRVEFQTITEFSEIINELLGQLISWSFLRERDLLYFQLGFHYLCLHNDTEKPAEYKEISREDGTVIRNAILHQVAAMYVGGLPVYTLADKKLAAFEKGEADCKLSISKDTAGAGKKIKDFFRYSKYVLIKDRMLTDQNQKYTIYLAGLELFENTDEHDNITDVRKYVDHFKYYATSDENAMSILDLYSEIHDRFFTYDMKYQKNVANMLENILLRHFVLIRPEFFTGSKKVGEGKKITCKARAQIEIAENGMRSEDFTYKLSDGKKNISTCMIAARDQKYLNTVARLLYYPHEAKKSIVDTREKKNNKKTNRGDGTFNKQKGTARKEKDNGPREFNDTGFSNTPFAGFDPFRNS

SEQ ID NO: 47 - protein, 1334 aa, Artificial
  Artificial CRISPR peptide
MKISKVDHTRMAVAKGNQHRRDEISGILYKDPTKTGSIDFDERFKKLNCSAKILYHVFNGIAEGSNKYKNIVDKVNNNLDRVLFTGKSYDRKSIIDIDTVLRNVEKINAFDRISTEEREQIIDDLLEIQLRKGLRKGKAGLREVLLIGAGVIVRTDKKQEIADFLEILDEDFNKTNQAKNIKLSIENQGLVVSPVSRGEERIFDVSGAQKGKSSKKAQEKEALSAFLLDYADLDKNVRFEYLRKIRRLINLYFYVKNDDVMSLTEIPAEVNLEKDFDIWRDHEQRKEENGDFVGCPDILLADRDVKKSNSKQVKIAERQLRESIREKNIKRYRFSIKTIEKDDGTYFFANKQISVFWIHRIENAVERILGSINDKKLYRLRLGYLGEKVWKDILNFLSIKYIAVGKAVFNFAMDDLQEKDRDIEPGKISENAVNGLTSFDYEQIKADEMLQREVAVNVAFAANNLARVTVDIPQNGEKEDILLWNKSDIKKYKKNSKKGILKSILQFFGGASTWNMKMFEIAYHDQPGDYEENYLYDIIQIIYSLRNKSFHFKTYDHGDKNWNRELIGKMIEHDAERVISVEREKFHSNNLPMFYKDADLKKILDLLYSDYAGRASQVPAFNTVLVRKNFPEFLRKDMGYKVHFNNPEVENQWHSAVYYLYKEIYYNLFLRDKEVKNLFYTSLKNIRSEVSDKKQKLASDDFASRCEEIEDRSLPEICQIIMTEYNAQNFGNRKVKSQRVIEKNKDIFRHYKMLLIKTLAGAFSLYLKQERFAFIGKATPIPYETTDVKNFLPEWKSGMYASFVEEIKNNLDLQEWYIVGRFLNGRMLNQLAGSLRSYIQYAEDIERRAAENRNKLFSKPDEKIEACKKAVRVLDLCIKISTRISAEFTDYFDSEDDYADYLEKYLKYQDDAIKELSGSSYAALDHFCNKDDLKFDIYVNAGQKPILQRNIVMAKLFGPDNILSEVMEKVTESAIREYYDYLKKVSGYRVRGKCSTEKEQEDLLKFQRLKNAVEFRDVTEYAEVINELLGQLISWSYLRERDLLYFQLGFHYMCLKNKSFKPAEYVDIRRNNGTIIHNAILYQIVSMYINGLDFYSCDKEGKTLKPIETGKGVGSKIGQFIKYSQYLYNDPSYKLEIYNAGLEVFENIDEHDNITDLRKYVDHFKYYAYGNKMSLLDLYSEFFDRFFTYDMKYQKNVVNVLENILLRHFVIFYPKFGSGKKDVGIRDCKKERAQIEISEQSLTSEDFMFKLDDKAGEEAKKFPARDERYLQTIAKLLYYPNEIEDMNRFMKKGETINKKVQFNRKKKITRKQKNNSSNEVLSSTMGYLFKNIKL

SEQ ID NO: 48 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 49 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 50 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 51 - protein, 1051 aa, Listeria newyorkensis
MKITKMRVDGRTIVMERTSKEGQLGYEGIDGNKTTEIIFDKKKESFYKSILNKTVRKPDEKEKNRRKQAINKAINKEITELMLAVLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKLVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGNFKKNLPNSTTRSDVVKKMIPFFSDELQNVNFDEAIWAIRGSIQQIRNEVYHCKKHSWKSILKIKGFEFEPNNMKYADSDMQKLMDKDIAKIPEFIEEKLKSSGVVRFYRHDELQSIWEMKQGFSLLTTNAPFVPSFKRVYAKGHDYQTSKNRYYNLDLTTFDILEYGEEDFRARYFLTKLVYYQQFMPWFTADNNAFRDAANFVLRLNKNRQQDAKAFINIREVEEGEMPRDYMGYVQGQIAIHEDSIEDTPNHFEKFISQVFIKGFDRHMRSANLKFIKNPRNQGLEQSEIEEMSFDIKVEPSFLKNKDDYIAFWIFCKMLDARHLSELRNEMIKYDGHLTGEQEIIGLALLGVDSRENDWKQFFSSEREYEKIMKGYVVEELYQREPYRQSDGKTPILFRGVEQARKYGTETVIQRLFDANPEFKVSKCNLAEWERQKETIEETIKRRKELHNEWAKNPKKPQNNAFFKEYKECCDAIDAYNWHKNKTTLAYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGGKKIDGGYIETNQVSEEYCGIVKRLLEM

SEQ ID NO: 52 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 53 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 54 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 55 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 56 - protein, 8 aa, Artificial
  Transferring receptor binding protein
  misc_feature (8)..(8): Xaa can be any naturally occurring amino acid
  misc_feature (11)..(11): Xaa is propargylglycine
HAIYPRHX

SEQ ID NO: 57 - protein, 13 aa, Artificial
  Transferring receptor binding protein
  misc_feature (13)..(13): Xaa is propargylglycine
THRPPMWSPVWPX

(19) United States
(12) Patent Application Publication
(10) Pub. No.: US 2023/0285291 A1
(43) Pub. Date: Sep. 14, 2023

(54) MANUFACTURING OF SYNTHETIC EXOSOMES FOR CNS AND NON-CNS DELIVERY OF THERAPEUTICS

(71) Applicant: The Regents of the University of California, Oakland, CA (US)

(72) Inventors: Jesus Campagna, Playa Del Rey, CA (US); Varghese John, San Francisco, CA (US)

(73) Assignee: The Regents of the University of California, Oakland, CA (US)

(21) Appl. No.: 17/917,233

(22) PCT Filed: Apr. 6, 2021
(86) PCT No.: PCT/US2021/026049

§ 371 Date: Oct. 5, 2022

Related U.S. Application Data
(60) Provisional application No. 63/006,593, filed on Apr. 7, 2020.

(51) Int. Cl. A61K 9/127; A61K 38/46; A61K 38/47; B01J 19/00

(52) U.S. Cl. CPC A61K 9/127; A61K 38/465; A61K 38/47; A61K 9/1277; B01J 19/0093; B01J 2219/00889; B01J 2219/00894

(57) ABSTRACT

This invention provides improved synthetic exosomes for delivery a one or more therapeutic agents to the central nervous system. In certain embodiments the synthetic exosome comprises a liposome formed from a lipid bilayer, where said lipid bilayer comprises: one or more phospholipids selected from the group consisting of phosphate lipids, phosphoglycerol lipids, phosphocholine lipids, and phosphoethanolamine lipids where the lipid carbon chain ranges from 3 to 24 carbon atoms; cholesterol, a cholesterol derivative (e.g., cholesterol hemicsuccinate), or a phytosterol; and a non-ionic surfactant; wherein the lipid bilayer does not contain an alcohol; and the liposome ranges in size from about 50 nm up to about 200 nm in diameter. Typically, the synthetic exosome is capable of crossing the blood brain barrier without substantially leaking said therapeutic moiety.

### CROSS-REFERENCE TO RELATED APPLICATIONS

This application is a U.S. 371 National Phase of PCT/US2021/026049, filed Apr. 6, 2021, which claims priority to and benefit of U.S. Ser. No. 63/006,593, filed on Apr. 7, 2020, each of which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS A TEXT FILE

This application contains references to nucleic acid sequences that have been submitted concurrently herewith as the sequence listing text file “UCLA-P221US_ST25.txt”, file size 320,230 bytes, created on Apr. 5, 2023, which is incorporated by reference in its entirety pursuant to 37 C.F.R. § 1.52(e)(5).

### BACKGROUND

Exosomes are nano-sized vesicles (e.g., less than 200 nm) that serve as mediators for intercellular communication through the delivery of various endogenous cargos, including proteins, lipids, nucleic acids or other cellular components, to neighboring or distant cells. Exosome cargos may vary in response to different physiological or pathological conditions.

Due to the critical role of exosomes in intercellular communications in delivering cargo to recipient cells, exosomes have been investigated as a vector for the delivery of endogenous or exogenous cargo for therapeutic purposes. But the number of exosomes produced by cells is limited, which hampers their application. Additionally, the production of exosomes from cells is a tedious, low yield process that is often not well-controlled. Moreover, the essential components of active exosomes are not well established. Finally, fundamental mechanisms of exosomal delivery are currently unclear. Such issues have challenged the development of exosomes for the delivery of therapeutic agents.

### SUMMARY

Various embodiments provided herein may include, but need not be limited to, one or more of the following:

Embodiment 1: A synthetic exosome capable of delivering a therapeutic moiety across the blood brain barrier into the central nervous system (CNS), said synthetic exosome comprising:
a liposome formed from a lipid bilayer, where said lipid bilayer comprises:
   one or more phospholipids selected from the group consisting of phosphate lipids, phosphoglycerol lipids, phosphocholine lipids, and phosphoethanolamine lipids where the lipid carbon chain ranges from 3 to 24 carbon atoms;
   cholesterol, a cholesterol derivative, or a phytosterol; and
   a non-ionic surfactant; wherein said lipid bilayer does not contain an alcohol; and said liposome ranges in size from up to about 500 nm in diameter.

Embodiment 2: The synthetic exosome of embodiment 1, wherein said exosome is less than about 200 nm in diameter or less than about 150 nm in diameter.

Embodiment 3: The synthetic exosome of embodiment 1, wherein said exosome is about 50 nm up to about 200 nm in diameter or about 50 nm up to about 150 nm in diameter.

Embodiment 4: The synthetic exosome according to any one of embodiment 1-3, wherein said synthetic exosome is capable of crossing the blood brain barrier without substantially leaking said therapeutic moiety.

Embodiment 5: The synthetic exosome according to any one of embodiments 1-4, wherein said lipid bilayer consists of said one or more phospholipids, said cholesterol or cholesterol derivative or a phytosterol; and said non-ionic surfactant.

Embodiment 6: The synthetic exosome according to any one of embodiments 1-5, wherein said exosome is capable of crossing the blood/brain barrier (BBB) and delivering a therapeutic moiety contained therein to the central nervous system without substantial loss of said therapeutic moiety.

Embodiment 7: The synthetic exosome of embodiment 6, wherein said exosome is capable of crossing the blood/brain barrier (BBB) and delivering a therapeutic moiety contained therein to the central nervous system without losing more than about 40%, or without losing more than 30%, or without losing more than 20%, or without losing more than 10%, or without losing more than 5%, or without losing more than 3%, or without losing more than 1% of a therapeutic moiety contained therein.

Embodiment 8: The synthetic exosome according to any one of embodiments 1-7, wherein said lipid bilayer does not contain an alcohol.

Embodiment 9: The synthetic exosome of embodiment 8, wherein said lipid bilayer does not contain ethanol.

Embodiment 10: The synthetic exosome according to any one of embodiments 1-9, wherein said bilayer does not contain glutathione-maleimide-PEG2000-distearoyl phosphatidyl ethanolamine.

Embodiment 11: The synthetic exosome according to any one of embodiments 1-10, wherein said exosome is not a transferosome.

Embodiment 12: The synthetic exosome according to any one of embodiments 1-11, wherein said exosome is not an ethosome.

Embodiment 13: The synthetic exosome according to any one of embodiments 1-12, wherein the molar ratio of total phospholipid to cholesterol, cholesterol, or phytosterol ranges from about 6-10 moles of total phospholipid to about 1-3 moles of cholesterol.

Embodiment 14: The synthetic exosome according to any one of embodiments 1-13, wherein the amount of surfactant ranges from about 1%, or from about 3%, or from about 5%, or from about 8% up to about 18%, or up to about 15%, or up to about 13%, or up to about 10% (wt/wt).

Embodiment 15: The synthetic exosome according to any one of embodiments 1-14, wherein said surfactant comprise one or more surfactants selected from the group consisting of Span 80, Tween 20, BRIJ® 76 (stearyl poly(10)oxy ethylene ether), BRIJ® 78 (stearyl poly(20)oxyethylene ether), BRIJ® 96 (oleyl poly(10)oxy ethylene ether), and BRIJ® 721 (stearyl poly (21) oxyethylene ether).

Embodiment 16: The synthetic exosome of embodiment 15, wherein said surfactant comprises or consists of Span 80.

Embodiment 17: The synthetic exosome of embodiment 16, wherein the lipid bilayer comprises about 10% to about 20%, or about 15% Span 80 by weight.

Embodiment 18: The synthetic exosome according to any one of embodiments 1-17, wherein said cholesterol, cholesterol derivative, or phytosterol comprises or consists of cholesterol.

Embodiment 19: The synthetic exosome according to any one of embodiments 1-17, wherein said cholesterol, cholesterol derivative, or phytosterol comprises or consists of a cholesterol derivative selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside.

Embodiment 20: The synthetic exosome according to any one of embodiments 1-17, wherein said cholesterol, cholesterol derivative comprises or consists cholesterol hemisuccinate.

Embodiment 21: The synthetic exosome according to any one of embodiments 1-17, wherein said cholesterol, cholesterol derivative, or phytosterol comprises or consists of a phytosterol.

Embodiment 22: The synthetic exosome of embodiment 21, wherein said phytosterol comprises a 9,10-secosteroid.

Embodiment 23: The synthetic exosome of embodiment 22, wherein said 9,10 secosteroid comprises a compound selected from the group consisting of vitamin D3, vitamin D2, calcipotriol.

Embodiment 24: The synthetic exosome of embodiment 21, wherein said phytosterol comprises a C-24 alkyl steroid.

Embodiment 25: The synthetic exosome of embodiment 24, wherein said C-24 alkyl steroid comprises a compound selected from the group consisting of stigmasterol, and β-sitosterol.

Embodiment 26: The synthetic exosome of embodiment 21, wherein said phytosterol comprises a pentacyclic steroid.

Embodiment 27: The synthetic exosome of embodiment 26, wherein said pentacyclic steroid comprises a compound selected from the group consisting of betulin, lupeol, ursolic acid, and oleanolic acid.

Embodiment 28: The synthetic exosome according to any one of embodiments 1-27, wherein said cholesterol, cholesterol derivative, or phytosterol is pegylated.

Embodiment 29: The synthetic exosome according to any one of embodiments 1-28, wherein said one or more phospholipids comprises one or more phospholipids selected from the group consisting of dihexanoyl-sn-glycero-3-phosphate (DHPA), didecanoyl-sn-glycero-3-phosphate (DDPA), distearoyl-sn-glycero-3-phosphate (DTPA), and dihexadecyl phosphate (DHP).

Embodiment 30: The synthetic exosome according to any one of embodiments 1-29, wherein said one or more phospholipids comprises one or more phosphoglycerol lipids selected from the group consisting of dihexanoyl-sn-glycero-3-phospho-(1′-rac-glycerol) (DHPG), dilauroyl-sn-glycero-3-phospho-(1′-rac-glycerol) (DLPG), and distearoyl-sn-glycero-3-phospho-(1′-rac-glycerol) (DTPG).

Embodiment 31: The synthetic exosome according to any one of embodiments 1-30, wherein said one or more phospholipids comprises one or more phosphocholine lipids selected from the group consisting of dipropionyl-sn-glycero-3-phosphocholine (PC), diheptanoyl-sn-glycero-3-phosphocholine (DHPC), dimyristoyl-sn-glycero-3-phosphocholine (DMPC), and dilignoceroyl-sn-glycero-3-phosphocholine (DGPC).

Embodiment 32: The synthetic exosome according to any one of embodiments 1-30, wherein said one or more phospholipids comprises one or more phosphoethanolamine lipids selected from the group consisting of sihexanoyl-sn-glycero-3-phosphoethanolamine (DHPE), and distearoyl-sn-glycero-3-phosphoethanolamine (DTPE).

Embodiment 33: The synthetic exosome according to any one of embodiments 1-30, wherein said one or more phospholipids comprises one or more phosphoethanolamine-PEG lipids selected from the group consisting of dipalmitoyl-sn-glycero-3-phospho(ethylene glycol) (DPPEG1), dimyristoyl-sn-glycero-3-phosphoethanolamine-N-[methoxy(polyethylene glycol)-350 (DMPEG350), distearoyl-sn-glycero-3-phosphoethanolamine-N-[methoxy(polyethylene glycol)-350] (DTPEG350), dimyristoyl-sn-glycero-3-phosphoethanolamine-N-[methoxy(polyethylene glycol)-550] (DMPEG550), and dimyristoyl-sn-glycero-3-phosphoethanolamine-N-[methoxy(polyethylene glycol)-1000] (DMPEG1000).

Embodiment 34: The synthetic exosome according to any one of embodiments 1-30, wherein said one or more phospholipids comprises one or more phospholipids selected from the group consisting of dioleoyl-sn-glycero-3-phosphocholine (N-aminoethyl) (PC-NH₂), diphytanoyl-sn-glycero-3-phosphoethanolamine, dioleoyl-3-trimethylammonium-propane (DOTAP), distearoyl-3-trimethylammonium-propane (DSTAP), dimyristoyl-3-trimethylammonium-propane (DMTAP), and di-O-octadecyl-sn-glycero-3-phosphocholin (DOPC).

Embodiment 35: The synthetic exosome according to any one of embodiments 1-34, wherein said one or more phospholipids is functionalized with a targeting moiety selected from the group consisting of transferrin, an amino acid, a blood brain barrier targeting antibody, insulin, folic acid, and low density lipoprotein receptor related protein 1.

Embodiment 36: The synthetic exosome according to any one of embodiments 1-35, wherein said lipid bilayer comprises or consists of:
said surfactant; and
3:2:1 molar ratio (DHPA:DHP:CH), 1:5:1 molar ratio (DHPG:DHPA:CH), 2:5:1:2 molar ratio (DHPG:DHPA:PC-NH2:CH), or 2:4:1:1:2 molar ratio (DHPG:DHPA:PC-NH2:DMPEG350:CH) to provide synthetic exosomes having a zeta potential of about −20 mV or lower.

Embodiment 37: The synthetic exosome of embodiment 36, wherein said lipid bilayer comprises or consists of said surfactant and 3:2:1 molar ratio (DHPA:DHP:CH).

Embodiment 38: The synthetic exosome of embodiment 36, wherein said lipid bilayer comprises or consists of said surfactant and 1:5:1 molar ratio (DHPG:DHPA:CH).

Embodiment 39: The synthetic exosome of embodiment 36, wherein said lipid bilayer comprises or consists of said surfactant and 2:5:1:2 molar ratio (DHPG:DHPA:PC-NH2:CH).

Embodiment 40: The synthetic exosome of embodiment 36, wherein said lipid bilayer comprises or consists of said surfactant and 2:4:1:1:2 molar ratio (DHPG:DHPA:PC-NH2:DMPEG350:CH).

Embodiment 41: The synthetic exosome according to any one of embodiments 1-35, wherein said lipid bilayer comprises or consists of:
said surfactant; and
2:2:1 molar ratio (DHPG:DHPC:CH), 4:4:1:2 molar ratio (DHPG:DHPA:PC-NH2:CH), 2:2:1 molar ratio (DHPG:DHPA:CH), 4:4:1:1:2 molar ratio (DHPG:DHPA:PC-NH2:DMPEG550:CH), 2:2:1 molar ratio (DHPG:DTPE:CH), 2:2:1 molar ratio (DHPG:DMTAP:CH), 4:4:1:2 molar ratio (DHPG:DMTAP:PC-NH2:CH), or 4:4:1:1:2 molar ratio (DHPG:DMTAP:PC-NH2:DMPEG550:CH) to provide synthetic exosomes having a zeta potential ranging from about −20 mV to about 20 mV.

Embodiment 42: The synthetic exosome of embodiment 41, wherein said lipid bilayer comprises or consists of said surfactant and 2:2:1 molar ratio (DHPG:DHPC:CH).

Embodiment 43: The synthetic exosome of embodiment 41, wherein said lipid bilayer comprises or consists of said surfactant and 4:4:1:2 molar ratio (DHPG:DHPA:PC-NH2:CH).

Embodiment 44: The synthetic exosome of embodiment 41, wherein said lipid bilayer comprises or consists of said surfactant and 2:2:1 molar ratio (DHPG:DHPA:CH).

Embodiment 45: The synthetic exosome of embodiment 41, wherein said lipid bilayer comprises or consists of said surfactant and 4:4:1:1:2 molar ratio (DHPG:DHPA:PC-NH2:DMPEG550:CH).

Embodiment 46: The synthetic exosome of embodiment 41, wherein said lipid bilayer comprises or consists of said surfactant and 2:2:1 molar ratio (DHPG:DTPE:CH).

Embodiment 47: The synthetic exosome of embodiment 41, wherein said lipid bilayer comprises or consists of said surfactant and 2:2:1 molar ratio (DHPG:DMTAP:CH).

Embodiment 48: The synthetic exosome of embodiment 41, wherein said lipid bilayer comprises or consists of said surfactant and 4:4:1:2 molar ratio (DHPG:DMTAP:PC-NH2:CH).

Embodiment 49: The synthetic exosome of embodiment 41, wherein said lipid bilayer comprises or consists of said surfactant and 4:4:1:1:2 molar ratio (DHPG:DMTAP:PC-NH2:DMPEG550:CH).

Embodiment 50: The synthetic exosome according to any one of embodiments 1-35, wherein said lipid bilayer comprises or consists of:
said surfactant; and
2:4:1 molar ratio (DHPC:DTPE:CH), 2:4:1 molar ratio (DHPC:DOTAP:CH), 2:4:1:2 molar ratio (DHPC:DMTAP:PC-NH2:CH), or 2:4:1:1:2 molar ratio (DHPC:DMTAP:PC-NH2:DMPEG350:CH) to provide synthetic exosomes having a zeta potential of about 20 mV or greater.

Embodiment 51: The synthetic exosome of embodiment 50, wherein said lipid bilayer comprises or consists of said surfactant and 2:4:1 molar ratio (DHPC:DTPE:CH).

Embodiment 52: The synthetic exosome of embodiment 50, wherein said lipid bilayer comprises or consists of said surfactant and 2:4:1 molar ratio (DHPC:DOTAP:CH).

Embodiment 53: The synthetic exosome of embodiment 50, wherein said lipid bilayer comprises or consists of said surfactant and 2:4:1:2 molar ratio (DHPC:DMTAP:PC-NH2:CH).

Embodiment 54: The synthetic exosome of embodiment 50, wherein said lipid bilayer comprises or consists of said surfactant and 2:4:1:1:2 molar ratio (DHPC:DMTAP:PC-NH2:DMPEG350:CH).

Embodiment 55: The synthetic exosome according to any one of embodiments 1-35, wherein said lipid bilayer comprises or consists of: said surfactant; and 4:2:1 molar ratio (DTPA:DHP:CH), 1:5:1 molar ratio (DTPG:DTPA:CH), 1:5:1:2 molar ratio (DTPG:DTPA:PC-NH2:CH), or 1:4:1:1:2 molar ratio (DTPG:DTPA:PC-NH2:DMPEG350:CH) to provide synthetic exosomes having a zeta potential of about −20 mV or lower.

Embodiment 56: The synthetic exosome of embodiment 55, wherein said lipid bilayer comprises or consists of said surfactant and 4:2:1 molar ratio (DTPA:DHP:CH).

Embodiment 57: The synthetic exosome of embodiment 55, wherein said lipid bilayer comprises or consists of said surfactant and 1:5:1 molar ratio (DTPG:DTPA:CH).

Embodiment 58: The synthetic exosome of embodiment 55, wherein said lipid bilayer comprises or consists of said surfactant and 1:5:1:2 molar ratio (DTPG:DTPA:PC-NH2:CH).

Embodiment 59: The synthetic exosome of embodiment 55, wherein said lipid bilayer comprises or consists of said surfactant and 1:4:1:1:2 molar ratio (DTPG:DTPA:PC-NH2:DMPEG350:CH).

Embodiment 60: The synthetic exosome according to any one of embodiments 1-35, wherein said lipid bilayer comprises or consists of:
said surfactant; and
2:2:1 molar ratio (DTPG:DGPC:CH), 4:4:1:2 molar ratio (DTPG:DDPA:PC-NH2:CH), 2:2:1 molar ratio (DTPG:DDPA:CH), 4:4:1:1:2 molar ratio (DTPG:DDPA:PC-NH2:DMPEG550:CH), 2:2:1 molar ratio (DTPG:DTPE:CH), 2:2:1 molar ratio (DTPG:DMTAP:CH), 4:4:1:2 molar ratio (DTPG:DMTAP:PC-NH2:CH), or 4:4:1:1:2 molar ratio (DTPG:DMTAP:PC-NH2:DMPEG550:CH) to provide synthetic exosomes having a zeta potential ranging from about −20 mV to about 20 mV.

Embodiment 61: The synthetic exosome of embodiment 60, wherein said lipid bilayer comprises or consists of said surfactant and 2:2:1 molar ratio (DTPG:DGPC:CH).

Embodiment 62: The synthetic exosome of embodiment 60, wherein said lipid bilayer comprises or consists of said surfactant and 4:4:1:2 molar ratio (DTPG:DDPA:PC-NH2:CH).

Embodiment 63: The synthetic exosome of embodiment 60, wherein said lipid bilayer comprises or consists of said surfactant and 2:2:1 molar ratio (DTPG:DDPA:CH).

Embodiment 64: The synthetic exosome of embodiment 60, wherein said lipid bilayer comprises or consists of said surfactant and 4:4:1:1:2 molar ratio (DTPG:DDPA:PC-NH2:DMPEG550:CH).

Embodiment 65: The synthetic exosome of embodiment 60, wherein said lipid bilayer comprises or consists of said surfactant and 2:2:1 molar ratio (DTPG:DTPE:CH).

Embodiment 66: The synthetic exosome of embodiment 60, wherein said lipid bilayer comprises or consists of said surfactant and 2:2:1 molar ratio (DTPG:DMTAP:CH).

Embodiment 67: The synthetic exosome of embodiment 60, wherein said lipid bilayer comprises or consists of said surfactant and 4:4:1:2 molar ratio (DTPG:DMTAP:PC-NH2:CH).

Embodiment 68: The synthetic exosome of embodiment 60, wherein said lipid bilayer comprises or consists of said surfactant and 4:4:1:1:2 molar ratio (DTPG:DMTAP:PC-NH2:DMPEG550:CH).

Embodiment 69: The synthetic exosome according to any one of embodiments 1-35, wherein said lipid bilayer comprises or consists of:
said surfactant; and
2:4:1 molar ratio (DMPC:DTPE:CH), 2:4:1 molar ratio (DMPC:DOTAP:CH), 2:4:1:2 molar ratio (DMPC:DMTAP:PC-NH2:CH), or 2:4:1:1:2 molar ratio (DMPC:DMTAP:PC-NH2:DMPEG350:CH) to provide synthetic exosomes having a zeta potential of about 20 mV or greater.

Embodiment 70: The synthetic exosome of embodiment 69, wherein said lipid bilayer comprises or consists of said surfactant and 2:4:1 molar ratio (DMPC:DTPE:CH).

Embodiment 71: The synthetic exosome of embodiment 69, wherein said lipid bilayer comprises or consists of said surfactant and 2:4:1 molar ratio (DMPC:DOTAP:CH).

Embodiment 72: The synthetic exosome of embodiment 69, wherein said lipid bilayer comprises or consists of said surfactant and 2:4:1:2 molar ratio (DMPC:DMTAP:PC-NH2:CH).

Embodiment 73: The synthetic exosome of embodiment 69, wherein said lipid bilayer comprises or consists of said surfactant and 2:4:1:1:2 molar ratio (DMPC:DMTAP:PC-NH2:DMPEG350:CH).

Embodiment 74: The synthetic exosome according to any one of embodiments 36-73, wherein CH is a cholesterol derivative.

Embodiment 75: The synthetic exosome of embodiment 74, wherein said cholesterol derivative is selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside.

Embodiment 76: The synthetic exosome of embodiment 75, wherein CH is cholesterol hemisuccinate.

Embodiment 77: The synthetic exosome according to any one of embodiments 36-73, wherein CH is a phytosterol.

Embodiment 78: The synthetic exosome of embodiment 77, wherein CH is a C-24 alkyl steroid.

Embodiment 79: The synthetic exosome according to any one of embodiments 1-78, wherein said exosomes range in size from about 50 nm up, or from about 60 nm, or from about 70 nm, or from about 80 nm, or from about 90 nm, up to about 200 nm, or up to about 150 nm, or up to about 100 nm nm average diameter.

Embodiment 80: The synthetic exosome of embodiment 79, wherein the synthetic exosome is about 50 nm average diameter, or about 100 nm average diameter, or about 150 nm average diameter.

Embodiment 81: The synthetic exosome according to any one of embodiments 1-80, wherein a targeting moiety comprising or consisting of transferrin is attached to said exosome.

Embodiment 82: The synthetic exosome according to any one of embodiments 1-80, wherein a targeting moiety comprising or consisting of folic acid is attached to said exosome.

Embodiment 83: The synthetic exosome according to any one of embodiments 1-80, wherein a targeting moiety comprising or consisting of an amino acid is attached to said exosome.

Embodiment 84: The synthetic exosome of embodiment 83, wherein said exosome is attached to an amino acid that is transported by an amino acid transporter.

Embodiment 85: The synthetic exosome according to any one of embodiments 1-80, wherein a targeting moiety comprising or consisting of insulin is attached to said exosome.

Embodiment 86: The synthetic exosome according to any one of embodiments 1-80, wherein a targeting moiety comprising or consisting of low density lipoprotein receptor related protein 1 is attached to said exosome.

Embodiment 87: The synthetic exosome according to any one of embodiments 1-80, wherein a targeting moiety comprising or consisting of a blood brain barrier targeting antibody is attached to said exosome.

Embodiment 88: The synthetic exosome according to any one of embodiments 1-80, wherein said exosome is attached to an antibody or a ligand that binds to a moiety selected from the group consisting of a transferrin receptor, an insulin receptor, an insulin growth factor receptor (IGF1R), a low-density lipoprotein (LDL) receptor, basigin, Glut1, CD98hc, and TMEM30A(cdc50A).

Embodiment 89: The synthetic exosome of embodiment 88, wherein said exosome is attached to a transferrin receptor peptide comprising a sequence selected from the group consisting of NH₂-His-Ala-Ile-Tyr-Pro-Arg-His-Pra-CONH₂ (SEQ ID NO:56), and NH₂-Thr-His-Arg-Pro-Pro-Met-Trp-Ser-Pro-Val-Trp-Pro-Pra-CONH₂ (SEQ ID NO:57).

Embodiment 90: The synthetic exosome of embodiment 89, wherein said transferrin receptor peptide is attached to said synthetic exosome using click chemistry.

Embodiment 91: The synthetic exosome according to any one of embodiments 1-80, wherein said exosome is attached to an antibody or a ligand that binds to a cell surface marker.

Embodiment 92: The synthetic exosome of embodiment 91, wherein said cell surface marker is a marker of neural or glial cells.

Embodiment 93: The synthetic exosome of embodiment 91, wherein said cell surface marker is selected from the group consisting of CD63, CD81, CD9, and CD171, and is incorporated in the lipid bilayer of said exosome.

Embodiment 94: The synthetic exosome of embodiment 93, wherein said cell surface marker is CD63.

Embodiment 95: The synthetic exosome according to any one of embodiments 93-94, wherein said cell surface marker is CD81.

Embodiment 96: The synthetic exosome according to any one of embodiments 93-95, wherein said cell surface marker is CD9.

Embodiment 97: The synthetic exosome according to any one of embodiments 93-96, wherein said cell surface marker is CD171.

Embodiment 98: The synthetic exosome according to any one of embodiments 1-97, wherein said exosome contains one or more therapeutic moieties.

Embodiment 99: The synthetic exosome of embodiment 98, wherein said therapeutic moiety is selected from the group consisting of a protein, an antibody, an enzyme, a DNA encoding an inhibitory RNA, an inhibitory RNA or a micoRNA (miRNA), a nucleic acid encoding a CRISPR endonuclease and a guide RNA, a CRISPR endonuclease and a guide RNA, and a small organic molecule.

Embodiment 100: The synthetic exosome according to any one of embodiments 98-99, wherein said synthetic exosome is effective to deliver said therapeutic moiety to the brain of a mammal after systemic administration.

Embodiment 101: The synthetic exosome according to any one of embodiments 98-100, wherein said therapeutic moiety comprises an sAPPα protein.

Embodiment 102: The synthetic exosome of embodiment 101, wherein said sAPPα is a recombinantly expressed sAPPα.

Embodiment 103: The synthetic exosome of embodiment 101, wherein said sAPPα is an isolated and purified sAPPα.

Embodiment 104: The synthetic exosome according to any one of embodiments 101-103, wherein said sAPPα is a human sAPPα.

Embodiment 105: The synthetic exosome according to any one of embodiments 98-100, wherein said therapeutic moiety comprises IDUA (e.g., for MPS1) or acid sphingomyelinase (ASM) for Niemann Pick disease.

Embodiment 106: The synthetic exosome according to any one of embodiments 98-100, wherein said therapeutic moiety comprises an antibody.

Embodiment 107: The synthetic exosome of embodiment 106, wherein said antibody comprises an antibody selected from the group consisting of full-length immunoglobulins, Fab, Fab′, Fab′-SH, F(ab)₂, Fv, Fv′, Fd, Fd′, scFv, hsFv fragments, single-chain antibodies, and cameloid antibodies.

Embodiment 108: The synthetic exosome of embodiment 107, wherein said antibody comprises a full length (intact) human immunoglobulin.

Embodiment 109: The synthetic exosome of embodiment 108, wherein said antibody comprise an IgG, or an IgA.

Embodiment 110: The synthetic exosome according to any one of embodiments 106-109, wherein said antibody comprises an antibody for the treatment of a neurodegenerative condition or for the treatment of a cancer.

Embodiment 111: The synthetic exosome of embodiment 110, wherein said antibody comprise an antibody for the treatment of a neurodegenerative condition selected from the group consisting of Alzheimer's disease, amyotrophic lateral sclerosis (ALS), Huntington's disease, and Parkinson's disease.

Embodiment 112: The synthetic exosome of embodiment 111, wherein said antibody comprises an antibody for the treatment of Alzheimer's disease.

Embodiment 113: The synthetic exosome of embodiment 112, wherein said antibody binds to a target selected from the group consisting of Aβ, mutant Aβ, tau, mutant tau, apoE, and α-synuclein.

Embodiment 114: The synthetic exosome of embodiment 113, wherein said antibody comprises an antibody selected from the group consisting of AAB-003, Bapineuzumab, Ponezumab, RG7345, Solanezumab, GSK933776, JNJ-63733657, BIIB076, LY2599666, MEDI1314, SAR228810, BAN2401, BIIB092, C2B8E12, LY3002813, LY3303560, RO 7105705, Aducanumab, Crenezumab, PRX002 (prasinezumab), and Gantenerumab, or combinations thereof.

Embodiment 115: The synthetic exosome of embodiment 113, wherein said antibody comprise an anti-pyroglutamate-3 Aβ antibody.

Embodiment 116: The synthetic exosome of embodiment 115, wherein said antibody comprises the 9D5 antibody.

Embodiment 117: The synthetic exosome of embodiment 115, wherein said antibody comprises an anti-tau antibody.

Embodiment 118: The synthetic exosome of embodiment 117, wherein said anti-tau antibody is selected from the group consisting of BIIB092, ABBV-8E12, R07105705, LY3303560, RG7345, R06926496, JNJ63733657, and UCB0107.

Embodiment 119: The synthetic exosome of embodiment 113, wherein said antibody comprise an anti-ApoE antibody.

Embodiment 120: The synthetic exosome of embodiment 111, wherein said antibody comprises an antibody for the treatment of amyotrophic lateral sclerosis (ALS).

Embodiment 121: The synthetic exosome of embodiment 120, wherein said antibody comprises an antibody that binds to a misfolded SOD1 species.

Embodiment 122: The synthetic exosome of embodiment 111, wherein said antibody comprises an antibody for the treatment of Huntington's disease.

Embodiment 123: The synthetic exosome of embodiment 122, wherein said antibody comprises an anti-SEMA4D antibody (e.g., VX15).

Embodiment 124: The synthetic exosome of embodiment 111, wherein said antibody comprises an antibody for the treatment of Parkinson's disease.

Embodiment 125: The synthetic exosome of embodiment 122, wherein said antibody comprises an anti-α-synuclein antibody (e.g., prasinezumab).

Embodiment 126: The synthetic exosome of embodiment 106, wherein said antibody comprise an antibody for the treatment of a cancer.

Embodiment 127: The synthetic exosome of embodiment 126, wherein said antibody comprises a checkpoint PD-1 blocker.

Embodiment 128: The synthetic exosome of embodiment 127, wherein said antibody comprises Keytuda for treatment of Gliomas and Brain cancer.

Embodiment 129: The synthetic exosome of embodiments 98-100, wherein said synthetic exosome contains an enzyme for enzyme replacement therapy (ERT).

Embodiment 130: The synthetic exosome of embodiments 98-100, wherein said synthetic exosome contains components of a CRISPR/Cas system for the treatment of Alzheimer's disease, Parkinson's disease, ALS, fragile X syndrome (FXS), Huntington disease, autosomal dominant spinocereberal ataxis (SCAs), spinal bulbar muscular atrophy (SBMA), correction of autosomal recessive genetic disorder that is caused by a deficiency in the expression or function of the Ataxia Telangiectasia Mutated (ATM) protein.

Embodiment 131: The synthetic exosome of embodiment 130, wherein said synthetic exosome contains a plasmid that encodes a class 2 CRISPR/Cas endonuclease and a guide RNA or a nucleic acid encoding a guide RNA, or said synthetic exosome contains a class 2 CRISPR/Cas endonuclease and a guide RNA or a nucleic acid encoding a guide RNA.

Embodiment 132: The synthetic exosome of embodiment 131, wherein said class 2 CRISPR/Cas endonuclease is a type II CRISPR/Cas endonuclease.

Embodiment 133: The synthetic exosome according to any one of embodiments 131-132, wherein the class 2 CRISPR/Cas endonuclease is a Cas9 polypeptide and the corresponding CRISPR/Cas guide RNA is a Cas9 guide RNA.

Embodiment 134: The synthetic exosome of embodiment 133, wherein said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

Embodiment 135: The synthetic exosome of embodiment 134, wherein said Cas9 protein comprises a Streptococcus pyogenes Cas9 protein (spCas9).

Embodiment 136: The synthetic exosome of embodiment 134, wherein said Cas9 protein comprises a Staphylococcus aureus Cas9 protein (saCas9).

Embodiment 137: The synthetic exosome of embodiment 134, wherein said Cas9 protein comprises a Streptococcus thermophilus Cas9 protein.

Embodiment 138: The synthetic exosome of embodiment 134, wherein said Cas9 protein comprises a Neisseria meningitides Cas9 protein (nmCas9).

Embodiment 139: The synthetic exosome of embodiment 134, wherein said Cas9 protein comprises a Treponema denticola Cas9 protein (tdCas9).

Embodiment 140: The synthetic exosome of embodiment 131, wherein the class 2 CRISPR/Cas endonuclease is a type V or type VI CRISPR/Cas endonuclease.

Embodiment 141: The synthetic exosome of embodiment 140, wherein the class 2 CRISPR/Cas endonuclease is selected from the group consisting of a Cpf1 polypeptide or a functional portion thereof, a C2c1 polypeptide or a functional portion thereof, a C2c3 polypeptide or a functional portion thereof, and a C2c2 polypeptide or a functional portion thereof.

Embodiment 142: The synthetic exosome of embodiment 141, wherein the class 2 CRISPR/Cas endonuclease comprises a Cpf1 polypeptide.

Embodiment 143: The synthetic exosome according to any one of embodiments 130-142, wherein said components of a CRISPR/Cas system are configured to produce insertions or deletions in ApoE4.

Embodiment 144: The synthetic exosome according to any one of embodiments 130-142, wherein said components of a CRISPR/Cas system are configured to replace ApoE4 with ApoE3 or ApoE2.

Embodiment 145: The synthetic exosome of embodiments 98-100, wherein said synthetic exosome contains an miRNA.

Embodiment 146: The synthetic exosome of embodiment 98-100, wherein said synthetic exosome contains an inhibitory RNA, or a nucleic acid encoding an inhibitory RNA.

Embodiment 147: The synthetic exosome of embodiment 146, wherein said exosome contains a DNA encoding an shRNA or an siRNA.

Embodiment 148: The synthetic exosome according to any one of embodiments 146-147, wherein said exosome contains an inhibitory RNA or a nucleic acid encoding an inhibitory RNA for the treatment of a neurodegenerative condition or a cancer.

Embodiment 149: The synthetic exosome of embodiment 148, wherein said exosome contains an inhibitory RNA or a nucleic acid encoding an inhibitory RNA for the treatment of a neurodegenerative condition selected from the group consisting of Alzheimer's disease, amyotrophic lateral sclerosis (ALS), Huntington's disease, and Parkinson's disease.

Embodiment 150: The synthetic exosome of embodiment 149, wherein said exosome contains an inhibitory RNA or a nucleic acid encoding an inhibitory RNA for the treatment of Alzheimer's disease.

Embodiment 151: The synthetic exosome of embodiment 150, wherein said inhibitory RNA inhibits expression of a target selected from the group consisting of a mutant APP (e.g., APPsw), and a mutant tau.

Embodiment 152: The synthetic exosome of embodiment 150, wherein said inhibitory RNA inhibits expression of a target selected from the group consisting of c-SCR, GGA3 adaptor protein, and acyl-coenzyme A cholesterol acyltransferase (ACAT-1).

Embodiment 153: A pharmaceutical formulation comprising:
a synthetic exosome according to any one of embodiments 1-152; and
a pharmaceutically acceptable carrier.

Embodiment 154: A kit comprising:
a container containing a nanoscale synthetic exosome according to any one of embodiments 1-152, and/or a pharmaceutical formulation according to embodiment 153;
and instructional materials teaching the use of said synthetic exosome to mitigate one or more symptoms associated with a disease characterized by amyloid deposits in the brain, and/or the use of said composition in delaying or preventing the onset of one or more of said symptoms.

Embodiment 155: A method of reducing the risk, lessening the severity, or delaying the progression or onset of a disease characterized by beta-amyloid deposits in the brain of a mammal, said method comprising:
administering, or causing to be administered, to said mammal synthetic exosome according to any one of embodiments 111-119 and 130-152, and/or a pharmaceutical formulation according to embodiment 153 in an amount sufficient to reducing the risk, lessen the severity, or delay the progression or onset of said disease.

Embodiment 156: The method of embodiment 155, wherein said disease is a disease selected from the group consisting of Alzheimer's disease, Cerebrovascular dementia, Parkinson's disease, Huntington's disease, Cerebral amyloid angiopathy, amyotrophic lateral sclerosis (ALS), traumatic brain injury (TBI), and stroke.

Embodiment 157: A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, said method comprising:
administering, or causing to be administered, to said mammal a synthetic exosome according to any one of embodiments 111-119 and 146-152, and/or a pharmaceutical formulation according to embodiment 153 in an amount sufficient to promote the processing of amyloid precursor protein (APP) by the non-amyloidogenic pathway and/or sufficient to reduce sAPPβ.

Embodiment 158: A method of promoting the processing of amyloid precursor protein (APP) by the non-amyloidogenic pathway as characterized by increasing sAPPα and/or the sAPPα/Aβ42 ratio in a mammal, said method comprising:
administering, or causing to be administered, to said mammal a synthetic exosome according to any one of embodiments 111-119 and 146-152, and/or a pharmaceutical formulation according to embodiment 153, wherein said administering is in an amount sufficient to promote the processing of amyloid precursor protein (APP) by the non-amyloidogenic pathway and/or sufficient to reduce sAPPβ.

Embodiment 159: A method of delivering one or more therapeutic moieties into the brain of a mammal, said method comprising:
administering, or causing to be administered, to said mammal an effective amount of a synthetic exosome according to any one of embodiments 1-97, wherein said exosome contains said one or more therapeutic moieties.

Embodiment 160: The method of embodiment 159 wherein said synthetic exosome comprises a synthetic exosome according to any one of embodiments 98-129.

Embodiment 161: The method of embodiment 159 wherein said one or more therapeutic moieties comprises components of a CRISPR/Cas system, components of a TALEN system, and/or components of a Zinc Finger protein.

Embodiment 162: The method of embodiment 161, wherein said exosome contains components of a CRISPR/Cas system for the treatment of Alzheimer's disease, Parkinson's disease, ALS, fragile X syndrome (FXS), Huntington disease, autosomal dominant spinocereberal ataxis (SCAs), spinal bulbar muscular atrophy (SBMA), correction of autosomal recessive genetic disorder that is caused by a deficiency in the expression or function of the Ataxia Telangiectasia Mutated (ATM) protein.

Embodiment 163: The method of embodiment 162, wherein said synthetic exosome comprises a synthetic exosome according to any one of embodiments 131-144.

Embodiment 164: The method according to any one of embodiments 159-163, wherein said mammal is a human.

Embodiment 165: The method according to any one of embodiments 159-163, wherein said mammal is a non-human mammal.

Embodiment 166: A method of treating a pathology in a mammal selected from the group consisting of Alzheimer's disease, Parkinson's disease, ALS, fragile X syndrome (FXS), Huntington disease, autosomal dominant spinocereberal ataxis (SCAs), spinal bulbar muscular atrophy (SBMA), an autosomal recessive genetic disorder that is caused by a deficiency in the expression or function of the Ataxia Telangiectasia Mutated (ATM) protein, said method comprising:
administering, or causing to be administered, to said mammal an effective amount of a synthetic exosome according to any one of embodiments 101-144.

Embodiment 167: The method of embodiment 166, wherein said mammal is a human.

Embodiment 168: The method of embodiment 166, wherein said mammal is a non-human mammal.

Embodiment 169: The method according to any one of embodiments 166-168, wherein said pathology is Alzheimer's disease.

Embodiment 170: The method of embodiment 169, wherein said synthetic exosome is a synthetic exosome according to any one of embodiments 130-144.

Embodiment 171: A microfluidic flow reactor for the synthesis of synthetic exosomes, said reactor comprising:
a central channel with two or more branch channels feeding said central channel and thereby forming a mixing junction, where the diameter of said central channel and branch channels and the angle provided between said central channel and branch channels are selected to maintain a backpressure of less than about 100 psi.

Embodiment 172: The microfluidic flow reactor of embodiment 171, where the diameter of said central channel and branch channels and the angle provided between said central channel and branch channels are selected to minimize flow turbulence.

Embodiment 173: The microfluidic flow reactor according to any one of embodiments 171-172 wherein the width of said central channel and/or branch channels independently range from about 0.5 μm or from about 1 μm, or from about 10 μm, or from about 20 μm, or from about 30 μm up to about 100 μm, or up to about 80 μm, or up to about 60 μm, or up to about 50 μm, or up to about 40 μm.

Embodiment 174: The microfluidic flow reactor according to any one of embodiments 171-173, wherein the height (depth) of said central channel and/or branch channels independently range from about 0.5 μm or from about 1 μm, or from about 10 μm, or from about 20 μm, or from about 30 μm up to about 100 μm, or up to about 80 μm, or up to about 60 μm, or up to about 50 μm, or up to about 40 μm.

Embodiment 175: The microfluidic flow reactor according to any one of embodiments 171-174 wherein the angle between said central channel and said lateral channels ranges from about 10 deg, or from about 15 deg, or from about 20 deg, or from about 25 deg up to about 90 deg, or up to about 80 deg, or up to about 70 deg, or up to about 60 deg, or up to about 50 deg.

Embodiment 176: The microfluidic flow reactor according to any one of embodiments 171-175, wherein said reactor comprises one or more pumps where said pumps provide a fluid pressure ranging from about 1 bar to about 30 bar.

Embodiment 177: The microfluidic flow reactor of embodiment 176, wherein said pumps provide a flow rate ranging from about 0.05 mL/min up to about 10 mL/min.

Embodiment 178: The microfluidic flow reactor according to any one of embodiments 171-177, wherein said reactor utilizes three independently regulated flow streams.

Embodiment 179: The microfluidic flow reactor of embodiment 178, where two flow streams comprise water and one flow stream comprises isopropyl alcohol.

Embodiment 180: The microfluidic flow reactor according to any one of embodiments 178-179, wherein the aqueous stream flow rate ranges from about 0.5 mL/min up to about 10 mL/min.

Embodiment 181: The microfluidic flow reactor according to any one of embodiments 178-180, wherein the aqueous stream flow rate ranges from about 0.5 mL/min up to about 10 mL/min.

Embodiment 182: The microfluidic flow reactor according to any one of embodiments 171-181, wherein said reactor comprises a pressure controller.

Embodiment 183: The microfluidic flow reactor according to any one of embodiments 171-182, wherein said reactor comprises a temperature controller (heater).

Embodiment 184: The microfluidic flow reactor according to any one of embodiments 171-183, wherein said reactor provides 1, 2, 3, 4, 5, or 6, or more mixing junctions.

Embodiment 185: The microfluidic flow reactor of embodiment 184, wherein said microfluidic flow reactor comprises a plurality of mixing junctions and contains functionalized lipids where said lipids are functionalized to react with a forming synthetic exosome at a second mixing junction.

Embodiment 186: The microfluidic flow reactor of embodiment 185, wherein said functionalized lipids comprise on ore more lipids selected from the group consisting of dioleoyl-sn-glycero-3-phosphocholine (N-aminoethyl), dioleoyl or dipalmitoyl-sn-glycero-3-phosphoethanolamine-N-[4-(p-maleimidomethyl)cyclohexane-carboxamide], dipalmitoyl-sn-glycero-3-phosphoethanolamine-N-[4-(p-maleimidophenyl)butyramide], dipalmitoyl-sn-Glycero-3-Phosphothioethanol, dipalmitoyl-sn-glycero-3-phosphoethanolamine-N-(succinyl), distearoyl-sn-glycero-3-phosphocholine (N-propynyl), dipalmitoyl-sn-glycero-3-phosphoethanolamine-N-dibenzocyclooctyl, and distearoyl-sn-glycero-3-phosphocholine (N-azidoethyl).

Embodiment 187: A method of making a synthetic exosome containing a therapeutic moiety, said method comprising: combining the components of a lipid bilayer as recited in any one of embodiments 1-92 and said therapeutic moiety in organic and aqueous phases in microchannels in a microfluidic flow reactor at a controlled flow ratio and pressure; and collecting the resulting samples comprising synthetic exosomes containing said therapeutic moiety.

Embodiment 188: The method of embodiment 187, wherein, said therapeutic moiety comprises a therapeutic moiety as recited in any one of embodiments 98-152.

Embodiment 189: The method according to any one of embodiments 187-188, wherein said method produces a synthetic exosome according to any one of embodiments 98-152.

Embodiment 190: The method according to any one of embodiments 187-189, wherein the samples are dialyzed to produce a dialyzed sample.

Embodiment 191: The method according to any one of embodiments 187-190, wherein the dialyzed sample is lyophilized to a powder.

Embodiment 192: The method according to any one of embodiments 171-191, wherein said microfluidic flow reactor comprises a microfluidic flow reactor according to any one of embodiments 171-186.

### Definitions

The term “about” when used with respect to a numerical value refers to that value ±10%, or ±5%, ±3%, or ±2%, or ±1% of that value. In certain embodiments about refers to ±10% of the value. In certain embodiments about refers to ±5% of the value. In certain embodiments about refers to ±2% of the value.

A receptor antagonist is a type of receptor ligand or drug that blocks or dampens agonist-mediated responses rather than provoking a biological response itself upon binding to a receptor. They are sometimes called blockers; examples include alpha blockers, beta blockers, and calcium channel blockers. In various embodiments receptor antagonists can comprise direct receptor antagonists, or allosteric receptor antagonists. Typically, direct antagonists have affinity but no little or no efficacy for their cognate receptors, and binding will typically disrupt the interaction and inhibit the function of an agonist or inverse agonist at their cognate receptor. Direct antagonists mediate their effects by binding to the active orthosteric (i.e., right place) site of a receptor (e.g., the binding site of the cognate ligand for that receptor).

An “allosteric antagonist” typically binds to other sites (than the native ligand (e.g., agonist) site) on the receptor or they may interact at unique binding sites not normally involved in the biological regulation of the receptor's activity.

The terms “subject,” “individual,” and “patient” may be used interchangeably and typically a mammal, in certain embodiments a human or a non-human primate. While the compositions and methods are described herein with respect to use in humans, they are also suitable for animal, e.g., veterinary use. Thus certain illustrative organisms include, but are not limited to humans, non-human primates, canines, equines, felines, porcines, ungulates, lagomorphs, and the like. Accordingly, certain embodiments contemplate the compositions and methods described herein for use with domesticated mammals (e.g., canine, feline, equine), laboratory mammals (e.g., mouse, rat, rabbit, hamster, guinea pig), and agricultural mammals (e.g., equine, bovine, porcine, ovine), and the like. The term “subject” does not require one to have any particular status with respect to a hospital, clinic, or research facility (e.g., as an admitted patient, a study participant, or the like). Accordingly, in various embodiments, the subject can be a human (e.g., adult male, adult female, adolescent male, adolescent female, male child, female child) under the care of a physician or other health worker in a hospital, psychiatric care facility, as an outpatient, or other, clinical context. In certain embodiments, the subject may not be under the care or prescription of a physician, or other, health worker. In certain embodiments the subject may not be under the care a physician or health worker and, in certain embodiments, may self-prescribe and/or self-administer the compounds described herein.

As used herein, the phrase “a subject in need thereof” refers to a subject, as described infra, that suffers or is at a risk of suffering (e.g., pre-disposed such as genetically pre-disposed) from the diseases or conditions listed herein.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease. In certain embodiments the prophylactically effective amount may be less than the therapeutically effective amount.

The terms “treatment,” “treating,” or “treat” as used herein, refer to actions that produce a desirable effect on the symptoms or pathology of a disease or condition, particularly those that can be effected utilizing the multi-component formulation(s) described herein, and may include, but are not limited to, even minimal changes or improvements in one or more measurable markers of the disease or condition being treated. Treatments also refers to delaying the onset of, retarding or reversing the progress of, reducing the severity of, or alleviating or preventing either the disease or condition to which the term applies, or one or more symptoms of such disease or condition. “Treatment,” “treating,” or “treat” does not necessarily indicate complete eradication or cure of the disease or condition, or associated symptoms thereof. In one embodiment, treatment comprises improvement of at least one symptom of a disease being treated. The improvement may be partial or complete. The subject receiving this treatment is any subject in need thereof. Exemplary markers of clinical improvement will be apparent to persons skilled in the art.

An “effective amount” refers to an amount effective, at dosages and for periods of time necessary, to achieve the desired therapeutic or prophylactic result. A “therapeutically effective amount” of an SE containing sAPPα and/or a comprising sAPPα or formulation thereof described herein may vary according to factors such as the disease state, age, sex, and weight of the individual, and the ability of the treatment to elicit a desired response in the individual. A therapeutically effective amount is also one in which any toxic or detrimental effects of a treatment are substantially absent or are outweighed by the therapeutically beneficial effects. The term “therapeutically effective amount” refers to an amount of one or more active agents described herein (e.g., a synthetic exosome (SE) containing sAPPα) or composition comprising the same that is effective to “treat” a disease or disorder in a mammal (e.g., a patient). In one embodiment, a therapeutically effective amount is an amount sufficient to improve at least one symptom associated with a neurological disorder, improve neurological function, improve cognition, or one or more markers of a neurological disease, or to enhance the efficacy of one or more pharmaceuticals administered for the treatment or prophylaxis of a neurodegenerative pathology. In certain embodiments, an effective amount is an amount sufficient alone, or in combination with a pharmaceutical agent to prevent advancement or the disease, delay progression, or to cause regression of a disease, or which is capable of reducing symptoms caused by the disease.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

As used herein, the phrases “improve at least one symptom” or “improve one or more symptoms” or equivalents thereof, refer to the reduction, elimination, or prevention of one or more symptoms of pathology or disease. Illustrative symptoms of pathologies treated, ameliorated, or prevented by the compositions (active agents) described herein (e.g., a SE containing sAPPα and/or a comprising sAPPα) include, but are not limited to, reduction, elimination, or prevention of one or more markers that are characteristic of the pathology or disease (e.g., of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, βAPPα/γAPPβ ratio, βAPPα/Aβ40 ratio, βAPPα/Aβ42 ratio, etc.) and/or reduction, stabilization or reversal of one or more diagnostic criteria (e.g., clinical dementia rating (CDR)). Illustrative measures for improved neurological function include, but are not limited to the use of the mini-mental state examination (MMSE) or Folstein test (a questionnaire test that is used to screen for cognitive impairment), the General Practitioner Assessment of Cognition (GPCOG), a brief screening test for cognitive impairment described by Brodaty et al., (2002) Geriatrics Society 50(3): 530-534, and the like.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Certain preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fvs (scFv), however, alternative expression strategies have also been successful. For example, Fab molecules can be displayed on phage if one of the chains (heavy or light) is fused to g3 capsid protein and the complementary chain exported to the periplasm as a soluble molecule. The two chains can be encoded on the same or on different replicons. The important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405 and 4,956,778). In various embodiments antibodies include all that have been displayed on phage (e.g., scFv, Fv, Fab and disulfide linked Fv) (Reiter et al. (1995) Protein Eng. 8: 1323-1331). In certain embodiments antibodies include, but are not limited to antibodies or antibody fragments selected from the group consisting of Fab, Fab′, Fab′-SH, F(ab′)₂, Fv, Fv′, Fd, Fd′, scFv, hsFv fragments, single-chain antibodies, cameloid antibodies, diabodies, and other fragments.

RNA interference (RNAi) therapeutics can result in prevention of a protein that plays a role in CNS disorders from being made. This can be achieved using complementary small interfering RNA, or siRNA, that are double-stranded molecules running 20-25 nucleotides in length. Similarly microRNA (miRNA) are small non-coding RNA molecule containing about 22 nucleotides and functions in RNA silencing and post-transcriptional regulation of gene expression that are beneficial in CNS disorders such as Alzheimer's disease.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration).

The phrase “cause to be administered” refers to the actions taken by a medical professional (e.g., a physician), or a person prescribing and/or controlling medical care of a subject, that control and/or determine, and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

### BRIEF DESCRIPTION OF THE DRAWINGS

FIG. 1 illustrates synthetic exosome synthesis, characterization, dialysis, and lyophilization. Panel A) Lipids (e.g., DPPC-1,2-dipalmitoyl-snglycero-3-phosphocholine, cholesterol, dihexadecyl phosphate (DCP); and (1-myristoyl-2-{P6-[(7-nitro-2-1,3-benzoxadizaol-4-yl)amino]helanoyl}-sj-glycero-3-phosphocholine) in IPA flow into an organic microfluidic reactor stream and cargo (if hydrophilic) in the aqueous stream. SEs are collected (panel B), characterized (panel C), dialyzed (panel D) and lyophilized for storage (panel E).

FIG. 2 illustrates atomic force microscopy (AFM) showing that the SE's can be deformed compared to conventional liposomes. Atomic Force Microscopy (AFM) shows conventional liposomes are not deformed. As illustrated, the SEs described herein are deformable and can penetrate tight junctions when compared to LPs.

FIG. 3, panels A-B, shows that sAPPα-SEs decrease sAPPβ and Aβ1-42 in vitro. sAPPα-SEs significantly decreased sAPPβ (panel A) and Aβ1-42 (panel B) in CHO-7W cells and were more effective than recombinant free sAPPα. Statistical analysis performed using ANOVA with Tukey's post-hoc analysis.

FIG. 4, panels A-B, shows the PK and biochemistry if IV sAPPα-SEs. Panel A) Human sAPPα peaked 1 hour after sAPPα-SE IVF deliver to wt mice and decreased to vehicle-only levels by 24 h (Stats: T-test). Panel B) sAPPβ levels are lower (but not significantly so) with sAPPα-SE delivery to EFAD huAPP-expressing mice.

FIG. 5 illustrates a 10-fold increase in brain levels of IDUA using SE-IDUA (−) particles compared to free IDUA.

FIG. 6 shows that SE-Cas9(−) has greater brain permeability than SE-Cas9 (+) particles.

FIG. 7 illustrates microreactor (microfluidic reactor) design. On top, the 26 μL reactor is shown and on the bottom the 10004 reactors.

FIG. 8 illustrates a custom reactor design. The design is based on minimizing turbulence in the mixing region.

FIG. 9 illustrates one embodiment of a flow chemistry machine (microfluidic reactor system).

FIG. 10 illustrates one embodiment of a microfluidic flow reactor design for SE-ligand reactions in series. The design is based on minimizing turbulence in the mixing join (mixing junction).

FIG. 11 illustrates hydrophobic small molecule encapsulation. The flow rate ratio versus size relationship is illustrated.

FIG. 12 illustrates encapsulation of a biologic. Illustrated is encapsulation of a protein with molecular weight 80 kDa.

FIG. 13 illustrates the synthetic exosomes with a cargo penetrating the blood brain barriers by squeezing through the tight junctions due to its deformability. We use a microfluidic based platform to encapsulate CNS biotherapeutic candidates in SEs. In certain embodiments, SEs for brain delivery are nanovesicles of <150 nm that are able to deform and cross the blood brain barrier while maintaining a cargo within. SEs, due to their deformability have the potential to cross the blood brain barrier (BBB) by physically squeezing between the tight junctions of the BBB.

FIG. 14, panels A-C, illustrates a synthetic scheme, illustrative moieties for decorating synthetic exosomes (SEs), and an illustrative strategy for decoration of SE with peptides for enhanced brain permeability. A) Illustrative peptides for decorating synthetic exosomes (SEs). Panel B) Illustrative lipids for decorating synthetic exosomes (SEs). Panel C) Illustrative synthetic scheme for decorating SEs with peptides using a “click” chemistry.

### DETAILED DESCRIPTION

This disclosure pertains to the development of a linkage-free brain delivery platform to encapsulate CNS therapeutic candidates in Synthetic Exosomes (SEs). In various embodiments the SEs for brain delivery are liposomes of less than about 200 nm average (ore median) diameter that are able to deform while retaining a cargo within.

Without being bound to a particular theory, it is believed the synthetic exosomes are able to cross the blood-brain barrier (BBB) by physically squeezing between the tight junctions of the endothelial cells lining brain capillaries, as well as by the astrocytic projections (‘feet’) that also comprise the BBB, like miniature cells while protecting the therapeutic cargo encapsulated within the exosome.

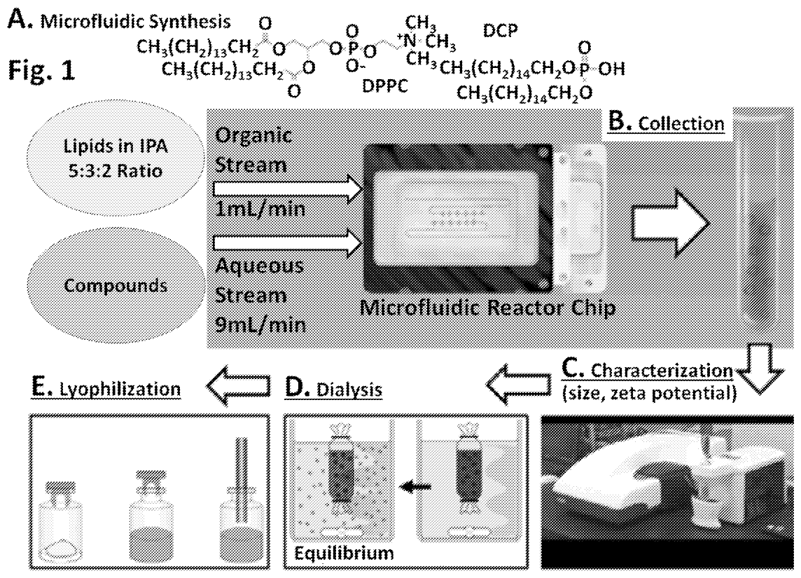

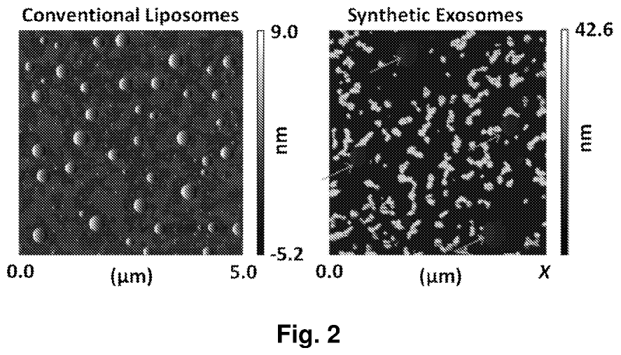

In various embodiments the therapeutic-loaded SEs are synthesized using a microfluidic flow reactor (see, e.g., FIG. 1). The SEs thus produced can be stored as a lyophilized powder for months without any loss of the drug cargo. In various embodiments the synthetic exosomes are composed of GRAS (generally regarded as safe) materials, and are able to encapsulate a variety of molecules including small molecules—both lipophilic and hydrophilic—DNA/RNA/siRNA, as well as peptides, proteins, aptamers, and combinations thereof. A variety of lipids such as DPPC (1,2-dipalmitoyl-snglycero-3-phosphocholine), cholesterol, and DCP (dihexadecyl phosphate) can be used in predetermined ratios to generate SEs with the desired properties including deformability.

The flow rates of the organic phase (typically carrying the lipids and lipophilic compounds) in, e.g., IPA (isopropyl alcohol) and aqueous phase (typically carrying the potential hydrophilic therapeutic molecules) can be finely controlled to yield SEs with specific size (60<φ<500 nm), zeta potential (-50<ξ<50) and deformability.

If desired the surface of the synthetic exosomes can be modified to include different surface charges, using a variety of molecules like PEG for longer circulatory half-life, and carrier proteins for targeted therapeutic delivery.

Having been optimized (as illustrated herein), the microfluidic synthesis of SEs is readily scalable for obtaining larger amounts and allows good batch-to-batch reproducibility.

As proof of principle, we encapsulated fluorescently-labeled zoledronate for the purpose of transdermal delivery to a local site (calvarial skin of the skull) (see, e.g., U.S. Patent Application No: WO 2017087685 (PCT/US2016/062552)). In addition to presenting the size, encapsulation efficiency, zeta potential and tunability of SEs synthesized by this method, we also provided evidence of the successful long-term storage of the drug-loaded SEs. Using high-resolution fluorescent and confocal imaging. we showed the successful transdermal delivery of the encapsulated payload. The delivery was greater for SEs as compared to non-deformable nanovesicles or aqueous drug solutions. While this study was not performed for the purpose of delivery of the brain, it provides proof-of-concept for the use of SEs for drug delivery across a membrane. The tunability of our microfluidic SE synthesis method allows for modulation of the therapeutic-loaded SE size.

In our ongoing studies to achieve successful delivery of a potential therapeutic to the brain, we encapsulated a large protein fragment, the neurotrophic factor soluble Amyloid Precursor Protein alpha (sAPPα, a 678 amino acids in length) and characterized the sAPPα-SEs for size, encapsulation efficiency and zeta potential (see Table 1).

TABLE 1
Properties of sAPPα-SE.

| | Characteristics | | |
|---|---|---|---|
| | Zeta Potential | Size | Encapsulation |
| SE | (mV) | (nm) | Efficiency (%) |
| sAPPα-SE | −40 | 80-120 | >30 |

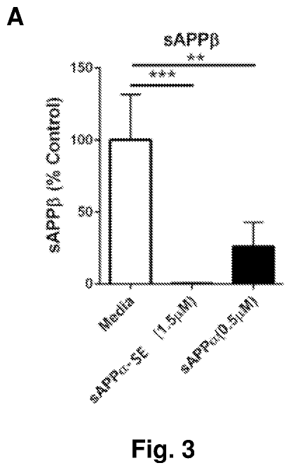

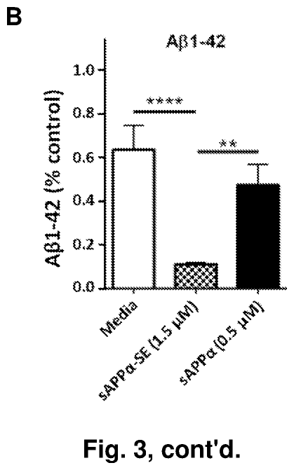

Next, to establish that we could achieve successful delivery and release of the SE payload to cells and maintain the biological activity of the cargo, we tested sAPPα-SEs in vitro in cells. sAPPα is an endogenous inhibitor of BACE1 (beta-site cleaving enzyme 1), the enzyme responsible for cleavage of full-length (FL) APP resulting in production of sAPPβ and the β C-terminal fragment (βCTF). βCTF is then cleaved by the γ secretase complex to produce amyloid-β (Aβ) and the APP intracellular domain. Therefore, successful delivery of sAPPα in cells that express FL APP should result in a decrease in sAPPβ and βCTF and, because of the decrease in the latter, a decrease in Aβ. As shown in FIG. 3, panel A, 48 hours after delivery of free recombinant unencapsulated sAPPα or sAPPα-SEs to Chinese Hamster Ovary cells that stably express human FL APP (CHO-7W cells), we found sAPPα-SEs more significantly decreased sAPPβ as compared to the media only control than free sAPPα. FIG. 3, panel B shows the decrease in Aβ1-42 is significantly greater with sAPPα-SEs than free sAPPα. The greater effect of sAPPα-SEs may be due to protection of sAPPα in the SEs from metabolism or other degradation and/or more efficient delivery of the sAPPα to the target for interaction, the enzyme BACE1.

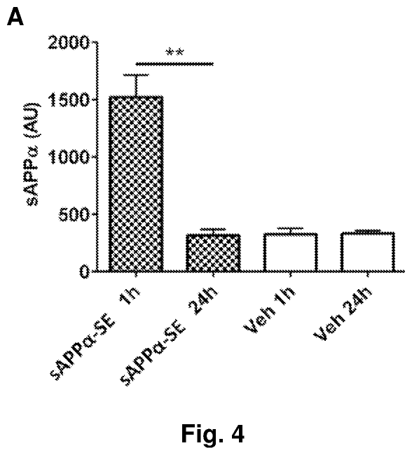

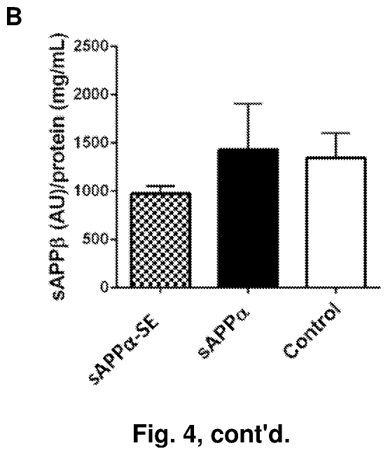

We next determined if peripheral injection of sAPPα-SEs would result in successful delivery of sAPPα to the brain of mice. To distinguish endogenously expressed sAPPα from the exogenous SE-encapsulated sAPPα, we used wildtype mice expressing only endogenous mouse APP and injected SEs loaded with recombinant human sAPPα; we then determined human sAPPα levels in brain using a human-sAPPα-specific AlphaLISA (Perkin-Elmer). As shown in FIG. 4, panel A, 1 hour after IV delivery of sAPPα-SEs, levels of human sAPPα in mouse brain tissue were 1500 (AU) as compared to only ˜300 (AU) at 24 hours, considered the background level since values were similar for the vehicle-only 1 and 24 hour controls. Assay of known concentrations of sAPPα allowed us to determine the concentration, which was found to be a pharmacological relevant concentration of ˜12 nM. These results indicate successful brain delivery of potentially efficacious levels of sAPPα to the brain.

Finally, to ascertain target engagement, we injected sAPPα-SEs into EFAD Alzheimer's disease (AD) model mice. EFAD transgenic mice are an AD model that express human apolipoprotein E4 (E) as well as human APP with three familial AD mutations and human presenilin 1 with two familial AD mutations (FAD); as a result, these mice show AD-like amyloid pathology at an early age, starting with increased production of sAPPβ. As shown in FIG. 4, panel B, sAPPβ was only lower than vehicle-only control with sAPPα-SEs and not free sAPPα. This indicates target engagement and preservation of biological activity of sAPPα encapsulated in SEs. These data support the efficacy of sAPPα-SE delivery and the engagement of the target in the brain.

In view of the foregoing, described herein are improved microfluidic flow reactors for the fabrication of synthetic exosomes. Also provide are improved exosome formulations that are believed to provide improved delivery to the central nervous system and that provide particular zeta potentials as desired. Additionally the improved exosomes are stable in solution (do not substantially aggregate) and effectively retain loaded therapeutic moieties thereby providing effective delivery across the blood brain barrier (BBB).

### Improved Synthetic Exosome Formulations.

In various embodiments the synthetic exosomes described herein comprise a lipsome formed from a lipid a lipid bilayer, where the lipid bilayer comprises or consists of:
A) one or more phospholipids selected from the group consisting of phosphate lipids, phosphoglycerol lipids, phosphocholine lipids, and phosphoethanolamine lipids where the lipid carbon chain ranges from 3 to 24 carbon atoms;
B) cholesterol, cholesterol hemicsuccinate, or a phytosterol; and
C_a non-ionic surfactant.

In certain embodiments the synthetic exosome ranges in size from about 50 nm up to about 200 nm in diameter. Typically, the synthetic exosome is capable of crossing the blood brain barrier without substantially leaking said therapeutic moiety. In certain embodiments the exosome is capable of crossing the blood/brain barrier (BBB) and delivering a therapeutic moiety contained therein to the central nervous system without losing more than about 40%, or without losing more than 30%, or without losing more than 20%, or without losing more than 10%, or without losing more than 5%, or without losing more than 3%, or without losing more than 1% of a therapeutic moiety contained therein.

In certain embodiments the lipid bilayer comprising the synthetic exosome consists of more phospholipids (e.g., 1, 2, 3, 4, or more phospholipids), cholesterol and/or and/or cholesterol hemisuccinate, and/or a phytosterol; and a non-ionic surfactant.

In certain embodiments the lipid bilayer comprising the synthetic exosome does not contain an alcohol (e.g., ethanol). In certain embodiments the lipid bilayer comprising the synthetic exosome does not contain glutathione-maleimide-PEG2000-distearoyl phosphatidyl ethanolamine In various embodiments the synthetic exosome is not a transferosome or an ethosome.

In certain embodiments the molar ratio of total phospholipid to cholesterol, cholesterol hemisuccinate, and/or phytosterol ranges from about 4-8 moles of phospholipids] to about 1-2 moles of cholesterol.

In certain embodiments the amount of surfactant ranges from about 1%, or from about 3%, or from about 5%, or from about 8% up to about 18%, or up to about 15%, or up to about 13%, or up to about 10% (wt/wt). In certain embodiments the surfactant comprise one or more surfactants selected from the group consisting of Span 80, Tween 20, BRIJ® 76 (stearyl poly(10)oxy ethylene ether), BRIJ® 78 (stearyl poly(20)oxyethylene ether), BRIJ® 96 (oleyl poly(10)oxy ethylene ether), and BRIJ® 721 (stearyl poly (21) oxyethylene ether). In various embodiments the surfactant comprises or consists of Span 80.

In certain embodiments the synthetic exosome lipid bilayer (LB) comprises about 10% to about 20%, or about 15% Span 80 by weight.

Phospholipids

In various embodiments the lipid bilayer comprising the synthetic exosomes described herein is formed from 1, 2, 3, or 4, or more phospholipids, cholesterol or a functionalized cholesterol (e.g., cholesterol hemisuccinate (CHEMS), lysine-based cholesterol (CHLYS), and PEGylated cholesterol (Chol-PEG), or a phytosterol, and one or more surfactants (e.g., Span-80).

In certain embodiments the synthetic exosomes bear one or more targeting moieties attached to the lipid bilayer. Illustrative targeting moieties include, but are not limited to amino acids (e.g., amino acid functionalized lipids) that are transported into the central nervous system (CNS) by amino acid transporters, transferrin folic acid, various antibodies, CD171, and the like.

In various embodiments the lipid bilayer comprises one or more phospholipids, including, but not limited to phosphate, phosphoglycerol and phosphocholine lipids, phosphoethanolamine lipids with/without polyethylene glycol (7-100 monomers), and cholesterol. In various embodiments the lipid carbon chain ranges from about 3 to about 24 carbons atoms. Suitable phospholipids for use in the synthetic exosomes described herein can include Dihexanoyl-sn-glycero-3-phosphate (DHPA), Didecanoyl-sn-glycero-3-phosphate (DDPA), Distearoyl-sn-glycero-3-phosphate (DTPA), Dihexadecyl phosphate (DHP), and the like.

Phosphoglycerol lipids suitable for use in the lipid bilayer comprising the synthetic exosomes described herein can include Dihexanoyl-sn-glycero-3-phospho-(1′-rac-glycerol) (DHPG), Dilauroyl-sn-glycero-3-phospho-(1′-rac-glycerol) (DLPG), Distearoyl-sn-glycero-3-phospho-(1′-rac-glycerol) (DTPG), and the like.

Phosphocholine lipids suitable for use in the lipid bilayer comprising the synthetic exosomes described herein can include Dipropionyl-sn-glycero-3-phosphocholine (PC), Diheptanoyl-sn-glycero-3-phosphocholine (DHPC), Dimyristoyl-sn-glycero-3-phosphocholine (DMPC), Dilignoceroyl-sn-glycero-3-phosphocholine (DGPC), and the like.

Phospholipids with an alkyne such as phosphoethanolamine suitable for use in the lipid bilayer comprising the synthetic exosomes described herein can include Dihexanoyl-sn-glycero-3-phosphoethanolamine (DHPE), Distearoyl-sn-glycero-3-phosphoethanolamine (DTPE), and the like.

Phosphoethanolamine-PEG lipids suitable for use in the lipid bilayer comprising the synthetic exosomes described herein can include Dipalmitoyl-sn-glycero-3-phospho(ethylene glycol) (DPPEG1), Dimyristoyl-sn-glycero-3-phosphoethanolamine-N-[methoxy(polyethylene glycol)-350 (DMPEG350), Distearoyl-sn-glycero-3-phosphoethanolamine-N-[methoxy(polyethylene glycol)-350] (DTPEG350), Dimyristoyl-sn-glycero-3-phosphoethanolamine-N-[methoxy(polyethylene glycol)-550] (DMPEG550), Dimyristoyl-sn-glycero-3-phosphoethanolamine-N-[methoxy(polyethylene glycol)-1000] (DMPEG1000).

Other lipids suitable for use in the lipid bilayer comprising the synthetic exosomes described herein can include Dioleoyl-sn-glycero-3-phosphocholine (N-aminoethyl) (PC-NH2), Diphytanoyl-sn-glycero-3-phosphoethanolamine, Dioleoyl-3-trimethylammonium-propane (DOTAP), Distearoyl-3-trimethylammonium-propane (DSTAP), Dimyristoyl-3-trimethylammonium-propane (DMTAP), Di-O-octadecyl-sn-glycero-3-phosphocholin (DOPC), and the like.

Cholesterol and Cholesterol Analogs/Derivatives

In various embodiments the lipid bilayer comprising eth synthetic exosomes described herein contains cholesterol, a cholesterol derivative, or a cholesterol analog (e.g., a phytosterol).

Illustrative cholesterol derivatives include, but are not limited to cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, Hederoside, and the like. In certain embodiments the cholesterol derivative is cholesterol hemisuccinate.

In certain embodiments a phytosterol is used in addition to cholesterol, or in place of cholesterol. Suitable phytosterols include, but are not limited to 9,10-secosteroids (e.g., vitamin D3, vitamin D2, calcipotriol, and the like), C-24 alkyl steroids (e.g., stigmasterol, β-sitosterol, and the like), and pentacyclic steroids (e.g., betulin, lupeol, ursolic acid, and oleanolic acid). In certain embodiments the phytosterol is a C-24 alkyl steroid.

It will be recognized that whenever cholesterol (CHOL) is described herein the cholesterol, cholesterol derivative, or phytocholesterol is further functionalized. Thus, for example, in certain embodiments, the cholesterol, cholesterol derivative, or phytocholesterol is pegylated.

Particular Effective Synthetic Exosome Formulations.

The particular lipid mixture used for a synthetic exosome is determined by consideration of the nature of the molecule(s) to be entrapped, the anatomical delivery location and the desire surface charge. For example, for biological molecules (e.g., proteins, nucleic acids, antibodies, and the like) a mixture of 2-4 lipids with small carbon chains (e.g., 3-14 carbon atoms), are used in combination with cholesterol, and/or functionalized cholesterol, and/or phytosterol, and a surfactant (e.g., Span-80) are utilized to form the exosome. In various embodiments illustrative, but non-limiting embodiments, the concentration of surfactant (e.g., Span-80) can range from about 1%, or from about 5% up to about 20%, or up to about 15% (w/w) depending on the degree of deformability needed. Illustrative formulations particularly well suited for delivery of biologic payloads are shown in Table 2.

TABLE 2
Illustrative synthetic exosome formulations for biologics.

| Zeta | Lipid mixtures (in equimolar) so |
|---|---|
| Potential (ζ) | ratios below are molar ratios. |
| (mV) | Example for biologics |
| −20 ≤ ζ | 3:2:1 (DHPA:DHP:CH) |
| | 1:5:1 (DHPG:DHPA:CH) |
| | 2:5:1:2 (DHPG:DHPA:PC—NH2:CH) |
| | 2:4:1:1:2 (DHPG:DHPA:PC—NH2:DMPEG350:CH) |
| −20 ≤ ζ ≤ 20 | 2:2:1 (DHPG:DHPC:CH) |
| | 4:4:1:2 (DHPG:DHPA:PC—NH2:CH) |
| | 2:2:1 (DHPG:DHPA:CH) |
| | 4:4:1:1:2 (DHPG:DHPA:PC—NH2:DMPEG550:CH) |
| | 2:2:1 (DHPG:DTPE:CH) |
| | 2:2:1 (DHPG:DMTAP:CH) |
| | 4:4:1:2 (DHPG:DMTAP:PC—NH2:CH) |
| | 4:4:1:1:2 (DHPG:DMTAP:PC—NH2:DMPEG550:CH) |
| Z > 20 | 2:4:1 (DHPC:DTPE:CH) |
| | 2:4:1 (DHPC:DOTAP:CH) |
| | 2:4:1:2 (DHPC:DMTAP:PC—NH2:CH) |
| | 2:4:1:1:2 (DHPC:DMTAP:PC—NH2:DMPEG350:CH) |

*In certain embodiments, CH is cholesterol. In certain embodiments, CH is a cholesterol derivative (e.g., CHEMS, CHLYS, Chol-PEG, and the like). In certain embodiments CH is a phytosterol (e.g., 9,10-secosteroids (e.g., vitamin D3, vitamin D2, calcipotriol, and the like), C-24 alkyl steroids (e.g., stigmasterol, β-sitosterol, and the like), and pentacyclic steroids (e.g., betulin, lupeol, ursolic acid, and oleanolic acid).

For delivery of hydrophobic small molecules a mixture of 2-4 lipids with large carbon chains (e.g., 14-24 carbon atoms), are used in combination with cholesterol, and/or functionalized cholesterol, and/or phytosterol, and a surfactant (e.g., Span-80) are utilized to form the exosome. Illustrative formulations particularly well suited for delivery of small molecule are shown in Table 3.

TABLE 3
Illustrative synthetic exosome formulations for biologics.

| Zeta | Lipid mixtures (in equimolar) so |
|---|---|
| Potential (ζ) | ratios below are molar ratios. |
| (mV) | Example for biologics |
| −20 < ζ | 4:2:1 (DTPA:DHP:CH) |
| | 1:5:1 (DTPG:DTPA:CH) |
| | 1:5:1:2 (DTPG:DTPA:PC—NH2:CH) |
| | 1:4:1:1:2 (DTPG:DTPA:PC—NH2:DMPEG350:CH) |
| −20 ≤ ζ ≤ 20 | 2:2:1 (DTPG:DGPC:CH) |
| | 4:4:1:2 (DTPG:DDPA:PC—NH2:CH) |
| | 2:2:1 (DTPG:DDPA:CH) |
| | 4:4:1:1:2 (DTPG:DDPA:PC—NH2:DMPEG550:CH) |
| | 2:2:1 (DTPG:DTPE:CH) |
| | 2:2:1 (DTPG:DMTAP:CH) |
| | 4:4:1:2 (DTPG:DMTAP:PC—NH2:CH) |
| | 4:4:1:1:2 (DTPG:DMTAP:PC—NH2:DMPEG550:CH) |
| Z > 20 | 2:4:1 (DMPC:DTPE:CH) |
| | 2:4:1 (DMPC:DOTAP:CH) |
| | 2:4:1:2 (DMPC:DMTAP:PC—NH2:CH) |
| | 2:4:1:1:2 (DMPC:DMTAP:PC—NH2:DMPEG350:CH) |

*In certain embodiments, CH is cholesterol. In certain embodiments, CH is a cholesterol derivative (e.g., CHEMS, CHLYS, Chol-PEG, and the like). In certain embodiments CH is a phytosterol (e.g., 9,10-secosteroids (e.g., vitamin D3, vitamin D2, calcipotriol, and the like), C-24 alkyl steroids (e.g., stigmasterol, β-sitosterol, and the like), and pentacyclic steroids (e.g., betulin, lupeol, ursolic acid, and oleanolic acid).

In certain embodiments, CH in the formulations in Tables 2 and 3 is cholesterol.

In certain embodiments, CH in the formulations in Tables 2 and 3 is a cholesterol derivative. Thus, in certain embodiments, CH in the formulations in Tables 2 and 3 is cholesterol hemisuccinate. In certain embodiments, CH in the formulations in Tables 2 and 3 is lysine-based cholesterol (CHLYS). In certain embodiments, CH in the formulations in Tables 2 and 3 is 20-hydroxychloesterol. In certain embodiments, CH in the formulations in Tables 2 and 3 is 22-hydroxycholesterol. In certain embodiments, CH in the formulations in Tables 2 and 3 is 24-hydroxycholesterol. In certain embodiments, CH in the formulations in Tables 2 and 3 is 25-hydroxy cholesterol. In certain embodiments, CH in the formulations in Tables 2 and 3 is 27-hydroxycholesterol. In certain embodiments, CH in the formulations in Tables 2 and 3 is cholesteryl succinate. In certain embodiments, CH in the formulations in Tables 2 and 3 is cholic succinate. In certain embodiments, CH in the formulations in Tables 2 and 3 is cholic tri-succinate. In certain embodiments, CH in the formulations in Tables 2 and 3 is lithocholic succinate. In certain embodiments, CH in the formulations in Tables 2 and 3 is chenodesoxycholic bis-scuccinate. In certain embodiments, CH in the formulations in Tables 2 and 3 is Hederoside.

In certain embodiments, CH in the formulations in Tables 2 and 3 is a phytosterol. Thus, in certain embodiments CH in the formulations in Tables 2 and 3 is a 9,10-secosteroids. In certain embodiments CH in the formulations in Tables 2 and 3 is vitamin D3. In certain embodiments CH in the formulations in Tables 2 and 3 is vitamin D2. In certain embodiments CH in the formulations in Tables 2 and 3 is calcipotriol. In certain embodiments CH in the formulations in Tables 2 and 3 is a C-24 alkyl steroid. In certain embodiments CH in the formulations in Tables 2 and 3 is stigmasterol. In certain embodiments CH in the formulations in Tables 2 and 3 is β-sitosterol. In certain embodiments CH in the formulations in Tables 2 and 3 is a pentacyclic steroid. In certain embodiments CH in the formulations in Tables 2 and 3 is betulin. In certain embodiments CH in the formulations in Tables 2 and 3 is lupeol. In certain embodiments CH in the formulations in Tables 2 and 3 is ursolic acid. In certain embodiments CH in the formulations in Tables 2 and 3 is oleanolic acid.

The foregoing formulations are illustrative and non-limiting. Using the teaching provided herein, numerous other exosome formulations will be available to one of skill in the art.

### Targeting Moieties

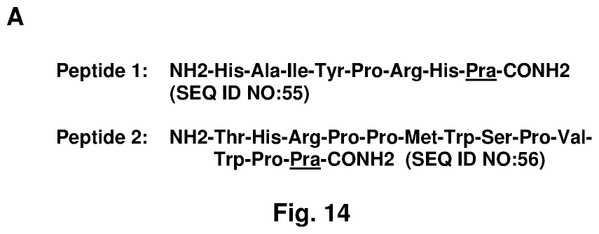

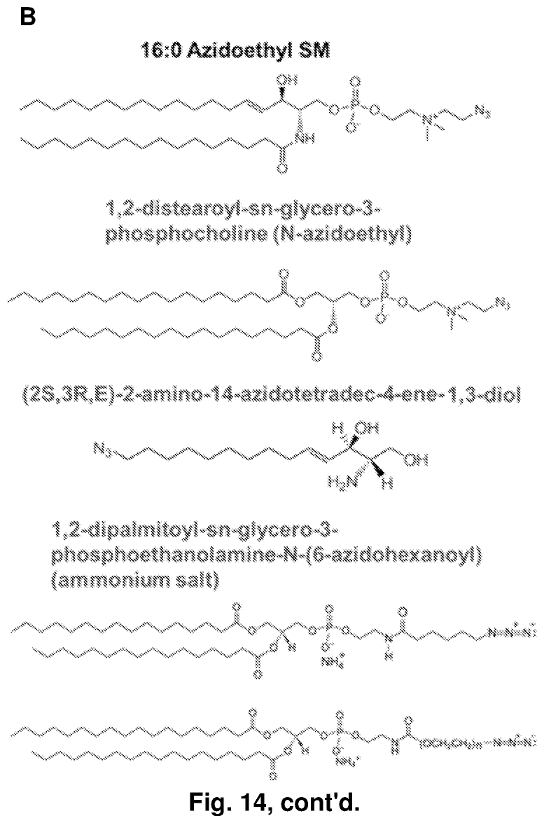

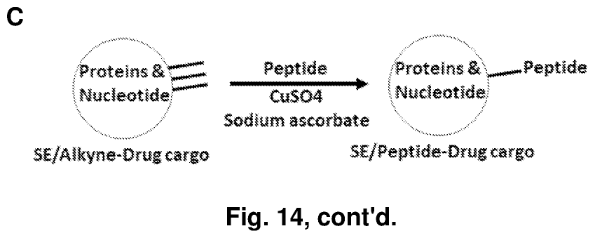

In certain embodiments the synthetic exosomes bear one or more targeting moieties attached to the lipid bilayer. Illustrative targeting moieties include, but are not limited to amino acids (e.g., amino acid functionalized lipids) that are transported into the central nervous system (CNS) by amino acid transporters, transferrin (e.g., transferrin functionalized lipids), transferrin receptor binding peptides (see, e.g., FIG. 14, panel A), folic acid, various BBB endothelial cell binding antibodies, CD171, and the like.

In certain embodiments the phospholipid or cholesterol comprising the synthetic exosomes described herein can be functionalized to thereby attach one or more targeting moieties. In certain embodiments the targeting moieties can comprise an amino acid to exploit amino acid transporters for internalization into a target cell. Essential amino acids are commonly transported across the BBB through specific transporters to participate in brain amino acid metabolism, such as the synthesis of neurotransmitters. Based on the difference of the substrates, amino acid transporters are divided into cationic, anionic, and neutral amino acid transporters. Large neutral amino acid transporter (LAT1) is the most abundant carrier for amino acids, which is expressed on both luminal and abluminal membranes of BCECs. LAT1 carries large neutral amino acids such as leucine, tryptophan, tyrosine, and phenylalanine across the BBB in the ion-independent pathway. Accordingly, in certain embodiments the targeting moiety can comprise carries a large neutral amino acid such as leucine, tryptophan, tyrosine, and phenylalanine.

Other illustrative targeting moieties include, but are not limited to antibodies, lectins, transferrin, folic acid, CD171, and the like.

### Synthetic Exosome (SE) Synthesis Using Microfluidic Flow Reactors.

In one illustrative embodiment a microfluidic reactor is used to synthesize the synthetic exosomes. In certain embodiments the microfluidic reactors uses 3 pumps to flow 3 fluids into the microfluidic chip. In certain embodiments, two of the streams are water and the other stream is isopropyl alcohol (IPA).

In certain embodiments the aqueous stream flow rate can range from 0.5 mL/min-10 mL/min, depending on the particle size desire and contains any biologics if needed. Each one of the flow rates can be manipulated individually.

In certain embodiments the organic stream flow rate can range from 0.05 mL/min-5 mL/min, depending on the particle size desire and contains the lipids mixture and any hydrophobic small molecule if needed.

In certain embodiments the microflow fluidic flow reactor utilizes one or more organic stream that contain components of the lipid bilayer (e.g., cholesterol, phospholipid, surfactant), and one or more aqueous (e.g., water) streams. In certain embodiments the organic stream comprise an alcohol (e.g., isopropyl alcohol) in addition to the components of the lipid bilayer.

In various embodiments the therapeutic moiety (cargo) is provided in the stream that is most likely to suspend or dissolve the moiety. Thus, for example, in typical embodiments, a hydrophobic therapeutic moiety is provided in the organic stream, while hydrophilic moieties are provided in the aqueous stream. Thus by way of illustration small organic molecules (e.g., hydrophobic small organic molecules) will be provided in the organic stream. Hydrophilic moieties such as peptides, enzymes, proteins and antibodies, nucleotides, DNA, and the like can be provided in the aqueous stream. It will be recognized that, in certain embodiments, two, three, or four different therapeutic moieties can be loaded into each synthetic exosome.

In certain illustrative, but non-limiting embodiments, the organic stream lipid mixture concentration ranges from about 5 mM to about 20 mM and any hydro phobic cargo will range from 0.05 mM up to about 2 mM depending on the cargo solubility. In certain illustrative, but non-limiting embodiments, ff the aqueous stream contains a hydrophilic molecule its concentration ranges from about 0.01 mg/mL to about 5 mg/mL depending on its solubility.

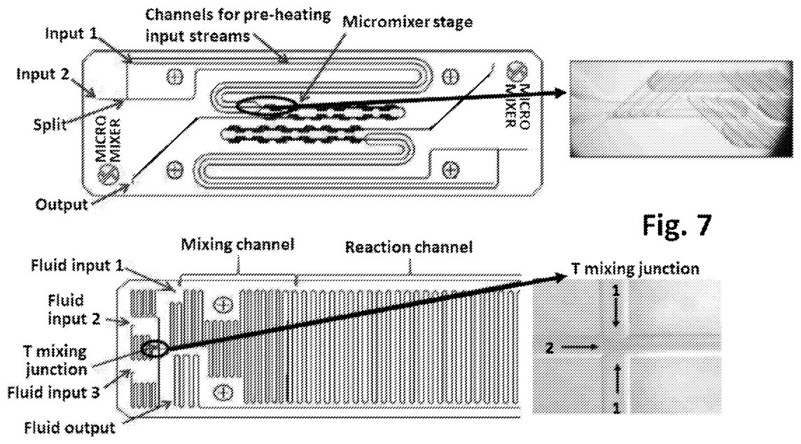

For the synthetic exosome synthesis, we have used two commercially available reactors: a 264 and the 10004 reactors but other reactors are also possible. The design of two illustrative reactors is shown in FIG. 7.

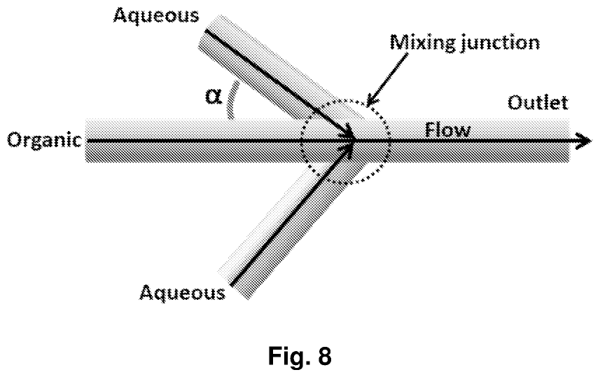

In order to improve the SE synthesis, we can use custom made reactors, their design is shown in FIG. 8. As shown in the illustrative embodiment shown in FIG. 8, the microfluidic flow reactor comprises a central channel with two or more branch channels feeding the central channel and thereby forming a mixing junction, where the diameter of said central channel and branch channels and the angle provided between said central channel and branch channels are selected to maintain a backpressure of less than about 100 psi. The design is based on minimizing turbulence in the junction while maintaining backpressure of less than 100 psi, the angle of impact in the mixing junction allows us to minimize turbulence while the channel width and height allow us to control the pressure.

In certain embodiments the width(s) of said central channel and/or branch channels independently range from about 0.5 μm or from about 1 μm, or from about 10 μm, or from about 20 μm, or from about 30 μm up to about 100 μm, or up to about 80 μm, or up to about 60 μm, or up to about 50 μm, or up to about 40 μm. In certain embodiments the height(s) (depth(s)) of the central channel and/or branch channels independently range from about 0.5 μm or from about 1 μm, or from about 10 μm, or from about 20 μm, or from about 30 μm up to about 100 μm, or up to about 80 μm, or up to about 60 μm, or up to about 50 μm, or up to about 40 μm. In certain embodiments the angle (a) between the central channel and said lateral channels ranges from about 10°, or from about 15°, or from about 20°, or from about 25° up to about 90°, or up to about 80°, or up to about 70°, or up to about 60°, or up to about 50°. In certain embodiments the reactor comprises one or more pumps where said pumps provide a fluid pressure ranging from about 1 bar to about 31 bar.

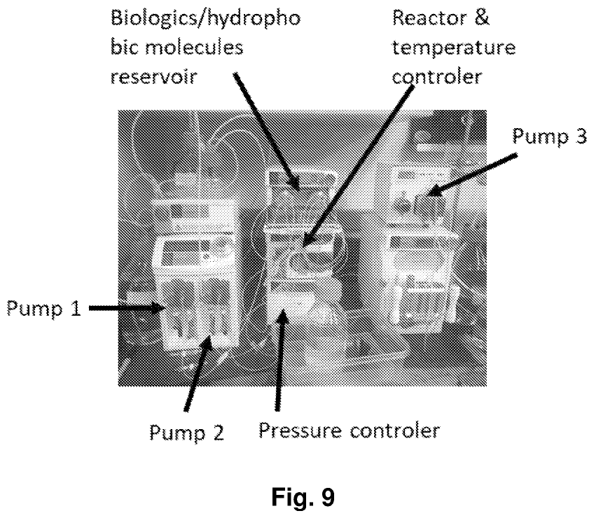

One embodiment of the microfluidic reactor system is shown in FIG. 9 and its elements are highlighted.

Functionalization of the Lipids in Series

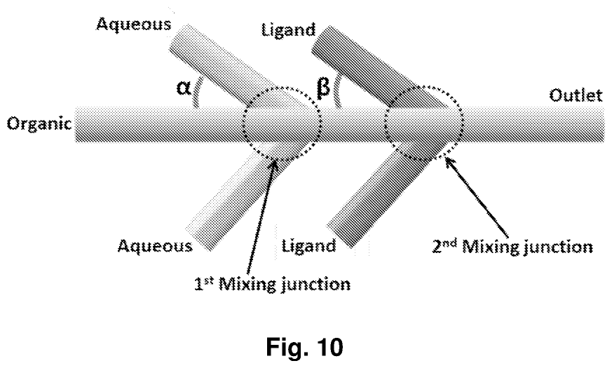

Synthetic exosome synthesis using the microfluidic reactor also enables us to use functionalized lipids such as the one listed in Table 4 below to tag different ligands to the synthetic exosome in series. This can be achieved with the use of reactors connected in series or with the design of the new reactor shown in FIG. 10. As shown in the illustrative embodiment shown in FIG. 10, the microfluidic flow reactor comprises a central channel with two or more branch channels feeding the central channel and thereby forming a first mixing junction, and a second set of branch channels feeding the central channel and thereby forming a second mixing junction. It will be recognized that the reactor can contain additional mixing junctions. Thus, reactors comprising 2, 3, 4, 5, 6 or more mixing junctions are contemplated. In certain embodiments the diameter of the central channel and branch channels and the angle provided between the central channel and branch channels are selected to maintain a backpressure of less than about 100 psi. Again, the design is based on minimizing turbulence in the junction while maintaining backpressure of less than 100 psi, the angle of impact in the mixing junction allows us to minimize turbulence while the channel width and height allow us to control the pressure.

For the synthesis, after the SE are form in the first mixing junction, then the ligand can react with the SE in the second mixing junction. Additionally, in certain embodiments more tagged lipids can be reacted with the forming SE in additional mixing junctions. All of the reactions can be independently tuned.

In certain illustrative, but non-limiting embodiments the “tagged” synthesis facilitates the use of specific proteins ligands that can include, but are not limited to, ligands that will have a receptor in the BBB. Illustrative ligands include, but are not limited to insulin, transferrin, low density lipoprotein receptor-related protein 1, or any other ligand such as an amino acid.

In certain embodiments one of skill can use the lipid NHS ester and then functionalize it with various ligands such as peptide, proteins, amino acids, etc.

TABLE 4
Illustrated functionalized lipids can be purchased
or prepared by “tagged” synthesis.

Functionalized Lipid
Dioleoyl-sn-glycero-3-phosphocholine (N-aminoethyl)
Dioleoyl or dipalmitoyl-sn-glycero-3-phosphoethanolamine-N-[4-(p-
maleimidomethyl)cyclohexane-carboxamide]
Dipalmitoyl-sn-glycero-3-phosphoethanolamine-N-[4-(p-
maleimidophenyl)butyramide]
Dipalmitoyl-sn-Glycero-3-Phosphothioethanol
Dipalmitoyl-sn-glycero-3-phosphoethanolamine-N-(succinyl)
Distearoyl-sn-glycero-3-phosphocholine (N-propynyl)
Dipalmitoyl-sn-glycero-3-phosphoethanolamine-N-dibenzocyclooctyl
Distearoyl-sn-glycero-3-phosphocholine (N-azidoethyl)
Dimyristoyl-sn-glycero-3-phosphoethanolamine-N-[methoxy(polyethylene
glycol)-350]
Dimyristoyl-sn-glycero-3-phosphoethanolamine-N-[methoxy(polyethylene
glycol)-550]
Dimyristoyl-sn-glycero-3-phosphoethanolamine-N-[methoxy(polyethylene
glycol)-750]
Dimyristoyl-sn-glycero-3-phosphoethanolamine-N-[methoxy(polyethylene
glycol)-1000]
Dimyristoyl-sn-glycero-3-phosphoethanolamine-N-[methoxy(polyethylene
glycol)-2000]
Distearoyl-sn-glycero-3-phosphoethanolamine-N-[carboxy(polyethylene
glycol)-2000]
Distearoyl-sn-glycero-3-phosphoethanolamine-N-[amino(polyethylene
glycol)-2000]
Distearoyl-sn-glycero-3-phosphoethanolamine-N-[succinyl(polyethylene
glycol)-2000]
Distearoyl-sn-glycero-3-phosphoethanolamine-N-[maleimide(polyethylene
glycol)-2000]
Distearoyl-sn-glycero-3-phosphoethanolamine-N-[biotinyl(polyethylene
glycol)-2000]
Distearoyl-sn-glycero-3-phosphoethanolamine-N-[carboxy(polyethylene
glycol)-2000, NHS ester]
Dipalmitoyl-sn-glycero-3-phosphoethanolamine-N-[azido(polyethylene
glycol)-2000]
Distearoyl-sn-glycero-3-phosphoethanolamine-N-[azido(polyethylene
glycol)-2000]
Dioleoyl-sn-glycero-3-phosphoethanolamine-N-[azido(polyethylene
glycol)-2000]
Dioleoyl-sn-glycero-3-phosphoethanolamine-N-[azido(polyethylene
glycol)-2000]
Dioleoyl-sn-glycero-3-phosphoethanolamine-N-[amino(polyethylene
glycol)-2000]
Dioleoyl-sn-glycero-3-phosphoethanolamine-N-[carboxy(polyethylene
glycol)-2000]
Dimyristoyl-sn-glycero-3-phosphoethanolamine-N-[methoxy(polyethylene
glycol)-3000]
Dimyristoyl-sn-glycero-3-phosphoethanolamine-N-[methoxy(polyethylene
glycol)-5000]
Distearoyl-sn-glycero-3-phosphoethanolamine-N-[amino(polyethylene
glycol)-5000]
16:0 Azidoethyl sphingomelin
1,2-distearoyl-sn-glycero-3-phosphocholine (N-azidoethyl)
(2S,3R,E)-2-amino-14-azidotetradec-4-ene-1,3-diol
1,2-distearoyl-sn-glycero-3-phosphoethanolamine-N- [azido
(polyethylene glycol)-2000]

### Pharmaceutical Formulations.

In various embodiments pharmaceutical formulations contemplated herein contain synthetic exosomes as described herein and a pharmaceutically acceptable carrier. The term “carrier” typically refers to an inert substance used as a diluent or vehicle for the pharmaceutical formulation. The term can also encompass a typically inert substance that imparts cohesive qualities to the composition. Typically, the physiologically acceptable carriers are present in liquid form. Examples of liquid carriers include, but not limited to, physiological saline, phosphate buffer, normal buffered saline (135-150 mM NaCl), water, buffered water, 0.4% saline, 0.3% glycine, 0.3M sucrose (and other carbohydrates), glycoproteins to provide enhanced stability (e.g., albumin, lipoprotein, globulin, etc.) and the like. Since physiologically acceptable carriers are determined in part by the particular composition being administered as well as by the particular method used to administer the composition, there are a wide variety of suitable formulations of pharmaceutical compositions of the present invention (see, e.g., Remington's Pharmaceutical Sciences, Maak Publishing Company, Philadelphia, Pa., 17th ed. (1985)).

In various embodiments the pharmaceutical formulations can be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. In certain embodiments the compositions can contain pharmaceutically acceptable auxiliary substances as required to approximate physiological conditions, such as pH adjusting and buffering agents, tonicity adjusting agents, wetting agents and the like, e.g., sodium acetate, sodium lactate, sodium chloride, potassium chloride, calcium chloride, sorbitan monolaurate and triethanolamine oleate. Sugars can also be included for stabilizing the compositions, such as a stabilizer for lyophilized compositions.

Pharmaceutical compositions suitable for parenteral administration, such as, for example, by intraarticular, intravenous, intramuscular, intratumoral, intradermal, intraperitoneal and subcutaneous routes, can include aqueous and non-aqueous, isotonic sterile injection solutions. In certain embodiments the injection solutions can contain antioxidants, buffers, bacteriostats and solutes that render the formulation isotonic with the blood of the intended recipient, and aqueous and non-aqueous sterile suspensions that can include suspending agents, solubilizers, thickening agents, stabilizers and preservatives. Injection solutions and suspensions can also be prepared from sterile powders, such as lyophilized synthetic exosomes. In certain embodiments the compositions can be administered, for example, by intravenous infusion, intraperitoneally, intravesically or intrathecally. In various embodiments parenteral administration and intravenous administration are also contemplated. The formulations of liposome compositions can be presented in unit-dose or multi-dose sealed containers, such as ampoules and vials.

In certain embodiments the pharmaceutical compositions are formulated for systemic administration as an injectable.

In certain embodiments the pharmaceutical compositions are formulated for administration as an aerosol, e.g., for oral and/or nasal inhalation.

In certain embodiments the pharmaceutical compositions are formulated for topical deliver, intradermal delivery, subdermal delivery and/or transdermal delivery.

In certain embodiments the pharmaceutical compositions are formulate for application to oral mucosa, vaginal mucosa, and/or rectal mucosa.

In certain embodiments the pharmaceutical composition is in unit dosage form. In such form, the composition is subdivided into unit doses containing appropriate quantities of the active component (synthetic exosome). The unit dosage form can be a packaged composition, the package containing discrete quantities of the pharmaceutical composition. The composition can, if desired, also contain other compatible therapeutic agents.

In certain embodiments the synthetic exosomes described herein can be delivered through the skin using conventional transdermal drug delivery systems, i.e., transdermal “patches” wherein the synthetic exosomes or formulations thereof) are typically contained within a laminated structure that serves as a drug delivery device to be affixed to the skin. In such a structure, the synthetic exosomes and/or formulations thereof are typically contained in a layer, or “reservoir,” underlying an upper backing layer. It will be appreciated that the term “reservoir” in this context refers to a quantity of synthetic exosomes, and/or formulations thereof that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates polyurethanes, and the like. Alternatively, the synthetic exosome and/or synthetic exosomes formulation reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the synthetic exosomes and/or formulations thereof) and any other materials that are present.

Alternatively, other pharmaceutical delivery systems can be employed. For example, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

### Therapeutic Moieties Delivered Using Synthetic Exosomes.

The synthetic exosomes described herein can readily be used to transport any of a number of therapeutic moieties across the blood brain barrier and effectively deliver those therapeutic moieties to the central nervous system (e.g., to the brain). Illustrative therapeutic moieties include, but are not limited to a protein, an antibody, an enzyme, a DNA encoding an inhibitory RNA, an inhibitory RNA or a micoRNA (miRNA), and/or a small organic molecule. It will be recognized that in certain embodiments, a single therapeutic moiety is delivered using the synthetic exosomes described herein, while in other embodiments, a plurality of therapeutic moieties are delivered using the synthetic exosomes described herein. Thus, for example, in certain embodiments, 2, 3, 4, or more therapeutic moieties can be encapsulated in a single synthetic exosome.

Small Organic Molecules.

Illustrative small organic molecules include, but are not limited to hydantoins as described in PCT Publication No: WO 2014/127042 (PCT/US2014/016100), disulfiram and/or analogues thereof, honokiol and/or analogues thereof, tropisetron and/or analogues thereof, nimetazepam and/or analogues thereof (see, e.g., PCT/US2011/048472 (WO 2012/024616), tropinol-esters and/or related esters and/or analogues thereof (see, e.g., PCT/US2012/049223 (WO 2013/019901), TrkA kinase inhibitors (e.g., ADDN-1351) and/or analogues thereof (see, e.g., PCT/US2012/051426 (WO 2013/026021 A2), D2 receptor agonists and alphal-adrenergic receptor antagonists, and APP-specific BACE Inhibitors (ASBIs) as described in PCT/US2013/032481 (WO 2013/142370), including, but not limited to galangin, a galangin prodrug, rutin, a rutin prodrug, and other flavonoids and flavonoid prodrugs as described or claimed therein.

Non-limiting examples of additional therapeutic agents include drugs selected from the group consisting of: (a) drugs useful for the treatment of Alzheimer's disease and/or drugs useful for treating one or more symptoms of Alzheimer's disease, (b) drugs useful for inhibiting the synthesis Aβ, and (c) drugs useful for treating neurodegenerative diseases.

Other small organic molecules include various chemotherapeutic compounds including but not limited to mitoxantrone, a retinoic acid derivative, doxirubicin, vinblastine, vincristine, cyclophosphamide, ifosfamide, cisplatin, 5-fluorouracil, a camptothecin derivative, interferon, tamoxifen, and taxol, abraxane, doxorubicin, pamidronate disodium, anastrozole, exemestane, cyclophosphamide, epirubicin, toremifene, letrozole, trastuzumab, megestroltamoxifen, paclitaxel, docetaxel, capecitabine, goserelin acetate, zoledronic acid, and the like. .

SE-Mediated sAPPα (or Other Protein) Delivery to the Brain and CNS.

Delivery of sAPPα to the brain is believed to be clinically beneficial not only in Alzheimer's disease, Amyotrophic lateral sclerosis (ALS), cerebral amyloid angiopathy, Huntington's disease, but also for Traumatic brain injury (TBI), and Stroke therapy.

sAPPα is −100 kDa protein fragment produced by the normal processing of the amyloid precursor protein (APP) by α-secretase. Since sAPPα is a large protein and subject to proteolysis, we encapsulated sAPPα in deformable synthetic exosomes (SE-hsAPPα) to increase the likelihood of brain delivery.

SEs containing sAPPα (SE-hsAPPα) were synthesized using flow chemistry in a microfluidic reactor to assist in efficient and reproducible production of SEs. The SE-hsAPPα were tested in CHO-7W cells to confirm sAPPα release and inhibition of BACE1 as determined by decreases in BACE1 APP-derived cleavage product sAPPβ in cells (see, e.g., FIG. 3). SE-hsAPPα significantly decreased sAPPβ.

We also assessed the ability of SE-hsAPPα to penetrate the blood brain barrier (BBB) by measuring the sAPPα levels in the mouse brain after intravenous (IV) injection (FIG. 4). sAPPα in the brain was −13 nM at 1h after injection.

In view of the surprising discovery that encapsulation of sAPPα in SEs permits effective delivery of this large protein across the blood-brain barrier into the brain and the delivered sAPPα appears to retain its activity to allosterically inhibit BACE, it is believed the SEs (SE-hsAPPα) find utility as therapeutic and/or prophylactic agents in a number of contexts.

Specifically it is believed the SEs containing sAPPα comprising sAPPα find utility in the treatment or prevention of Alzheimer's disease and/or mild cognitive impairment (MCI) associated with amyloidogenic pathology. It is also believed that SEs containing sAPPα can be used prophylactically to slow or prevent the progression from an asymptomatic condition to MCI, and/or from an asymptomatic condition to pre-Alzheimer's disease or early stage Alzheimer's disease, and/or to slow or stop the progression of Alzheimer's disease.

It is also believed the synthetic exosomes containing sAPPα may be clinically beneficial not only in Alzheimer's disease, but in Amyotrophic lateral sclerosis (ALS), cerebral amyloid angiopathy, and also for Traumatic brain injury (TBI) and Stroke therapy. In Alzheimer's disease the levels of sAPPα are reduced in the brain especially in patients carrying a ApoE4 allele. sAPPα levels are also modulated in several CNS disorders including Amyotrophic lateral sclerosis (ALS) and Parkinson's disease (PD). In Traumatic brain injury and Stroke there is a short-term increase in Aβ levels in the brain and this increase can be modulated by short term delivery of the allosteric BACE inhibitor sAPPα.

Accordingly, in certain embodiments, synthetic exosomes (SEs) containing sAPPα are provided as well as pharmaceutical formulations comprising these SEs. Methods of prophylaxis and/or treatment using the SEs are also provided. Additionally, methods of making (fabricating) the SEs are provided. Other proteins including, but not limited to the IDUA gene product (alpha-L-iduronidase) for mucopolysaccharidosis type I (MPS I) or acid sphingomyelinase (ASM) (aka SMPD1) for Neimann Pick disease (NPD) are contemplated.

SE-Mediated Antibody Delivery to the Brain and CNS.

It was a surprising discovery that the synthetic exosomes (also known as SEs) described herein can be used to effectively facilitate passage of “SE encapsulated” antibodies across the blood brain barrier. Illustrative antibodies include, but are not limited to antibodies useful for the treatment of brain cancers and/or neurodegenerative diseases (e.g., Alzheimer’ disease, amyloid-related mild cognitive impairment (MCI), Huntington's disease (HD), amyotrophic lateral sclerosis (ALS), Parkinson's disease (PD), and the like).

Alzheimer's Disease

Alzheimer's disease (AD) is a progressive neurodegenerative disorder characterized clinically by memory and cognitive dysfunction. The disease is generally classified into two types: sporadic AD (SAD) and familial AD (FAD). Three genes lead to familial AD (FAD)—amyloid precursor protein (APP), presenilin 1 (PS1), and presenilin (PS2)—while the ε4 allele of the apolipoprotein E gene has been identified as the major risk factor for sporadic AD (SAD) The neuropathology of AD is characterized by two types of lesions, extracellular senile plaques and intracellular neurofibrillary tangles (NFTs), which are composed of, respectively, β-amyloid (Aβ), a cleavage product of APP, 4 and aberrantly phosphorylated tau, a microtubule-associated protein.

Research has established that most neurodegenerative diseases including Alzheimer's, Lewy Body and other dementias, Parkinson's and prion diseases develop and progress along similar paths. In each disease, a particular protein undergoes a change in its shape from a soluble, physiologically functional protein to a protein that has lost the ability to perform its required tasks in the brain, starting off a chain reaction of binding to each other with little control. These aggregates become toxic to brain cells.

It is believed that attacking an early form of these proteins when they change their shape could prevent their formation into aggregates that lead to plaques and tangles, or neutralize their capacity to spread throughout the brain, and stop the progression of a particular neurological disease.

In certain embodiments this can be accomplished by the use of antibodies (e.g., monoclonal antibodies) that react to an intermediate, or “oligomer” state of the amyloid and tau proteins seen in Alzheimer's disease, as well as to prion disease proteins.

Researchers have shown that a number of antibodies directed against proteins comprising amyloid deposits and/or proteins involved in the amyloidogenic processes can slow, halt, or reverse the formation of amyloid plaques and presumably the associated cog native decline.

Illustrative targets for the treatment of Alzheimer's disease include, but are not limited to Aβ, mutant Aβ, tau, mutant tau, apoE, and the like (see, e.g., Table 5).

TABLE 5
Illustrative, but non-limiting list of antibodies for the treatment
of Alzheimer's disease and their respective targets.

| Antibody | Target |
|---|---|
| AAB-003 | Aβ |
| Bapineuzumab | Aβ (epitope: aa 1-5 monomers, oligomers, |
| | fibrils, ARIA-E) |
| Ponezumab | Aβ (epitope: aa 13-24 monomers) |
| RG7345 | Tau |
| Solanezumab | Aβ (epitope aa 16-26 monomers) |
| GSK933776 | Aβ |
| JNJ-63733657 | Tau |
| BIIB076 | Tau |
| LY2599666 | Aβ |
| MEDI1314 | Aβ |
| SAR228810 | Aβ (protofibrils, and low molecular weight |
| | amyloid-β) |
| BAN2401 | Aβ protofibrils |
| BIIB092 | Tau |
| C2B8E12 | Tau |
| LY3002813 | Aβ (Aβ(p3-42), a pyroglutamate form of Aβ |
| LY3303560 | Tau |
| RO 7105705 | Tau |
| Aducanumab | Aβ (epitope: 3-6, fibrils) |
| Crenezumab | Aβ |
| Gantenerumab | Aβ (epitope: aa 3-12, 18-27 oliogmers, |
| | fibrils ARIA-E) |
| HAE-4 | apoE |
| 9D5 | Pyroglutamate-3 Aβ |
| BIIB037/BART | Insoluble fibrillar human amyloid-β |

In certain embodiments the antibodies target mutant A(3s which include, but are not limited to APPsw, APP A713T, pyroglutamate-3 A13, and the like.

Without being bound to a particular theory, it is believed the synthetic exosomes described herein can effectively deliver these and other antibodies across the blood brain barrier permitting effective doses to act on the brain.

Amyotrophic Lateral Sclerosis (ALS)

Antibodies directed against the HERV-K envelope protein or SOD1 are believed to be effective candidates for the treatment of amyotrophic lateral sclerosis (ALS). Accordingly, in certain embodiments, synthetic exosomes containing anti-HEV-K or anti-SOD1 antibodies are contemplated.

Huntington's Disease.

The antibody VX15 is a monoclonal antibody that blocks the activity of semaphorin 4D (SEMA4D), a molecule that is believed to promote chronic inflammatory responses in the brain, and is believed to be effective in the treatment of Huntington's disease (HD). VX15 is the Company's novel clinical stage monoclonal antibody that blocks the activity of semaphorin 4D (SEMA4D), a molecule that is believed to promote chronic inflammatory responses in the brain. Accordingly, in certain embodiments, synthetic exosomes containing VX15 or other anti-SEMA4D antibodies are contemplated.

Parkinson's Disease.

The monoclonal antibody PRX002 (prasinezumab), targets α-synuclein and is believed to inhibit cell-to-cell transmission of alpha-synuclein and modify disease progression in Parkinson's disease (PD). Accordingly, in certain embodiments, synthetic exosomes containing prasinezumab or other anti-α-synuclein antibodies are contemplated.

Brain Cancers.

The synthetic exosomes described herein can also be used to transport antibodies (or chemotherapeutic drugs) useful for the treatment of brain tumors (CNS-tumors) across the blood brain barrier. Cancer cells sometimes find ways to use checkpoints (molecules on certain immune cells that need to be activated (or inactivated) to start an immune response) to avoid being attacked by the immune system. Drugs (e.g., antibodies) that target these checkpoints can provide effective therapies for the treatment of cancers.

PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it basically tells the T cell to leave the other cell alone. Some cancer cells have large amounts of PD-L1, which helps them evade immune attack. Monoclonal antibodies that target either PD-1 or PD-L1 can block this binding and boost the immune response against cancer cells. Illustrative PD-1 inhibitors include, but are not limited to Pembrolizumab (KEYTRUDA®), Nivolumab (OPDIVO®), Cemiplimab (LIBTAYO®), and the like.

Other checkpoint inhibitors include, but are not limited to PD-L1 inhibitors. Illustrative PD-L1 inhibitors include, but are not limited to Atezolizumab (TECENTRIQ®), Avelumab (BAVENCIO®), Durvalumab (IMFINZI®), and the like.

CTLA-4 is another protein on some T cells that acts as a type of “off switch” to keep the immune system in check. Ipilimumab (YERVOY®) is a monoclonal antibody that attaches to CTLA-4 and stops it from working. This can boost the body's immune response against cancer cells.

Other antibodies useful in the treatment of cancer include, but are not limited to anti-CD52 antibodies, anti-CD47 antibodies, anti-VEGF antibodies (e.g., bevacizumab), anti-CD20 (e.g., rituximab), anti-HER2 (e.g., trastuzumab), and the like. Without being bound to a particular theory, it is believed that SEs containing anti-cancer antibodies can be effective in treating brain cancers including, but not limited to Acoustic Neuroma, Astrocytoma (e.g., Grade I—Pilocytic Astrocytoma, Grade II—Low-grade Astrocytoma, Grade III—Anaplastic Astrocytoma,Grade IV—Glioblastoma (GBM)), Chordoma, CNS Lymphoma, Craniopharyngioma, Other Gliomas) including, but not limited to Brain Stem Glioma, Ependymoma, Mixed Glioma, Optic Nerve Glioma, Subependymoma), Medulloblastoma, Meningioma, Metastatic Brain Tumors, Oligodendroglioma, Pituitary Tumors, Primitive Neuroectodermal (PNET), Schwannoma

The foregoing antibodies are illustrative and non-limiting. Using the teachings provided herein the synthetic exosomes can readily be used to deliver any of a wide number of antibodies across the blood brain barrier (BBB).

SE-Mediated Delivery of Missing Enzymes

In certain embodiments we will use the SE technology to deliver missing enzymes to the brain in rare diseases such as but not limited to: beta-N-acetylhexosaminidase A in Tay Sachs disease, phenylalanine hydroxylase in PKU, Iduronidase (IDUA) in MPS-1, iduronate-2-sulfatase (IDS) in MPS-II, heparan N-sulfatase or alpha-N-acetylglucosaminidase or heparan-alpha-glucosaminide N-acetyltransferase or N-acetylglucosamine 6-sulfatase in MPS-III, N-acetyl-galactosamine-6-sulfatase or beta-galactosidase in MPS-IV, Acidic sphingomyelinase (ASM1) for Niemann Pick's disease, aminoacylase 2 for Canavan's disease, Carnitine palmitoyltransferase II, Kynurenine-3-monooxygenase.

SE-Mediated ASO Delivery to the Brain and CNS.

In certain embodiments, SE's can be used for delivery of antisense oligo nucleotides (ASO) for CNS disorders including but not limited to: spinal muscular atrophy (SMA), ALS, Huntington's disease, Parkinson's disease and Alzheimer's disease.

SE-Mediated CRISPR/Cas9 Delivery to the Brain and CNS.

In certain embodiments the synthetic exosomes described herein can contain the components of a CRISPR/cas system and effectively deliver these components across the blood brain barrier. pathogenic mutations in monogenic diseases including AD, PD, ALS, FXS, SCA and SBMA. Such packaged CRISPR/cas components can involve providing the appropriate guide RNA (sgRNA) and the Cas enzyme in the synthetic exosomes and administering the synthetic exosomes, e.g., via intravenous infusion.

In certain embodiments the components of the CRISPR/cas system include a nucleic acid that encodes and expresses a CRISPR endonuclease and a nucleic acid that is, or that encodes, a desired guide RNA. In certain embodiments the SE packaged nucleic acid encodes both a CRISPR endonuclease and a guide RNA. In certain embodiments the components of the CRISPR/cas system comprise a CRISPR endonuclease protein and a nucleic acid that is, or that encodes a guide RNA.

Without being bound to a particular theory, it is believed that the synthetic exosome-packaged CRISPR/cas system can be used in vivo for the correction of pathogenic mutations in, e.g., monogenic diseases including, but not limited to Alzheimer's disease (AD), Parkinson's disease (PD), Huntington's disease (HD), amyotrophic lateral sclerosis (ALS), fragile X syndrome (FXS), autosomal dominant spinocereberal ataxis (SCAs) and spinal bulbar muscular atrophy (SBMA), and correction of autosomal recessive genetic disorder that is caused by a deficiency in the expression or function of the Ataxia Telangiectasia Mutated (ATM) protein, and the like.

SE Delivery of CRISPR/Cas for Alzheimer's Disease.

Because the majority of AD cases are sporadic with the trigger unknown, the use of CRISPR/Cas9 would not seem a viable treatment approach. This is supported by the fact that a very small percentage of cases (<1%) are caused by known mutations in the APP protein or genes products involved in processing APP to form beta-amyloid. What is certain is that, although these mutations make up a small percentage of known AD cases, they all lead to enhanced production of the beta-amyloid peptide (Bettens et al. (2013) Lancet. Neurol. 12:92-104). Other known mutations that lead to early-onset AD include those to presenilin 1 (PSEN1) and presenilin 2 (PSEN2) (Schellenberg et al. (1992) Science, 258: 668-671; Levy-Lahad et al. (1995) Science, 269: 970-973). The net effect of mutations to these two genes is enhanced production of beta-amyloid (1-42) perhaps by shifting the cleavage site in APP (Vetrivel et al. (2006) Mol. Neurodegener. 1:4).

Clearly, the potential for CRISPR/cas9 in potentially correcting these autosomal-dominant mutations is real. This is supported by studies that have analyzed the potential of correcting similar kinds of mutations using this gene editing system. For example, CRISPR/Cas9 was used to correct a presenilin (PSEN2) autosomal dominant mutation in iPSC-derived neurons. In this study, the authors generated basal forebrain cholinergic iPSC neurons from an individual carrying the PSEN2N141I mutation (Ortiz-Virumbrales et al. (2017) Acta Neuropathol. Commun. 5: 77). In this study, CRISPR/Cas9 corrected the N141I mutation demonstrated by Sanger sequencing that led to a normalization of the Aβ 42/40 ratio. Functionally, the CRISPR/Cas9 correction of the PSEN2 mutation reversed electrophysiological deficits. This study was supported by previous studies that have also used CRISPR/Cas9 to correct familial AD mutations in the PSEN gene in patient-derived iPSCs (Pires et al. (2016) Stem Cell Res. 17: 285-288; Poon et al. (2016) Stem Cell Res. 17: 466-469).

CRISPR/cas9 has also been used to knock out the Swedish APP mutation in patient-derived fibroblasts leading to a 60% reduction in secreted beta-amyloid (Gyorgy et al. (2018) Mol. Ther. Nucleic Acids, 11: 429-440).

Additionally, sgRNAs targeting the extreme C-terminus of APP led to robust APP-editing (see, e.g., A CRISPR/Cas9 based strategy to manipulate the Alzheimer's amyloid pathway (bioRxiv preprint doi: //doi.org/10.1101/310193) which is incorporated herein by reference for the CRISPR/cas components, e.g., sgRNA, described therein). FIG. 1a in this reference illustrates the protospacer adjacent motif—PAM—site and genomic target recognized by the sgRNA. The sgRNA appeared to have reciprocal effects on sAPPα and APPβ cleavage providing confidence that the gene editing strategy favorably manipulated the amyloid pathway.

Other targets for CRISPR to treat AD include, but are not limited to tau and BACE1. Tau plays a critical role not only in AD pathophysiology but also in various other neurodegenerative disorders, including, but not limited to FTLD, which is also called frontotemporal dementia (Spillantini & Goedert M (1998) Trends Neurosci. 21: 428-433; Goedert et al. (1998) Neuron, 21: 955-958; Grundke-Iqbal et al. (1986) J. Biol. Chem. 261: 6084-6089; Grundke-Iqbal et al. (1986) Proc. Natl. Acad. Sci. USA, 83: 4913-4917; Ittner et al. (2010) Cell, 142: 387-397). In order to successfully treat, AD it can be desirable to simultaneously target both Aβ as well as tau. It is believed CRISPR and readily used to eliminate toxic tau forms by gene silencing.

Similarly, the gene Bacel, which encodes β-secretase 1 is required for the production of amyloid-β (Aβ) peptides and, therefore, has a central role in the accumulation of Aβ that occurs in AD. This gene can be targeted using a CRISPR/cas system (see, e.g., Park, et al. (2019) Nat. Neurosci. 22: 524-528.

Similarly, the risk factor for AD is ApoE4, can be modified by CRISPR to ApoE3 or ApoE2.

These and other targets suitable for the treatment of Alzheimer's disease or FTLD are readily identified by those of skill in the art and the appropriate CRISPR/cas system components can readily be delivered to the central nervous system (CNS) using the synthetic exosomes described herein.

SE Delivery of CRISPR/Cas for Parkinson's Disease.

Cells and animals overexpressing α-synuclein have proven to be a useful model for Parkinson's disease. IN a new study, Cas9′ cutting ability was deactivated and the protein was engineered so that after binding to a target site it recruits transcription factors. A guide RNA strand has been identified that has a powerful effect in keeping cells alive much more effectively than any of the individual genes that have been previously found to protect the cells used in the study (see, e.g., Chen et al. (2017) Mol. Cell, 68(1): 247-257 which is incorporated herein by reference for the CRISPR/cas system components (including guide RNA) described therein.

Further genetic screening revealed that many of the genes turned on by this guide RNA strand are chaperone proteins, which help other proteins fold into the correct shape. The researchers hypothesize that these chaperone proteins may assist in the proper folding of alpha-synuclein, which could prevent it from forming clumps.

These and other CRISPR/cas system components for the treatment of Parkinson's disease or other pathologies characterized by α-synuclein aggregation can readily be delivered to the central nervous system (CNS) using the synthetic exosomes described herein.

SE Delivery of CRISPR/Cas for Huntington Disease.

Huntington disease (HD). HD is caused by the expansion of CAG repeats in exon 1 of the HTT gene, which encodes for mutant huntingtin (mHTT), a large protein consisting of large polyglutamine repeats in the N-terminal domain of mHTT that most likely exhibits a toxic-gain of function. One potential strategy to treat HD could be using CRISPR/Cas9 to selectively suppress the expression of mHTT. The rationale for this approach comes from a previous study demonstrating that the application of RNA interference improved motor and neuropathological abnormalities in a HD mouse model. Since this study, the CRISPR/Cas9 gene editing system has been successfully applied to HD (Shin et al. (2016) Hum. Mol. Genet. 25: 4566-4576; Kolli et al. (2017) Int. J. Mol. Sci. 18(4): 754; Monteys et al. (2017)Mol. Ther. 25: 12-23).

Recently, CRISPR/Cas9 was used to selectively suppress the entire HTT and mHTT gene in an in vivo mouse model of HD (non-allele specific approach) (Yang et al. (2017) J. Clin. Invest. 127: 2719-2724). By way of illustration the guide RNAs used in this study (HTT-gRNAs T1, T2, T3, and T4) were designed to target the regions flanking the polyQ region and are incorporated herein by reference for their sequence information.

These and other CRISPR/cas system components for the treatment of Huntington disease can readily be delivered to the central nervous system (CNS) using the synthetic exosomes described herein.

SE Delivery of CRISPR/Cas for Amyotrophic Lateral Sclerosis.

Amyotrophic lateral sclerosis (ALS) is a fatal and incurable neurodegenerative disease characterized by the progressive loss of motor neurons in the spinal cord and brain. In particular, autosomal dominant mutations in the superoxide dismutase 1 (SOD1) gene are responsible for ˜20% of all familial ALS cases. The clustered regularly interspaced short palindromic repeats (CRISPR)—CRISPR-associated (Cas9) genome editing system holds the potential to treat autosomal dominant disorders by facilitating the introduction of frameshift-induced mutations that can disable mutant gene function.

It has been demonstrated that CRISPR-Cas9 can be harnessed to disrupt mutant SOD1 expression. Such genome editing can significantly reduce or eliminate mutant SOD1 protein resulting in improved motor function and reduced muscle atrophy (e.g., Gaj et al. (2017) Sci. Adv., 3: eaar3952).

These and other CRISPR/cas system components for the treatment of ALS can readily be delivered to the central nervous system (CNS) using the synthetic exosomes described herein.

SE Delivery of CRISPR/Cas for Fragile X Syndrome.

Fragile X syndrome (FXS) is a common cause of intellectual disability that is most often due to a CGG-repeat expansion mutation in the FMR1 gene that triggers epigenetic gene silencing. Epigenetic modifying drugs can only transiently and modestly induce FMR1 reactivation in the presence of the elongated CGG repeat. As a proof-of-principle, the expanded CGG-repeat was excised in both somatic cell hybrids containing the human fragile X chromosome and human FXS iPS cells using the CRISPR/Cas9 genome editing (see, e.g. Xie et al. (2016) PLOS ONE 11(10): e0165499). Transcriptional reactivation in approximately 67% of the CRISPR cut hybrid colonies and in 20% of isolated human FXS iPSC colonies. The reactivated cells produced FMRP and exhibited a decline in DNA methylation at the FMR1 locus. These data demonstrate the excision of the expanded CGG-repeat from the fragile X chromosome can result in FMR1 reactivation.

These and other CRISPR/cas system components for the treatment of fragile X syndrome can readily be delivered to the central nervous system (CNS) using the synthetic exosomes described herein.

SE Delivery of CRISPR/Cas for Autosomal Dominant Spinocereberal Ataxis (SCAs) and Spinal Bulbar Muscular Atrophy (SBMA).

Repeat expansion disorders are a class of genetic diseases that are caused by expansions in DNA repeats. The DNA repeats come in various sizes from single nucleotides to dodecamers or longer. The threshold at which the repeat expansions become symptomatic varies with the specific disease. There are over 40 distinct diseases now known to be caused by these expansions in DNA sequence. Remarkable progress during the last three decades has defined causative mutations that drive pathogenesis in a large number of these diseases. Expansion of CAG, GCG, CTG, CGG, and CAAA repeats both in coding and non-coding sequences in distinct genes results in a diverse group of diseases with mechanisms linked to protein levels or toxicity, RNA, and/or both,

Spinal and Bulbar Muscular Atrophy/Kennedy's Disease

The cause of Kennedy's disease has been shown to be a CAG expansion in the androgen receptor (AR) gene (La Spada et al. (1991) Nature, 352(6330): 77-79). Spinal and bulbar muscular atrophy (SBMA) is a slow progressive neuromuscular disorder in which the lower motor neurons and muscles degenerate. SBMA is X-linked and therefore mainly affecting males, with some exceptions discussed in the Arnold and Merry review (Arnold & Merry Molecular Mechanisms and Therapeutics for SBMA/Kennedy's Disease. Neurotherapeutics. 2019). The symptoms include gynecomastia, testicular atrophy, and reduced fertility, all which correlate with the known function of AR as a transcription factor binding androgen hormones. AR is involved in the male reproductive and skeletal system and female fertility. Given the known function of AR, the normal lifespan of SBMA patients, and the rapid progress in the field, it is surprising that after almost thirty years of investigation, we do not have a therapeutic treatment for this disease. Merry reviews the complexity of the underlying pathogenesis of this disorder and highlights steps that may lead to therapeutics for SBMA/Kennedy's disease (Id.).

SCA1

The genetic cause of Spinocerebellar ataxia type 1 (SCA1) was reported in 1993 (Orr et al. (1993) Nat. Genet. 4(3): 221-226; Srinivasan & Shakkottai (2019) Neurotherapeutics, DOI:10.1007/s13311-019-00763-y). SCA1 is an autosomal-dominant disorder characterized by neurodegeneration of the cerebellum, spinal cord, and brainstem and has a polyQ expansion in the ataxin-1 protein.

SCA3

Spinocerebellar ataxia type 3 (SCA3), also known as Machado-Joseph disease (MJD), is caused by a polyQ expansion in the ataxin-3 protein (Takiyama et al. (1993) Nat. Genet. 4(3): 300-304). Ataxin-3 is a ubiquitin ligase and Da Silva et al. present a unifying molecular mechanism for disease pathogenesis focusing on the disruption of protein homeostasis (Da Silva et al. (2019) Neurotherapeutics, 16(4): 1009-1031). This molecular mechanism is common to all polyQ diseases and key networks are likely shared between the diseases.

SCA2

In 1996, the CAG expansion in ataxin-2 gene was discovered to cause Spinocerebellar ataxia type 2 (SCA2). Egorova and Bezprozvanny describe the epidemiology, the function of ataxin-2 in RNA metabolism and stress granules, the molecular changes underlying Purkinje cell loss, the cerebellar-thalamic cortical circuit dysregulation and ataxia, and molecular mechanisms that may be therapeutically targeted using, e.g., CRISPR/cas systems (Egorova & Bezprozvanny et al. (2019) 16(4): 1050-1073).

SCA7

Spinocerebellar ataxia type 7 (SCA7) is caused by a CAG expansion in the ataxin-7 gene and is characterized by neuronal loss in the cerebellum, brainstem, and retina. The major symptoms are cerebellar ataxia and blindness. The ataxin-7 protein is a subunit of the multiprotein SAGA complex which is involved in chromatin remodeling and there is now a detailed molecular understanding of how the polyQ expansion in ataxin-7 disrupts neuronal function (Niewiadomska-Cimicka & Trottier (2019) Neurotherapeutics, 16(4):1074-1096).

SCA17

SCA17 is caused by a CAG/CAA repeat expansion in the gene encoding the TATA box-binding protein (TBP) (Koide et al. (1999) Hum. Mol. Genet. 8(11): 2047-2053). TBP is a well-characterized transcription factor. Liu et al. discuss the use of CRISPR-Cas9 for the treatment of SCA17 (Liu et al. (2019) Neurotherapeutics, 6(4):1097-1105).

SCA31

SCA31 is an adult onset neurological disorder with progression cerebellar ataxia caused by degenerating Purkinje cell in the Japanese population. The genetics of SCA31 was elucidated in 2009 (Sato et al. (2009) Am. J. Hum. Genet. 85(5): 544-557). There is 2.5-3.8 kb insertion of the penta-nucleotide repeat (TGGAA)n stretch in the introns of thymidine kinase 2 (TK) and BEAN (brain expressed, associated with Nedd4). An emerging mechanism of RNA toxicity is apparent for this disease (Ishikawa & Nagai (2019) Neurotherapeutics, 6(4):1106-1114).

C9orf72 ALS/FTD

A hexanucleotide repeat expansion in the first intron of the C9orf72 gene has been identified as a case of amyotrophic lateral sclerosis (ALS) and frontotemporal dementia (FTD). Given its relatively recent discovery, there has been rapid progress in identifying key pathogenic events such as the gain of toxicity from bidirectional transcribed repeat-containing RNAs, nucleocytoplasmic transport defects common to a number of the expansion diseases, and how the expansion causes two different diseases. These advances have led to phase I clinical trials using antisense oligonucleotide therapies and it is believed that CRISPR/cas approaches can be even more effective.

The use of CRISPR-Cas9 for the treatment of the above-described diseases offers a promising area of neurotherapeutics.

In view of this, these and other CRISPR/cas system components for the treatment of the above-identified pathologies, can readily be delivered to the central nervous system (CNS) using the synthetic exosomes described herein.

SE Delivery of CRISPR/Cas for Ataxia Telangiectasia (A-T)

Ataxia telangiectasia (A-T) is a disease characterized by cerebellar wasting, or atrophy, and eventually lymphoma; caused by mutations in the ataxia telangiectasia mutated gene, or ATM. The mutations that cause disease are typically located in the kinase domain or in proximity to regulatory regions of the protein.

Mutations in the ATM gene affect the production or the activity of the ATM protein, leading to the symptoms associated with A-T. Fixing the faulty gene permanently will give the cells a chance to produce the correct protein. In the last few years, CRISPR has revolutionized the field of genome editing and shown remarkable success in progressing treatments for many genetic diseases that also rely on the correction of faulty genes.

CRISPR/cas system components for the treatment of Ataxia telangiectasia, can readily be delivered to the central nervous system (CNS) using the synthetic exosomes described herein.

The CRISPR/Cas System—Class 2 CRISPR/Cas Endonucleases

As noted above, the synthetic exosomes described herein are particularly well suited for the in vivo delivery of components of a CRISPR/cas system.

Compelling evidence has recently emerged for the existence of an RNA-mediated genome defense pathway in archaea and many bacteria that has been hypothesized to parallel the eukaryotic RNAi pathway (for reviews, see Godde and Bickerton (2006) J. Mol. Evol. 62: 718-729; Lillestol et al. (2006) Archaea 2: 59-72; Makarova et al. (2006) Biol. Direct 1: 7; Sorek et al. (2008) Nat. Rev. Microbiol. 6: 181-186). Known as the CRISPR-Cas system or prokaryotic RNAi (pRNAi), the pathway is believed to arise from two evolutionarily and often physically linked gene loci: the CRISPR (clustered regularly interspaced short palindromic repeats) locus, that encodes RNA components of the system, and the cas (CRISPR-associated) locus, that encodes proteins (see, e.g., Jansen et al. (2002) Mol. Microbiol. 43: 1565-1575; Makarova et al., (2002) Nucl. Acids Res. 30: 482-496; Makarova et al. (2006) Biol. Direct 1: 7; Haft et al. (2005) PLoS Comput. Biol. 1: e60). CRISPR loci in microbial hosts contain a combination of CRISPR-associated (Cas) genes as well as non-coding RNA elements capable of programming the specificity of the CRISPR-mediated nucleic acid cleavage. The individual Cas proteins do not share significant sequence similarity with protein components of the eukaryotic RNAi machinery, but have analogous predicted functions (e.g., RNA binding, nuclease, helicase, etc.) (see, e.g., Makarova et al. (2006) Biol. Direct 1: 7). The CRISPR-associated (cas) genes are often associated with CRISPR repeat-spacer arrays. More than forty different Cas protein families have been described. Of these protein families, Cast_appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

In class 2 CRISPR systems, the functions of the effector complex (e.g., the cleavage of target DNA) can be carried out by a single endonuclease (see, e.g., Zetsche et al. (2015) Cell, 163(3): 759-771; Makarova et al. (2015) Nat. Rev. Microbiol. 13(11): 722-736; Shmakov et al. (2015) Mol. Cell. 60(3): 385-397; and the like). As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9), type V CRISPR/Cas proteins (e.g., Cpf1, C2c1, C2C3), and type VI CRISPR/Cas proteins (e.g., C2c2). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex (e.g., and cleaving target DNA).

Type II CRISPR/Cas Endonucleases (e.g., Cas9)

In natural Type II CRISPR/Cas systems, Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual-(dgRNA) or single guide RNA (sgRNA) form a ribonucleoprotein (RNP) complex that can be targeted to a desired DNA sequence. Guided by a dual-RNA complex or a chimeric single-guide RNA, Cas9 generates site-specific DSBs or SSBs within double-stranded DNA (dsDNA) target nucleic acids, that are repaired either by non-homologous end joining (NHEJ) or homology-directed recombination (HDR).

In some embodiments a nucleic acid encoding a CRISPR endonuclease, or the endonuclease protein and a guide RNA or a nucleic guide RNA are provided as cargo(s) in the synthetic exosomes described herein.

A Cas9 protein forms a complex with a Cas9 guide RNA. The guide RNA provides target specificity to a Cas9-guide RNA complex by having a nucleotide sequence (a guide sequence) that is complementary to a sequence (the target site) of a target nucleic acid (as described elsewhere herein). The Cas9 protein of the complex provides the site-specific activity. In other words, the Cas9 protein is guided to a target site (e.g., stabilized at a target site) within a target nucleic acid sequence (e.g. genomic DNA) by virtue of its association with the protein-binding segment of the Cas9 guide RNA.

In some cases, the CRISPR/Cas endonuclease (e.g., Cas9 protein) is a naturally-occurring protein (e.g., naturally occurs in bacterial and/or archaeal cells). In other cases, the CRISPR/Cas endonuclease (e.g., Cas9 protein) is not a naturally-occurring polypeptide (e.g., the CRISPR/Cas endonuclease is a variant CRISPR/Cas endonuclease, a chimeric protein, and the like, e.g., in some cases the CRISPR/Cas endonuclease includes one or more NLSs).

Examples of suitable Cas9 proteins include, but are not limited to, those set forth in SEQ ID NOs:5-816 of PCT Application No: PCT/US2017/017255 (WO 2017/139505), which are incorporated herein by reference for the sequences described therein. Naturally occurring Cas9 proteins bind a Cas9 guide RNA, are thereby directed to a specific sequence within a target nucleic acid (a target site), and cleave the target nucleic acid (e.g., cleave dsDNA to generate a double strand break). A chimeric Cas9 protein is a fusion protein comprising a Cas9 polypeptide that is fused to a heterologous protein (referred to as a fusion partner), where the heterologous protein provides an activity (e.g., one that is not provided by the Cas9 protein). The fusion partner can provide an activity, e.g., enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc.). In some cases, a portion of the Cas9 protein (e.g., the RuvC domain and/or the HNH domain) exhibits reduced nuclease activity relative to the corresponding portion of a wild type Cas9 protein (e.g., in some cases the Cas9 protein is a nickase). In some cases, the Cas9 protein is enzymatically inactive, or has reduced enzymatic activity relative to a wild-type Cas9 protein (e.g., relative to Streptococcus pyogenes Cas9). In some cases, the Cas9 protein is enzymatically enhanced, e.g., or has enhanced enzymatic activity and/or specificity relative to a wild-type Cas9 protein (e.g., relative to Streptococcus pyogenes Cas9).

Assays to determine whether given protein interacts with a Cas9 guide RNA can be any convenient binding assay that tests for binding between a protein and a nucleic acid. Suitable binding assays (e.g., gel shift assays) will be known to one of ordinary skill in the art (e.g., assays that include adding a Cas9 guide RNA and a protein to a target nucleic acid).

Assays to determine whether a protein has an activity (e.g., to determine if the protein has nuclease activity that cleaves a target nucleic acid and/or some heterologous activity) can be any convenient assay (e.g., any convenient nucleic acid cleavage assay that tests for nucleic acid cleavage). Suitable assays (e.g., cleavage assays) will be known to one of ordinary skill in the art and can include adding a Cas9 guide RNA and a protein to a target nucleic acid.

In some cases, a chimeric Cas9 protein includes a heterologous polypeptide that has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In some cases, a chimeric Cas9 protein includes a heterologous polypeptide that has enzymatic activity that modifies a polypeptide (e.g., a histone) associated with target nucleic acid (e.g., methyltransferase activity, demethylase activity, acetyltransferase activity, deacetylase activity, kinase activity, phosphatase activity, u biquitin ligase activity, deu biquitinating activity, adenylation activity, deadenylation activity, SUMOylating activity, deSUMOylating activity, ribosylation activity, deribosylation activity, myristoylation activity or demyristoylation activity).

In some cases, a CRISPR/Cas endonuclease (e.g., a Cas9 protein) includes a heterologous polypeptide that provides for localization within the cell. For example, in some cases, a subject CRISPR/Cas endonuclease (e.g., a Cas9 protein) includes one or more (e.g., 2 or more, 3 or more, 4 or more, 5 or more, etc.) nuclear localization sequences (NLSs). The one or more (e.g., 2 or more, 3 or more, 4 or more, 5 or more, etc.) NLSs can be at any convenient position within the CRISPR/Cas endonuclease (e.g., a Cas9 protein), e.g., N-terminus, C-terminus, internal, etc. In some cases, a CRISPR/Cas endonuclease (e.g., a Cas9 protein) includes one or more (e.g., 2 or more, 3 or more, 4 or more, 5 or more, etc.) NLSs at the N-terminus and one or more (e.g., 2 or more, 3 or more, 4 or more, 5 or more, etc.) NLSs at the C-terminus.

Many Cas9 orthologs from a wide variety of species have been identified and in some cases the proteins share only a few identical amino acids. Identified Cas9 orthologs have similar domain architecture with a central HNH endonuclease domain and a split RuvC/RNaseH domain (e.g., RuvCI, RuvCII, and RuvCIII) (e.g., see Table 6). For example, a Cas9 protein can have 3 different regions (sometimes referred to as RuvC-I, RuvC-11, and RucC-III), that are not contiguous with respect to the primary amino acid sequence of the Cas9 protein, but fold together to form a RuvC domain once the protein is produced and folds. Thus, Cas9 proteins can be said to share at least 4 key motifs with a conserved architecture. Motifs 1, 2, and 4 are RuvC like motifs while motif 3 is an HNH-motif. The motifs set forth in Table 6 may not represent the entire RuvC-like and/or HNH domains as accepted in the art, but Table 6 does present motifs that can be used to help determine whether a given protein is a Cas9 protein.

TABLE 6
Four motifs that are present in Cas9 sequences from various species.
The amino acids listed in Table 1 are from the Cas9 from S. pyogenes
(SEQ ID NO: 26, see also SEQ ID NO: 5 in PCT/US2017/017255).

| Motif # | Motif | Amino acids (residue #s) | Highly conserved |
|---|---|---|---|
| 1 | RuvC-like I | IGLDIGTNSVGWAVI (7-21) | D10, G12, G17 |
| | | (SEQ ID NO: 1) | |
| 2 | RuvC-like II | IVIEMARE (757-766) | E762 |
| | | (SEQ ID NO: 2) | |
| 3 | HNH-motif | DVDHIVPQSFLKDDSIDNKVLTRSDKN | H840, N854, N863 |
| | | (887-863) (SEQ ID NO: 3) | |
| 4 | RuvC-like | HHAHDAYL (982-989) | H982, H983, |
| | III | (SEQ ID NO: 4) | A984, D986, A987 |

In some cases, a suitable Cas9 protein comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 as set forth in SEQ ID NOs:1-4, respectively (e.g., see Table 6), or to the corresponding portions in any of the amino acid sequences set forth in SEQ ID NOs:5-816 in PCT/US2017/017255).

In other words, in some cases, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 of the Cas9 amino acid sequence set forth in SEQ ID NO:26 (see also SEQ ID NO:5 in PCT/US2017/017255) (e.g., the sequences set forth in SEQ ID NOs:1-4, e.g., see Table 6), or to the corresponding portions in any of the amino acid sequences set forth in SEQ ID NOs 6-816 in PCT/US2017/017255.

In some cases, a suitable Cas9 protein comprises an amino acid sequence having 4 motifs, each of motifs 1-4 having 60% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 70% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 75% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 80% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 85% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 90% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 95% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 99% or more amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. In some cases, a suitable Cas9 protein comprises an amino acid sequence having 4 motifs, each of motifs 1-4 having 100% amino acid sequence identity to motifs 1-4 of the Cas9 amino acid sequence set forth as SEQ ID NO:26 (the motifs are in Table 6, and are set forth as SEQ ID NOs:1-4, respectively), or to the corresponding portions in any of the amino acid sequences set forth in SEQ ID NOs:6-816 in PCT/US2017/017255. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255.

In some cases, a suitable Cas9 protein comprises an amino acid sequence having 60% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255. In some cases, a suitable Cas9 protein comprises an amino acid sequence having 70% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255. In some cases, a suitable Cas9 protein comprises an amino acid sequence having 75% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816. In some cases, a suitable Cas9 protein comprises an amino acid sequence having 80% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255. In some cases, a suitable Cas9 protein comprises an amino acid sequence having 85% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255. In some cases, a suitable Cas9 protein comprises an amino acid sequence having 90% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255. In some cases, a suitable Cas9 protein comprises an amino acid sequence having 95% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816. In some cases, a suitable Cas9 protein comprises an amino acid sequence having 99% or more amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816. In some cases, a suitable Cas9 protein comprises an amino acid sequence having 100% amino acid sequence identity to amino acids 7-166 or 731-1003 of the Cas9 amino acid sequence set forth in SEQ ID NO:26, or to the corresponding portions in any of the amino acid sequences set forth as SEQ ID NOs:6-816 in PCT/US2017/017255. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to the Cas9 amino acid sequence set forth in SEQ ID NO:26, or to any of the amino acid sequences set forth as SEQ ID NOs:6-816 in PCT/US2017/017255.

In some cases, a suitable Cas9 protein comprises an amino acid sequence having 60% or more amino acid sequence identity to the Cas9 amino acid sequence set forth in SEQ ID NO:26, or to any of the amino acid sequences set forth as SEQ ID NOs:6-816 in PCT/US2017/017255. In some cases, a suitable Cas9 protein comprises an amino acid sequence having 70% or more amino acid sequence identity to the Cas9 amino acid sequence set forth in SEQ ID NO:26, or to any of the amino acid sequences set forth as SEQ ID NOs:6-816. In some cases, a suitable Cas9 protein comprises an amino acid sequence having 75% or more amino acid sequence identity to the Cas9 amino acid sequence set forth in SEQ ID NO:26, or to any of the amino acid sequences set forth as SEQ ID NOs:6-816. In some cases, a suitable Cas9 protein comprises an amino acid sequence having 80% or more amino acid sequence identity to the Cas9 amino acid sequence set forth in SEQ ID NO:26, or to any of the amino acid sequences set forth as SEQ ID NOs:6-816. In some cases, a suitable Cas9 protein comprises an amino acid sequence having 85% or more amino acid sequence identity to the Cas9 amino acid sequence set forth in SEQ ID NO:26, or to any of the amino acid sequences set forth as SEQ ID NOs:6-816 in PCT/US2017/017255. In some cases, a suitable Cas9 protein comprises an amino acid sequence having 90% or more amino acid sequence identity to the Cas9 amino acid sequence set forth in SEQ ID NO:26, or to any of the amino acid sequences set forth as SEQ ID NOs:6-816. In some cases, a suitable Cas9 protein comprises an amino acid sequence having 95% or more amino acid sequence identity to the Cas9 amino acid sequence set forth in SEQ ID NO:26, or to any of the amino acid sequences set forth as SEQ ID NOs:6-816 in PCT/US2017/017255. In some cases, a suitable Cas9 protein comprises an amino acid sequence having 99% or more amino acid sequence identity to the Cas9 amino acid sequence set forth in SEQ ID NO:26, or to any of the amino acid sequences set forth as SEQ ID NOs:6-816 in PCT/US2017/017255. In some cases, a suitable

Cas9 protein comprises an amino acid sequence having 100% amino acid sequence identity to the Cas9 amino acid sequence set forth in SEQ ID NO:26, or to any of the amino acid sequences set forth as SEQ ID NOs:6-816 in PCT/US2017/017255. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a Cas9 protein comprises 4 motifs (as listed in Table 1), at least one with (or each with) amino acid sequences having 75% or more, 80% or more, 85% or more, 90% or more, 95% or more, 99% or more or 100% amino acid sequence identity to each of the 4 motifs listed in Table 1 (SEQ ID NOs:1-4), or to the corresponding portions in any of the amino acid sequences set forth as SEQ ID NOs:6-816 in PCT/US2017/017255.

In some cases, a Cas9 protein is a high fidelity Cas9 protein (see, e.g., Kleinstiver et al. (2016) Nature, 529(7587): 490-495).

In some cases, a suitable Cas9 protein is a Cas9 protein as described in Slaymaker et al. (2016) Science 351: 84. For example, a suitable Cas9 protein can include a Streptococcus pyogenes Cas9 with substitutions of one or more of K810, K848, K855, K1003, and R1060 (where the amino acid numbering is based on the numbering set out in SEQ ID NO:26 (SEQ ID No:5 in PCT/US2017/017255)). For example, a suitable Cas9 protein includes a Streptococcus pyogenes Cas9 with K810A, K1003A, and R1060A substitutions (where the amino acid numbering is based on the numbering set out in SEQ ID NO:26 (SEQ ID No:5 in PCT/US2017/017255)). As another example, a suitable Cas9 protein includes a Streptococcus pyogenes Cas9 with K848A, K1003A, and R1060A substitutions (where the amino acid numbering is based on the numbering set out in SEQ ID NO:5). As another example, a suitable Cas9 protein includes a Streptococcus pyogenes Cas9 with a K855A substitution (where the amino acid numbering is based on the numbering set out in SEQ ID NO:26 (SEQ ID No:5 in PCT/US2017/017255).

Type V and Type VI CRISPR/Cas Endonucleases

In certain embodiments the plasmid(s) complexed with the PRX carriers described herein encode a type V or type VI CRISPR/Cas endonuclease (e.g., Cpf1, C2c1, C2c2, C2c3) and associated guide RNA(s). Type V and type VI CRISPR/Cas endonucleases are a type of class 2 CRISPR/Cas endonuclease. Examples of type V CRISPR/Cas endonucleases include, but are not limited to, Cpf1, C2c1, and C2c3. An example of a type VI CRISPR/Cas endonuclease is C2c2. In some cases, the plasmid encodes a type V CRISPR/Cas endonuclease (e.g., Cpf1, C2c1, C2c3). In some cases, a Type V CRISPR/Cas endonuclease is a Cpf1 protein. In some cases, the plasmid encodes a a type VI CRISPR/Cas endonuclease (e.g., C2c2).

Like type II CRISPR/Cas endonucleases, type V and VI CRISPR/Cas endonucleases form a complex with a corresponding guide RNA. The guide RNA provides target specificity to an endonuclease-guide RNA RNP complex by having a nucleotide sequence (a guide sequence) that is complementary to a sequence (the target site) of a target nucleic acid (as described elsewhere herein). The endonuclease of the complex provides the site-specific activity. In other words, the endonuclease is guided to a target site (e.g., stabilized at a target site) within a target nucleic acid sequence (e.g. genomic DNA) by virtue of its association with the protein-binding segment of the guide RNA.

Examples and guidance related to type V and type VI CRISPR/Cas proteins (e.g., cpf1, C2c1, C2c2, and C2c3 guide RNAs) can be found in the art (see, e.g., Zetsche et al. (2015) Cell, 163(3):759-771; Makarova et al. (2015) Nat. Rev. Microbiol. 13(11): 722-736; Shmakov et al. (2015)Mol. Cell, 60(3): 385-397; and the like).

In some cases, the Type V or type VI CRISPR/Cas endonuclease (e.g., Cpf1, C2c1, C2c2, C2c3) is enzymatically active, e.g., the Type V or type VI CRISPR/Cas polypeptide, when bound to a guide RNA, cleaves a target nucleic acid. In some cases, the Type V or type VI CRISPR/Cas endonuclease (e.g., Cpf1, C2c1, C2c2, C2c3) exhibits reduced enzymatic activity relative to a corresponding wild-type a Type V or type VI CRISPR/Cas endonuclease (e.g., Cpf1, C2c1, C2c2, C2c3), and retains DNA binding activity (e.g., in some cases the endonuclease is a nickase).

In some cases a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs:27-31 (SEQ ID NOs:1088-1092 in PCT/US2017/017255). In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the Cpf1 amino acid sequence set forth in any of SEQ ID NOs:27-31.

In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvC1 domain of the Cpf1 amino acid sequence set forth in any of SEQ ID NOs:27-31. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the Cpf1 amino acid sequence set forth in any of SEQ ID NOs:27-31. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the Cpf1 amino acid sequence set forth in any of SEQ ID NOs:27-31. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the Cpf1 amino acid sequence set forth in any of SEQ ID NOs:27-31.

In some cases, the Cpf1 protein exhibits reduced enzymatic activity relative to a wild-type Cpf1 protein (e.g., relative to a Cpf1 protein comprising the amino acid sequence set forth in any of SEQ ID NOs:27-31), and retains DNA binding activity. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs:27-31; and comprises an amino acid substitution (e.g., a D→A substitution) at an amino acid residue corresponding to amino acid 917 of the Cpf1 amino acid sequence set forth in SEQ ID NO:27. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs:27-31; and comprises an amino acid substitution (e.g., an E→A substitution) at an amino acid residue corresponding to amino acid 1006 of the Cpf1 amino acid sequence set forth in SEQ ID NO:27. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs:27-31; and comprises an amino acid substitution (e.g., a D→A substitution) at an amino acid residue corresponding to amino acid 1255 of the Cpf1 amino acid sequence set forth in SEQ ID NO:27.

In some cases, a suitable Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence set forth in any of SEQ ID NOs:27-31.

In some cases a type V CRISPR/Cas endonuclease is a C2c1 protein (examples include those set forth as SEQ ID NOs:32-39 (SEQ ID NOs:1112-1119 in PCT/US2017/017255). In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c1 amino acid sequence set forth in any of SEQ ID NOs:32-39. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000aa, from 1000 aa to 1100 aa, from 1100 aa to 1200 aa, or from 1200 aa to 1300 aa, of the C2c1 amino acid sequence set forth in any of SEQ ID NOs:32-39.

In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the C2c1 amino acid sequences set forth in any of SEQ ID NOs:32-39). In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the C2c1 amino acid sequence set forth in any of SEQ ID NOs:32-39. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the C2c1 amino acid sequence set forth in any of SEQ ID NOs:32-39. In some cases, a C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the C2c1 amino acid sequence set forth in any of SEQ ID NOs:32-39.

In some cases, the C2c1 protein exhibits reduced enzymatic activity relative to a wild-type C2c1 protein (e.g., relative to a C2c1 protein comprising the amino acid sequence set forth in any of SEQ ID NOs:32-39), and retains DNA binding activity. In some cases, a suitable C2c1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c1 amino acid sequence set forth in any of SEQ ID NOs:32-39.

In some cases a type V CRISPR/Cas endonuclease is a C2c3 protein (examples include those set forth as SEQ ID NOs:40-43 (SEQ ID NOs:1120-1123 in pCT/US2017/017255). In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c3 amino acid sequence set forth in any of SEQ ID NOs:40-43. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c3 amino acid sequence set forth in any of SEQ ID NOs:40-43.

In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the C2c3 amino acid sequence set forth in any of SEQ ID NOs:40-43. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the C2c3 amino acid sequence set forth in any of SEQ ID NOs:40-43. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the C2c3 amino acid sequence set forth in any of SEQ ID NOs:40-43. In some cases, a C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the C2c3 amino acid sequence set forth in any of SEQ ID NOs:40-43.

In some cases, the C2c3 protein exhibits reduced enzymatic activity relative to a wild-type C2c3 protein (e.g., relative to a C2c3 protein comprising the amino acid sequence set forth in any of SEQ ID NOs:40-43), and retains DNA binding activity. In some cases, a suitable C2c3 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c3 amino acid sequence set forth in any of SEQ ID NOs:40-43.

In some cases a type VI CRISPR/Cas endonuclease is a C2c2 protein (examples include those set forth as SEQ ID NOs:44-55 (SEQ ID NOs:1124-1135 in PCT/US2017/017255). In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c2 amino acid sequence set forth in any of SEQ ID NOs:44-55. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the C2c2 amino acid sequence set forth in any of SEQ ID NOs:44-55.

In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of the C2c2 amino acid sequence set forth in any of SEQ ID NOs:44-55. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of the C2c2 amino acid sequence set forth in any of SEQ ID NOs:44-55. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of the C2c2 amino acid sequence set forth in any of SEQ ID NOs:1124-1135. In some cases, a C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI, RuvCII, and RuvCIII domains of the C2c2 amino acid sequence set forth in any of SEQ ID NOs:44-55.

In some cases, the C2c2 protein exhibits reduced enzymatic activity relative to a wild-type C2c2 protein (e.g., relative to a C2c2 protein comprising the amino acid sequence set forth in any of SEQ ID NOs:44-55), and retains DNA binding activity. In some cases, a suitable C2c2 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the C2c2 amino acid sequence set forth in any of SEQ ID NOs:44-55.

PAM Sequence

A wild type class 2 CRISPR/Cas endonuclease (e.g., Cas9 protein) normally has nuclease activity that cleaves a target nucleic acid (e.g., a double stranded DNA (dsDNA)) at a target site defined by complementarity between the guide sequence of the CRISPR/Cas guide RNA and the target nucleic acid. In some cases, site-specific cleavage of the target nucleic acid occurs at locations determined by both (i) base-pairing complementarity between the CRISPR/Cas guide RNA and the target nucleic acid; and (ii) a short motif referred to as the protospacer adjacent motif (PAM) in the target nucleic acid. For example, when the class 2 CRISPR/Cas endonuclease is a wild type Cas9 protein, the PAM sequence that is recognized (e.g., bound) by the Cas9 protein is present on the non-complementary strand (the strand that does not hybridize with the guide sequence of the Cas9 guide RNA) of the target DNA and is adjacent to the target site.

In some cases, (e.g., in some cases where the class 2 CRISPR/Cas endonuclease is an S. pyogenes Cas9 protein) the PAM sequence of the non-complementary strand is 5′-XGG-3′, where X is any DNA nucleotide and X is immediately 3′ of the target sequence of the non-complementary strand of the target DNA. As such, the sequence of the complementary strand that hybridizes with the PAM sequence is 5′-CCY-3′, where Y is any DNA nucleotide and Y is immediately 5′ of the target sequence of the complementary strand of the target DNA. In some such embodiments, X and Y can be complementary and the X-Y base pair can be any base pair (e.g., X=C and Y=G; X=G and Y=C; X=A and Y=T, X=T and Y=A).

In some cases, it may be advantageous to use plasmids encoding different class 2 CRISPR/Cas endonucleases (e.g., Cas9 proteins from various species, type V or type VI CRISPR/Cas endonucleases, and the like) for the subject methods in order to capitalize on various characteristics (e.g., enzymatic characteristics) of the different endonucleases (e.g., for different PAM sequence preferences; for increased or decreased enzymatic activity; for an increased or decreased level of cellular toxicity; to change the balance between NHEJ, homology-directed repair, single strand breaks, double strand breaks, etc.).

Class 2 CRISPR/Cas endonucleases (e.g., Cas9 proteins) from various species can require different PAM sequences in the target DNA, and different types of Class 2 CRISPR/Cas endonucleases (e.g., type II proteins, e.g., Cas9 proteins; type V proteins; type VI proteins; and the like) can have different requirements (e.g., 5′, 3′, complementary strand, non-complementary strand, distance from target sequence, and the like) for the location of the PAM sequence relative to the targeted sequence of the target DNA. Thus, for a particular Class 2 CRISPR/Cas endonuclease of choice, the PAM sequence requirement may be different than the 5′-XGG-3′ sequence described above for the S. pyogenes Cas9 protein.

In some embodiments (e.g., when the Cas9 protein is derived from S. pyogenes or a closely related Cas9 is used), a PAM sequence can be can be 5′-NGG-3′, where N is any nucleotide (see, e.g., Chylinski et al. (2013) RNA Biol. 10(5): 726-737; Jinek et al. (2012) Science, 337(6096): 816-821; and the like). In some embodiments (e.g., when a Cas9 protein is derived from the Cas9 protein of Neisseria meningitidis or a closely related Cas9 is used), the PAM sequence can be 5′-NNNNGANN-3′, 5′-NNNNGTTN-3′, 5′-NNNNGNNT-3′, 5′-NNNNGTNN-3′, 5′-NNNGNTN-3′, or 5′-NNNNGATT-3′, where N is any nucleotide. In some embodiments (e.g., when a Cas9 protein is derived from Streptococcus thermophilus #1 or a closely related Cas9 is used), the PAM sequence can be 5′-NNAGAA-3′, 5′-NNAGGA-3′, 5′-NNGGAA-3′, 5′-NNANAA-3′, or 5′-NNGGGA-3′ where N is any nucleotide. In some embodiments (e.g., when a Cas9 protein is derived from Treponema denticola (TD) or a closely related Cas9 is used), the PAM sequence can be 5′-NAAAAN-3′, 5′-NAAAAC-3′, 5′-NAAANC-3′, 5′-NANAAC-3′, or 5′-NNAAAC-3′, where N is any nucleotide.

The PAM requirements for any given Class 2 CRISPR/Cas endonuclease can be determined using standard, routine, conventional methods, which can include experimental methods and/or in silica analysis of naturally existing sequences from species of interest. For example, as would be known by one of ordinary skill in the art, additional PAM sequences for other Class 2 CRISPR/Cas endonucleases (e.g., Cas9 proteins of different species; type IV CRISPR/Cas endonucleases, type V CRISPR/Cas endonucleases, and the like) can readily be determined using bioinformatic analysis (e.g., analysis of genomic sequencing data) (see, e.g., Mojica et al. (2009) Microbiology, 155(Pt 3): 733-740; Esvelt et al. (2013) Nat. Meth. 10(11): 1116-11121; and the like).

In addition, as known in the art, the PAM-interacting domain of a Class 2 CRISPR/Cas endonuclease (e.g., a Cas9 protein) can be derived from an endonuclease (e.g., Cas9 protein) from a first species, and the PAM sequence can correspond to that domain. Thus, in some cases, a Class 2 CRISPR/Cas endonuclease has a PAM-interacting domain that is derived from (e.g., that is from) a Class 2 CRISPR/Cas endonuclease (e.g., Cas9 protein) of a first species, and other portions of the Class 2 CRISPR/Cas endonuclease (e.g., Cas9 protein) can be derived from (e.g., can be from) a second species.

Guide RNA (for CRISPR/Cas Endonucleases)

A nucleic acid molecule that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to as a “guide RNA” or “CRISPR/Cas guide nucleic acid” or “CRISPR/Cas guide RNA.”

A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

A guide RNA can be referred to by the protein to which it corresponds. For example, when the class 2 CRISPR/Cas endonuclease is a Cas9 protein, the corresponding guide RNA can be referred to as a “Cas9 guide RNA.” Likewise, as another example, when the class 2 CRISPR/Cas endonuclease is a Cpf1 protein, the corresponding guide RNA can be referred to as a “Cpf1 guide RNA.”

In some embodiments, a guide RNA includes two separate nucleic acid molecules: an “activator” and a “targeter” and is referred to as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

Cas9 Guide RNA

A nucleic acid molecule that binds to a Cas9 protein and targets the complex to a specific location within a target nucleic acid is referred to herein as a “Cas9 guide RNA. A Cas9 guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule.

The first segment (targeting segment) of a Cas9 guide RNA includes a nucleotide sequence (a guide sequence) that is complementary to (and therefore hybridizes with) a specific sequence (a target site) within a target nucleic acid (e.g., a target genomic DNA). The protein-binding segment (or “protein-binding sequence”) interacts with (binds to) a Cas9 polypeptide. The protein-binding segment of a subject Cas9 guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid.

A Cas9 guide RNA and a Cas9 protein form a complex (e.g., bind via non-covalent interactions). The Cas9 guide RNA provides target specificity to the complex by including a targeting segment, which includes a guide sequence (a nucleotide sequence that is complementary to a sequence of a target nucleic acid). The Cas9 protein of the complex provides the site-specific activity (e.g., cleavage activity). In other words, the Cas9 protein is guided to a target nucleic acid sequence (e.g. genomic DNA) by virtue of its association with the Cas9 guide RNA.

The “guide sequence” also referred to as the “targeting sequence” of a Cas9 guide RNA can be modified so that the Cas9 guide RNA can target a Cas9 protein to any desired sequence of any desired target nucleic acid, with the exception that the protospacer adjacent motif (PAM) sequence can be taken into account. Thus, for example, a Cas9 guide RNA can have a targeting segment with a sequence (a guide sequence) that has complementarity with (e.g., can hybridize to) a sequence in a nucleic acid in a eukaryotic cell (e.g., genomic DNA).

In some embodiments, a Cas9 guide RNA includes two separate nucleic acid molecules: an “activator” and a “targeter” and is referred to herein as a “dual Cas9 guide RNA”, a “double-molecule Cas9 guide RNA”, or a “two-molecule Cas9 guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

A Cas9 guide RNA comprises a crRNA-like (“CRISPR RNA” I “targeter”/“crRNA”/“crRNA repeat”) molecule and a corresponding tracrRNA-like (“trans-acting CRISPR RNA”/“activator” I “tracrRNA”) molecule. A crRNA-like molecule (targeter) comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator/tracrRNA) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the Cas9 guide RNA. As such, each targeter molecule can be said to have a corresponding activator molecule (which has a region that hybridizes with the targeter). The targeter molecule additionally provides the targeting segment. Thus, a targeter and an activator molecule (as a corresponding pair) hybridize to form a Cas9 guide RNA. The exact sequence of a given crRNA or tracrRNA molecule is characteristic of the species in which the RNA molecules are found. A subject dual Cas9 guide RNA can include any corresponding activator and targeter pair.

The term “activator” or “activator RNA” is used herein to mean a tracrRNA-like molecule (tracrRNA: “trans-acting CRISPR RNA”) of a Cas9 dual guide RNA (and therefore of a Cas9 single guide RNA when the “activator” and the “targeter” are linked together by, e.g., intervening nucleotides). Thus, for example, a Cas9 guide RNA (dgRNA or sgRNA) comprises an activator sequence (e.g., a tracrRNA sequence). A tracr molecule (a tracrRNA) is a naturally existing molecule that hybridizes with a CRISPR RNA molecule (a crRNA) to form a Cas9 dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a Cas9 dual guide RNA (and therefore of a Cas9 single guide RNA when the “activator” and the “targeter” are linked together, e.g., by intervening nucleotides). Thus, for example, a Cas9 guide RNA (dgRNA or sgRNA) comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

A Cas9 guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A Cas9 guide RNA (e.g. a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator) comprises a stretch of nucleotides (a duplex-forming segment) that forms the other half of the dsRNA duplex of the protein-binding segment of the Cas9 guide RNA. In other words, a stretch of nucleotides of the targeter is complementary to and hybridizes with a stretch of nucleotides of the activator to form the dsRNA duplex of the protein-binding segment of a Cas9 guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a Cas9 guide RNA. The particular sequence of a given naturally existing crRNA or tracrRNA molecule is characteristic of the species in which the RNA molecules are found. Examples of suitable activator and targeter are well known in the art.

A Cas9 guide RNA (e.g. a dual guide RNA or a single guide RNA) can be comprised of any corresponding activator and targeter pair. Non-limiting examples of nucleotide sequences that can be included in a Cas9 guide RNA (dgRNA or sgRNA) include sequences set forth in SEQ ID NOs:827-1075 in PCT/US2017/017255, or complements thereof. For example, in some cases, sequences from SEQ ID NOs:827-957 in PCT/US2017/017255 (which are from tracrRNAs) or complements thereof, can pair with sequences from SEQ ID NOs:964-1075 in PCT/US2017/017255 (which are from crRNAs), or complements thereof, to form a dsRNA duplex of a protein binding segment. In some cases, the duplex-forming portion of a guide RNA suitable for use herein comprises the sequence: gttttagagctaGAAAtagcaagttaaaataagg ctagtccgttatcaactt gaaaaagtggcac cgagtcggtgcTTTTTT (SEQ ID NO:5) (SEQ ID NO:1366 in PCT/US2017/017255), or guuuuagagcuaGAAAuagcaaguuaa aauaaggcuaguccguuaucaacuugaaa aaguggcaccgagucggug cUU UUUU (SEQ ID NO:6) (SEQ ID NO:1367 in PCT/US2017/017255).

### Targeting Segment of a Cas9 Guide RNA

A subject guide RNA includes a guide sequence (i.e., a targeting sequence) (a nucleotide sequence that is complementary to a sequence (a target site) in a target nucleic acid). In other words, the targeting segment of a subject guide nucleic acid can interact with a target nucleic acid (e.g., double stranded DNA (dsDNA)) in a sequence-specific manner via hybridization (i.e., base pairing). As such, the nucleotide sequence of the targeting segment may vary (depending on the target) and can determine the location within the target nucleic acid that the Cas9 guide RNA and the target nucleic acid will interact. The targeting segment of a Cas9 guide RNA can be modified (e.g., by genetic engineering)/designed to hybridize to any desired sequence (target site) within a target nucleic acid (e.g., a eukaryotic target nucleic acid such as genomic DNA).

In various embodiments, the targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

In various embodiments, the nucleotide sequence (the targeting sequence, the guide sequence) of the targeting segment that is complementary to a nucleotide sequence (target site) of the target nucleic acid can have a length of 10 nt or more. For example, the targeting sequence of the targeting segment that is complementary to a target site of the target nucleic acid can have a length of 12 nt or more, 15 nt or more, 17 nt or more, 18 nt or more, 19 nt or more, or 20 nt or more. In some cases, the nucleotide sequence (the targeting sequence) of the targeting segment that is complementary to a nucleotide sequence (target site) of the target nucleic acid has a length of 12 nt or more. In some cases, the nucleotide sequence (the targeting sequence) of the targeting segment that is complementary to a nucleotide sequence (target site) of the target nucleic acid has a length of 17 nt or more. In some cases, the nucleotide sequence (the targeting sequence) of the targeting segment that is complementary to a nucleotide sequence (target site) of the target nucleic acid has a length of 18 nt or more.

For example, in certain embodiments, the targeting sequence (guide sequence) of the targeting segment that is complementary to a target sequence of the target nucleic acid can have a length of from 10 to 100 nucleotides (nt) (e.g., from 10 to 90 nt, from 10 to 75 nt, from 10 to 60 nt, from 10 to 50 nt, from 10 to 35 nt, from 10 to 30 nt, from 10 to 25 nt, from 10 to 22 nt, from 10 to 20 nt, from 12 to 100 nt, from 12 to 90 nt, from 12 to 75 nt, from 12 to 60 nt, from 12 to 50 nt, from 12 to 35 nt, from 12 to 30 nt, from 12 to 25 nt, from 12 to 22 nt, from 12 to 20 nt, from 15 to 100 nt, from 15 to 90 nt, from 15 to 75 nt, from 15 to 60 nt, from 15 to 50 nt, from 15 to 35 nt, from 15 to 30 nt, from 15 to 25 nt, from 15 to 22 nt, from 15 to 20 nt, from 17 to 100 nt, from 17 to 90 nt, from 17 to 75 nt, from 17 to 60 nt, from 17 to 50 nt, from 17 to 35 nt, from 17 to 30 nt, from 17 to 25 nt, from 17 to 22 nt, from 17 to 20 nt, from 18 to 100 nt, from 18 to 90 nt, from 18 to 75 nt, from 18 to 60 nt, from 18 to 50 nt, from 18 to 35 nt, from 18 to 30 nt, from 18 to 25 nt, from 18 to 22 nt, or from 18 to 20 nt). In some cases, the targeting sequence of the targeting segment that is complementary to a target sequence of the target nucleic acid has a length of from 15 nt to 30 nt. In some cases, the targeting sequence of the targeting segment that is complementary to a target sequence of the target nucleic acid has a length of from 15 nt to 25 nt. In some cases, the targeting sequence of the targeting segment that is complementary to a target sequence of the target nucleic acid has a length of from 17 nt to 30 nt. In some cases, the targeting sequence of the targeting segment that is complementary to a target sequence of the target nucleic acid has a length of from 17 nt to 25 nt. In some cases, the targeting sequence of the targeting segment that is complementary to a target sequence of the target nucleic acid has a length of from 17 nt to 22 nt. In some cases, the targeting sequence of the targeting segment that is complementary to a target sequence of the target nucleic acid has a length of from 18 nt to 30 nt. In some cases, the targeting sequence of the targeting segment that is complementary to a target sequence of the target nucleic acid has a length of from 18 nt to 25 nt. In some cases, the targeting sequence of the targeting segment that is complementary to a target sequence of the target nucleic acid has a length of from 18 nt to 22 nt. In some cases, the targeting sequence of the targeting segment that is complementary to a target site of the target nucleic acid is 20 nucleotides in length. In some cases, the targeting sequence of the targeting segment that is complementary to a target site of the target nucleic acid is 19 nucleotides in length. In some cases, the targeting sequence of the targeting segment that is complementary to a target site of the target nucleic acid is 18 nucleotides in length. In some cases, the targeting sequence of the targeting segment that is complementary to a target site of the target nucleic acid is 17 nucleotides in length.

In various embodiments, the percent complementarity between the targeting sequence (guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%).

In some cases, the percent complementarity between the targeting sequence of the targeting segment and the target site of the target nucleic acid is 100% over the seven contiguous 5′-most nucleotides of the target site of the target nucleic acid. In some cases, the percent complementarity between the targeting sequence of the targeting segment and the target site of the target nucleic acid is 60% or more over about 20 contiguous nucleotides. In some cases, the percent complementarity between the targeting sequence of the targeting segment and the target site of the target nucleic acid is 100% over the fourteen contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the seven contiguous 5′-most nucleotides of the target site of the target nucleic acid and as low as 0% or more over the remainder.

In some cases, the percent complementarity between the targeting sequence of the targeting segment and the target site of the target nucleic acid is 100% over the 7 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 8 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 9 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 10 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 17 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 18 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over 20 contiguous nucleotides. In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over 17 contiguous nucleotides.

In some cases, the percent complementarity between the targeting sequence of the targeting segment and the target site of the target nucleic acid is 100% over the 7 contiguous 5′-most nucleotides of the target site of the target nucleic acid and as low as 0% or more over the remainder. In such a case, the targeting sequence can be considered to be 7 nucleotides in length. In some cases, the percent complementarity between the targeting sequence of the targeting segment and the target site of the target nucleic acid is 100% over the 8 contiguous 5′-most nucleotides of the target site of the target nucleic acid and as low as 0% or more over the remainder. In such a case, the targeting sequence can be considered to be 8 nucleotides in length. In some cases, the percent complementarity between the targeting sequence of the targeting segment and the target site of the target nucleic acid is 100% over the 9 contiguous 5′-most nucleotides of the target site of the target nucleic acid and as low as 0% or more over the remainder. In such a case, the targeting sequence can be considered to be 9 nucleotides in length. In some cases, the percent complementarity between the targeting sequence of the targeting segment and the target site of the target nucleic acid is 100% over the 10 contiguous 5′-most nucleotides of the target site of the target nucleic acid and as low as 0% or more over the remainder. In such a case, the targeting sequence can be considered to be 10 nucleotides in length. In some cases, the percent complementarity between the targeting sequence of the targeting segment and the target site of the target nucleic acid is 100% over the 11 contiguous 5′-most nucleotides of the target site of the target nucleic acid and as low as 0% or more over the remainder. In such a case, the targeting sequence can be considered to be 11 nucleotides in length. In some cases, the percent complementarity between the targeting sequence of the targeting segment and the target site of the target nucleic acid is 100% over the 12 contiguous 5′-most nucleotides of the target site of the target nucleic acid and as low as 0% or more over the remainder. In such a case, the targeting sequence can be considered to be 12 nucleotides in length. In some cases, the percent complementarity between the targeting sequence of the targeting segment and the target site of the target nucleic acid is 100% over the 13 contiguous 5′-most nucleotides of the target site of the target nucleic acid and as low as 0% or more over the remainder. In such a case, the targeting sequence can be considered to be 13 nucleotides in length. In some cases, the percent complementarity between the targeting sequence of the targeting segment and the target site of the target nucleic acid is 100% over the 14 contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the 17 contiguous 5′-most nucleotides of the target site of the target nucleic acid and as low as 0% or more over the remainder. In such a case, the targeting sequence can be considered to be 17 nucleotides in length. In some cases, the percent complementarity between the targeting sequence of the targeting segment and the target site of the target nucleic acid is 100% over the 18 contiguous 5′-most nucleotides of the target site of the target nucleic acid and as low as 0% or more over the remainder. In such a case, the targeting sequence can be considered to be 18 nucleotides in length.

Examples of various Cas9 proteins and Cas9 guide RNAs (as well as information regarding requirements related to protospacer adjacent motif (PAM) sequences present in targeted nucleic acids) can be found in the art (see, e.g., Jinek et al., (2012) Science, 337(6096):816-821; Chylinski et al. (2013) RNA Biol. 10(5): 726-737; Ma et al., (2013) Biomed Res Int. 2013:270805; Hou et al. (2013) Proc. Natl. Acad. Sci. USA, 110(39): 15644-15649; Jinek et al. (2013) Elife, 2: e00471; Pattanayak et al. (2013) Nat. Biotechnol. 31(9): 839-843; Qi et al. (2013) Cell, 152(5): 1173-1183; Wang et al. (2013) Cell, 153(4): 910-918; Chen et al. (2013) Nucleic Acids Res. 41(20): e19; Cheng et al. (2013) Cell Res. 23(10): 1163-1171; Cho et al. (2013) Genetics, 195(3): 1177-1180; DiCarlo et al. (2013) Nucleic Acids Res. 41(7): 4336-4343; Dickinson et al. (2013) Nat. Meth. 10(10): 1028-1034; Ebina et al. (2013) Sci Rep. 3: 2510; Fujii et. al. (2013) Nucleic Acids Res. 41(20): e187; Hu et al. (2013) Cell Res. 23(11): 1322-1325; Jiang et al. (2013) Nucleic Acids Res. 41(20): e188; Larson et al. (2013) Nat. Protoc. 8(11): 2180-2196; Mali et al. (2013) Nat. Meth. 10(10): 957-963; Nakayama et al. (2013) Genesis, 51(12): 835-843; Ran et al. (2013) Nat. Protoc. 8(11): 2281-308; Ran et al. (2013) Cell, 154(6): 1380-1389; Upadhyay et al. (2013) G3 (Bethesda) 3(12): 2233-2238; Walsh et al. (2013) Proc. Natl. Acad. Sci. USA, 110(39): 15514-15515; Yang et al. (2013) Cell, 154(6): 1370-1379; Briner et al. (2014)Mol. Cell, 56(2): 333-339; and U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

### Guide RNAs Corresponding to Type V and Type VI CRISPR/Cas Endonucleases (e.g., Cpf1 Guide RNA)

A guide RNA that binds to a type V or type VI CRISPR/Cas protein (e.g., Cpf1, C2c1, C2c2, C2c3), and targets the complex to a specific location within a target nucleic acid is referred to herein generally as a “type V or type VI CRISPR/Cas guide RNA”. An example of a more specific term is a “Cpf1 guide RNA.”

A type V or type VI CRISPR/Cas guide RNA (e.g., cpf1 guide RNA) can have a total length of from 30 nucleotides (nt) to 200 nt, e.g., from 30 nt to 180 nt, from 30 nt to 160 nt, from 30 nt to 150 nt, from 30 nt to 125 nt, from 30 nt to 100 nt, from 30 nt to 90 nt, from 30 nt to 80 nt, from 30 nt to 70 nt, from 30 nt to 60 nt, from 30 nt to 50 nt, from 50 nt to 200 nt, from 50 nt to 180 nt, from 50 nt to 160 nt, from 50 nt to 150 nt, from 50 nt to 125 nt, from 50 nt to 100 nt, from 50 nt to 90 nt, from 50 nt to 80 nt, from 50 nt to 70 nt, from 50 nt to 60 nt, from 70 nt to 200 nt, from 70 nt to 180 nt, from 70 nt to 160 nt, from 70 nt to 150 nt, from 70 nt to 125 nt, from 70 nt to 100 nt, from 70 nt to 90 nt, or from 70 nt to 80 nt). In some cases, a type V or type VI CRISPR/Cas guide RNA (e.g., cpf1 guide RNA) has a total length of at least 30 nt (e.g., at least 40 nt, at least 50 nt, at least 60 nt, at least 70 nt, at least 80 nt, at least 90 nt, at least 100 nt, or at least 120 nt).

In some cases, a Cpf1 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt.

Like a Cas9 guide RNA, a type V or type VI CRISPR/Cas guide RNA (e.g., cpf1 guide RNA) can include a target nucleic acid-binding segment and a duplex-forming region (e.g., in some cases formed from two duplex-forming segments, i.e., two stretches of nucleotides that hybridize to one another to form a duplex).

The target nucleic acid-binding segment of a type V or type VI CRISPR/Cas guide RNA (e.g., cpf1 guide RNA) can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some cases, the target nucleic acid-binding segment has a length of 23 nt. In some cases, the target nucleic acid-binding segment has a length of 24 nt. In some cases, the target nucleic acid-binding segment has a length of 25 nt.

The guide sequence of a type V or type VI CRISPR/Cas guide RNA (e.g., cpf1 guide RNA) can have a length of from 15 nt to 30 nt (e.g., 15 to 25 nt, 15 to 24 nt, 15 to 23 nt, 15 to 22 nt, 15 to 21 nt, 15 to 20 nt, 15 to 19 nt, 15 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt.

The guide sequence of a type V or type VI CRISPR/Cas guide RNA (e.g., cpf1 guide RNA) can have 100% complementarity with a corresponding length of target nucleic acid sequence. The guide sequence can have less than 100% complementarity with a corresponding length of target nucleic acid sequence. For example, the guide sequence of a type V or type VI CRISPR/Cas guide RNA (e.g., cpf1 guide RNA) can have 1, 2, 3, 4, or 5 nucleotides that are not complementary to the target nucleic acid sequence. For example, in some cases, where a guide sequence has a length of 25 nucleotides, and the target nucleic acid sequence has a length of 25 nucleotides, in some cases, the target nucleic acid-binding segment has 100% complementarity to the target nucleic acid sequence. As another example, in some cases, where a guide sequence has a length of 25 nucleotides, and the target nucleic acid sequence has a length of 25 nucleotides, in some cases, the target nucleic acid-binding segment has 1 non-complementary nucleotide and 24 complementary nucleotides with the target nucleic acid sequence. As another example, in some cases, where a guide sequence has a length of 25 nucleotides, and the target nucleic acid sequence has a length of 25 nucleotides, in some cases, the target nucleic acid-binding segment has 2 non-complementary nucleotides and 23 complementary nucleotides with the target nucleic acid sequence.

The duplex-forming segment of a type V or type VI CRISPR/Cas guide RNA (e.g., cpf1 guide RNA) (e.g., of a targeter RNA or an activator RNA) can, in some cases, have a length of from 15 nt to 25 nt (e.g., 15 nt, 16 nt, 17 nt, 18 nt, 19 nt, 20 nt, 21 nt, 22 nt, 23 nt, 24 nt, or 25 nt).

In some cases, the RNA duplex of a type V or type VI CRISPR/Cas guide RNA (e.g., cpf1 guide RNA) can have a length of from 5 base pairs (bp) to 40 bp (e.g., from 5 to 35 bp, 5 to 30 bp, 5 to 25 bp, 5 to 20 bp, 5 to 15 bp, 5-12 bp, 5-10 bp, 5-8 bp, 6 to 40 bp, 6 to 35 bp, 6 to 30 bp, 6 to 25 bp, 6 to 20 bp, 6 to 15 bp, 6 to 12 bp, 6 to 10 bp, 6 to 8 bp, 7 to 40 bp, 7 to 35 bp, 7 to 30 bp, 7 to 25 bp, 7 to 20 bp, 7 to 15 bp, 7 to 12 bp, 7 to 10 bp, 8 to 40 bp, 8 to 35 bp, 8 to 30 bp, 8 to 25 bp, 8 to 20 bp, 8 to 15 bp, 8 to 12 bp, 8 to 10 bp, 9 to 40 bp, 9 to 35 bp, 9 to 30 bp, 9 to 25 bp, 9 to 20 bp, 9 to 15 bp, 9 to 12 bp, 9 to 10 bp, 10 to 40 bp, 10 to 35 bp, 10 to 30 bp, 10 to 25 bp, 10 to 20 bp, 10 to 15 bp, or 10 to 12 bp).

As an example, a duplex-forming segment of a Cpf1 guide RNA can comprise a nucleotide sequence selected from (5′ to 3′): AAUUUCUACUGUUGUAGAU (SEQ ID NO:7), AAUUUCUGCUGUUGCAGAU (SEQ ID NO:8), AAUUUCCACUGUUGUGGAU (SEQ ID NO:9), AAUUCCUACUGUUGUAGGU (SEQ ID NO:10), AAUUUCUACUAUUGUAGAU (SEQ ID NO:11), AAUUUCUACUGCUGUAGAU (SEQ ID NO:12), AAUUUCUACUUUGUAGAU (SEQ ID NO:13), and AAUUUCUACUUGUAGAU (SEQ ID NO:14). The guide sequence can then follow (5′ to 3′) the duplex forming segment.

A non-limiting example of an activator RNA (e.g. tracrRNA) of a C2c1 guide RNA (dual guide or single guide) is an RNA that includes the nucleotide sequence GAAUUUUUCAACGGGUGUGCCAAUGGCCACUUUCCAGGUGGCAAAGCCCGUUG A GCUUCUCAAAAAG (SEQ ID NO:15). In some cases, a C2c1 guide RNA (dual guide or single guide) is an RNA that includes the nucleotide sequence In some cases, a C2c1 guide RNA (dual guide or single guide) is an RNA that includes the nucleotide sequence GUCUAGAGGACAGAAUUUUUCAACGGGUGUGCCAAUGGCCACUUUCCAGGUG GC AAAGCCCGUUGAGCUUCUCAAAAAG (SEQ ID NO:16). In some cases, a C2c1 guide RNA (dual guide or single guide) is an RNA that includes the nucleotide sequence UCUAGAGGACAGAAUUUUUCAACGGGUGUGCCAAUGGCCACUUUCCAGGUGGC A AAGCCCGUUGAGCUUCUCAAAAAG (SEQ ID NO:17). A non-limiting example of an activator RNA (e.g. tracrRNA) of a C2c1 guide RNA (dual guide or single guide) is an RNA that includes the nucleotide sequence ACUUUCCAGGCAAAGCCCGU UGAGCUUCUCAAAAAG (SEQ ID NO:18). In some cases, a duplex forming segment of a C2c1 guide RNA (dual guide or single guide) of an activator RNA (e.g. tracrRNA) includes the nucleotide sequence AGCUUCUCA (SEQ ID NO:19) or the nucleotide sequence GCUUCUCA (SEQ ID NO:20) (the duplex forming segment from a naturally existing tracrRNA.

A non-limiting example of a targeter RNA (e.g. crRNA) of a C2c1 guide RNA (dual guide or single guide) is an RNA with the nucleotide sequence CUGAGAAGUGGCACNNNNNNN (SEQ ID NO:21), where the Ns represent the guide sequence, that will vary depending on the target sequence, and although 20 Ns are depicted a range of different lengths are acceptable. In some cases, a duplex forming segment of a C2c1 guide RNA (dual guide or single guide) of a targeter RNA (e.g. crRNA) includes the nucleotide sequence CUGAGAAGUGGCAC (SEQ ID NO:22) or includes the nucleotide sequence CUGAGAAGU (SEQ ID NO:23) or includes the nucleotide sequence UGAGAAGUGGCAC (SEQ ID NO:24) or includes the nucleotide sequence UGAGAAGU (SEQ ID NO:25).

Examples and guidance related to type V or type VI CRISPR/Cas endonucleases and guide RNAs (as well as information regarding requirements related to protospacer adjacent motif (PAM) sequences present in targeted nucleic acids) can be found in the art (see, e.g., Zetsche et al. (2015) Cell, 163(3): 759-771; Makarova et al. (2015) Nat. Rev. Microbiol. 13(11): 722-736; Shmakov et al. (2015)Mol. Cell. 60(3): 385-397, and the like).

SE-Mediated Inhibitory RNA Delivery to the Brain and CNS.

It was also a surprising discovery that the synthetic exosomes described herein can be used to effectively facilitate passage of “SE encapsulated” inhibitory RNAs (e.g., siRNA, shRNA), typically as a DNA encoding the inhibitory RNA, where often such DNA comprise a vector that is capable of transcribing the inhibitory RNA, antibodies across the blood brain barrier. Illustrative inhibitory RNAs include, but are not limited to inhibitory RNAs useful for the treatment neurodegenerative diseases (e.g., Alzheimer’ disease, amyloid-related mild cognitive impairment (MCI), Huntington's disease (HD), amyotrophic lateral sclerosis (ALS), Parkinson's disease (PD), and the like).

Alzheimer's Disease.

Tau and amyloid precursor protein (APP) are key proteins in the pathogenesis of sporadic and inherited Alzheimer's disease. Thus, developing ways to inhibit production of these proteins is of great research and therapeutic interest. The selective silencing of mutant alleles, moreover, represents an attractive strategy for treating inherited dementias and other dominantly inherited disorders.

Miller et al. (2004) Nucleic Acids Res., 32(2): 661-668, describe an efficient method for producing small interfering RNA (siRNA) against essentially any targeted region of a gene. This approach was then used to develop siRNAs that display optimal allele-specific silencing against a well-characterized tau mutation (V337M) and the most widely studied APP mutation (APPsw). The allele-specific RNA duplexes identified by this method then served as templates for constructing short hairpin RNA (shRNA) plasmids that successfully silenced mutant tau or APP alleles.

Other studies have suggested the use of RNAi to inhibit c-SCR, GGA3 adaptor protein, acyl-coenzyme A cholesterol acyltransferase (ACAT-1), and/or tau can be used for the treatment of Alzheimer's disease (see, e.g., Chen et al. (2013) Drug Design Develop. & Therap., 7: 117-115).

The SEs described herein can readily be utilized to deliver these and other shRNA plasmids across the blood brain barrier for the treatment of Alzheimer's disease.

Amyotrophic Lateral Sclerosis (ALS)

Amyotrophic lateral sclerosis (ALS) is a progressive fatal, neurodegenerative disease caused by the degeneration of motor neurons. Although ALS lacks a clear genetic cause, approximately 20% of familial ALS cases are associated with mutations in the superoxide dismutase (SOD1) gene. Kubodera et al. (2010) Hum. Gene Therap., 22(1): doi.org/10.1089/hum.2010.054) developed a therapeutic strategy for ALS where they used an intravenous injection of AAV8 vector to knock down the mutant SOD1 allele using small hairpin RNA (shRNA) while simultaneously expressing functional wild-type SOD1 cDNA.

Without being bound to a particular theory, it is believed the SEs described herein can be used to deliver SOD1 inhibitory RNAs (or constructs encoding SOD1 inhibitory RNAs) for the treatment of ALS.

Huntington's Disease.

It has been demonstrated that a sole injection of cholesterol-conjugated small interfering RNA duplexes (cc-siRNA) targeting huntingtin (Htt) gene into the adult striatum of a viral transgenic mouse model of HD silences mutant Htt, attenuates neuronal pathology, and delays the unusual behavioral phenotype observed in the mouse. In a study by DiFiglia and colleagues, for example, an adeno-associated virus containing either wild-type (18 CAG) or expanded (100 CAG) Htt cDNA encoding Htt 1-400 and siRNA were injected into a mouse striata (see, e.g., DiFiglia et al. (2007) Proc. Natl. Acad. Sci. USA, 104(43): 17204-17296). It was observed that treatment of the mice that had the mutant Htt with cc-siRNA-Htt prolonged survival of striatal neurons, lowered neuropil aggregates, and diminished inclusion size. siRNA reduces the production of mutant Htt protein and silences its expression via RNA interference.

Accordingly, without being bound to a particular theory, it is believed that deliver of inhibitory RNAs (or DNAs encoding inhibitory RNAs) targeting the Htt gene can be used for the treatment of Huntington's disease. Thus, in various embodiments SEs containing Htt inhibitory RNAs are provided.

Parkinson's Disease.

Recent evidence has shown that IRAK4 is a crucial regulator in the body's innate immune response, the body's first line of defense against foreign pathogens activation of which leads to the production of pro-inflammatory cytokines.

As its abnormal function in innate immune cells is implicated in the development of chronic inflammatory and autoimmune diseases, IRAK4 inhibitors have been regarded as the next generation of anti-inflammatory treatments for autoimmune conditions, including rheumatoid arthritis, inflammatory bowel disease, psoriasis, lupus, and the like. However transport of IRAK4 inhibitors across the blood brain barrier and blood-nerve barriers would allow IRAK4 inhibitors to target neuroinflammatory diseases of the central nervous system. Such disease include, inter alia, such as Parkinson's disease, Alzheimer's disease, multiple sclerosis, amyotrophic lateral sclerosis, and diabetic peripheral neuropathy.

Inhibitory RNAs targeting IRAK4 (or DNAs encoding such inhibitory RNAs) packaged in the SEs described herein can readily cross the blood brain barrier, and it is believed such compositions can be used for the treatment of Parkinson's disease. Similarly, it is believed inhibitory RNAs targeting α-synuclein can also be used in the treatment of Huntington's disease.

Accordingly, in certain embodiments, SEs containing inhibitory RNAs or nucleic acids encoding inhibitory RNAs targeting IRAK4 or a synuclein are contemplated.

Similarly humanized monoclonal antibodies such as PRX002 directed against α-synuclein aggregates could be delivered more effectively to the brain by SE's in PD.

Niemann Pick Disease

Patients with types A and B Niemann-Pick disease (NPD) have an inherited deficiency of acid sphingomyelinase (ASM) activity. The clinical spectrum of this disorder ranges from the infantile, neurological form that results in death by 3 years of age (type A NPD) to the non-neurological form (type B NPD) that is compatible with survival into adulthood. Intermediate cases also have been reported, and the disease is best thought of as a single entity with a spectrum of phenotypes. ASM deficiency is panethnic, but appears to be more frequent in individuals of Middle Eastern and North African descent. Current estimates of the disease incidence range from approximately 0.5 to 1 per 100,000 births. However, these approximations likely under estimate the true frequency of the disorder since they are based solely on cases referred to biochemical testing laboratories for enzymatic confirmation.

The gene encoding ASM (SMPD1) has been studied extensively; it resides within an imprinted region on chromosome 11, and is preferentially expressed from the maternal chromosome. Over 100 SMPD1 mutations causing ASM-deficient NPD have been described, and some useful genotype-phenotype correlations have been made. Based on these findings, DNA-based carrier screening has been implemented in the Ashkenazi Jewish community. ASM ‘knockout’ mouse models also have been constructed and used to investigate disease pathogenesis and treatment.

Based on these studies in the mouse model, an enzyme replacement therapy clinical trial has recently begun in adult patients with non-neurological ASM-deficient NPD. In view of these findings, in certain embodiments, it is contemplated to package acid sphingomyelinase (ASM) in the synthetic exosomes described herein, for systemic, and in particular, for delivery to the central nervous system.

Preparation of Inhibitory RNAs.

In various embodiments the composition contained in the SE includes double-stranded DNA (dsDNA) that encodes for a promoter region and for siRNA, or a promoter region and short hairpin RNA (shRNA). In certain embodiments the SE includes double-stranded DNA (dsDNA) that encodes for a promoter region and for a siRNA (long or short dsRNA), or alternatively, a promoter region and short hairpin RNA (shRNA).

dsDNA which encodes for a promoter region and for siRNA sequences that are complementary to the nucleotide sequence of the target gene can be prepared using standard methods well known to those of skill in the art. For example, the siRNA nucleotide sequence can be obtained from the siRNA Selection Program, Whitehead Institute for Biomedical Research, Massachusetts Institute of Technology, Cambridge, Mass. (//jura.wi.mit.edu) after supplying the Accession Number or GI number from the National Center for Biotechnology Information website (www.ncbi.nlm.nih.gov). The Genome Database (www.gdb.org) provides the nucleic acid sequence link which can be used as the National Center for Biotechnology Information accession number. Preparation to order of dsDNA, that encodes for the U6 promoter and for siRNA, is commercially available (Promega, Madison, Wis.). Determination of the appropriate sequences can readily be accomplished using the USPHS, NIH genetic sequence data bank. Alternatively, dsRNA containing appropriate siRNA sequences can be ascertained using the strategy of Miyagishi and Taira (2003) Nat. Biotechnol. 20: 497-500. In certain embodiments DsRNA may be up to 800 base pairs long (Diallo et al. (2003) Oligonucleotides 13(5): 381-392). In certain embodiments the dsRNA may have a hairpin structure (see, e.g., US Patent Pub. No: 2004/0058886). Determination of siRNA sequences optionally is also determined using the Promega algorithm (www.promega.com/sirnadesigner). Invitrogen provides another commercially available RNAi designer algorithm (see, e.g., //maidesigner.invitrogen.com/maiexpress/), and the like.

The foregoing inhibitory RNAs are illustrative and non-limiting. Using the teachings provided herein numerous other inhibitory RNAs, or nucleic acids (e.g., DNAs) encoding inhibitory RNAs can readily be provided and incorporated into the SEs described herein.

Similarly in various embodiments miRNA (e.g., miRNA that are small ˜22 nucleotide long non-coding RNA molecules) would be used for beneficial effects in CNS disorders such as AD.

### Kits.

In certain embodiments kits for the delivery of a therapeutic moiety (e.g., sAPPα) to the brain are provided. Typically, such kit will comprise a container containing synthetic exosomes (SEs), containing the therapeutic moiety. In certain embodiments the synthetic exosomes can be provided in a unit dosage formulation (e.g., vial, tablet, caplet, patch, etc.) and/or may be optionally combined with one or more pharmaceutically acceptable excipients.

In addition, in certain embodiments, the kits optionally include labeling and/or instructional materials providing directions (i.e., protocols) for the use of the synthetic exosomes described herein. Thus, for example, the kit may contain directions for the use of the synthetic exosomes comprising a therapeutic moiety in the treatment of dementia, mild cognitive impairment, Alzheimer's disease, Amyotrophic lateral sclerosis (ALS), Parkinson's disease, cerebral amyloid angiopathy, and the like as well as for Traumatic brain injury (TBI) and Stroke therapy or for treatment of a brain cancer. In various embodiments the instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

invention.

### EXAMPLES

The following examples are offered to illustrate, but not to limit the claimed

### Example 1

### Encapsulation of a Hydrophobic Small Molecule

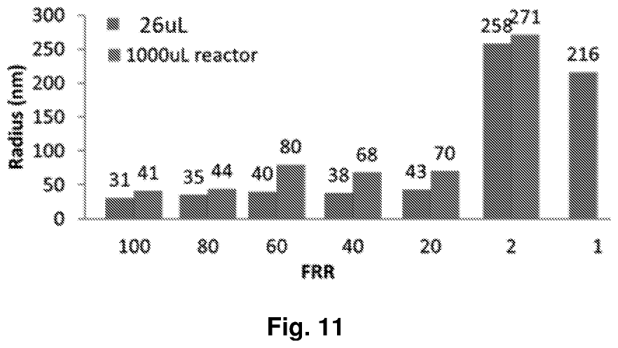

For encapsulation of a hydrophobic small molecule we used a lipid mixture of 2:2:1 (DTPG:DDPA:CH) with a Span-80 concentration of 5% w/w and obtained a zeta potential of around −15 mV. The particle size and flow rate ratio relationship is shown in FIG. 11.

In general, the faster the aqueous flow rate in relationship with the organic stream the smaller the particle size of the SE

### Example 2

### Encapsulation of a Protein

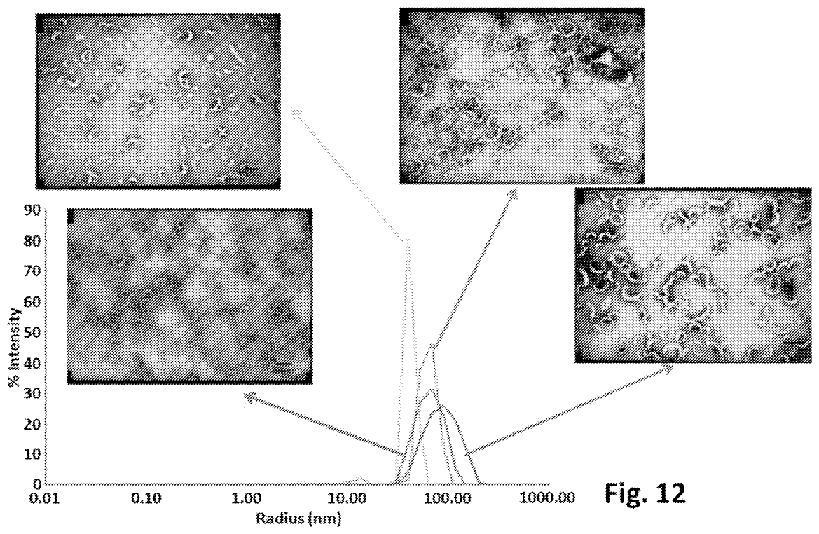

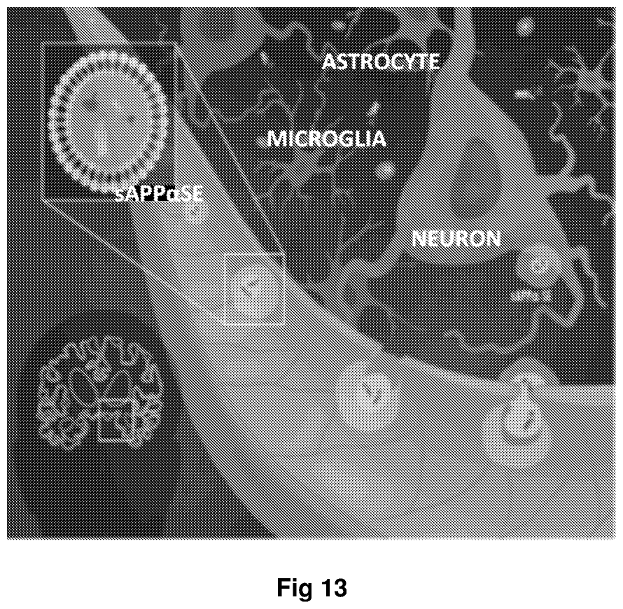

For encapsulation of a protein with molecular weight of 80JDa we used a lipid mixture of 2:2:1 (DHPG:DHPC:CH) with Span-80 concentration of 10% w/w and obtained a zeta potential of around +5 mV. The particle size and flow rate ratio relationship is shown in FIG. 12. The brighter green is a FRR=100, darker green FRR=80, red FRR=25 and blue FRR=20. In general, the faster the aqueous flow rate in relationship with the organic stream the smaller the particle size of the SE.

### Example 3

### Encapsulation of Cas9 and/or IDUA

Table 7, below illustrates the synthetic exosome characteristics for microfluidic-synthesized synthetic exosomes encapsulating IDUA or Cas9.

TABLE 7
Synthetic exosome (SE) characteristics for SEs encapuslting
IDUA or Cas9. Shown are diameter (Φ), zeta potential
(ξ), and encapsulation efficiency (ee).

| | Avg Φ | Std. | ξ (mV) | ee |
|---|---|---|---|---|
| Sample ID | (nm) | Dev. | estimated | (%) |
| SE(ξ+)-IDUA | 110 | 20 | 42 | 3 |
| SE(ξ−)-IDUA | 107 | 6 | −40 | 8 |
| SE(ξ+)-Cas9 | 81 | 6 | 46 | 25 |
| SE(ξ−)-Cas9 | 110 | 8 | −36 | 15 |

Without being bound to a particular theory, the difference in the encapsulation efficiency between IDUA and Cas9 is believed to be due to the stability of the enzymes in aqueous solution.

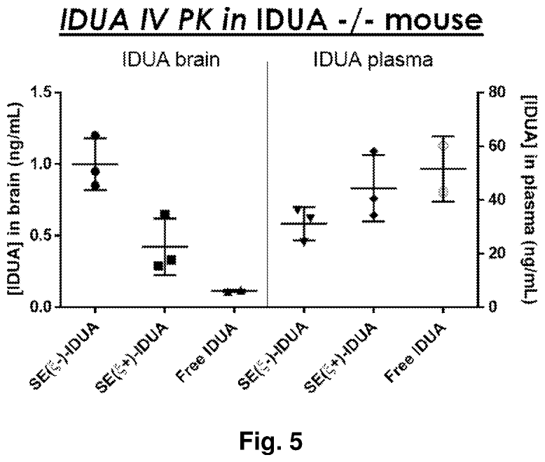

FIG. 5 shows that SE-IDUA (−) particles show ˜10 fold increase in brain 10 levels of IDUA compared to Free IDUA, while Table 8 shows the ratio of brain to plasma IDUA and % IDUA in brain.

TABLE 8
Ratio of brain to plasma IDUA and % IDUA in brain.

| | | | % IDUA |
|---|---|---|---|
| Sample ID | Brain:Plasma | Sample ID | in Brain |
| SE(ξ−)-IDUA | 1:8 | SE(ξ−)-IDUA | 1.9 |
| SE(ξ+)-IDUA | 1:30 | SE(ξ+)-IDUA | 0.8 |
| Free IDUA | 1:120 | Free IDUA | 0.2 |

Table 9 illustrates SE-Cas-9 characterization and IV brain levels.

TABLE 9
Synthetic exosome (SE) characterization and brain levels.

| | Avg Φ | | Ee | Cas9 | % |
|---|---|---|---|---|---|
| | (nm) | ξ (mV) | (%) | (ng) | brain |
| Se-Cas9(−) | 110 | −36 | >15 | 416 | 4.2 |
| Se-Cas9(+) | 81 | 46 | >15 | 330 | 3.3 |

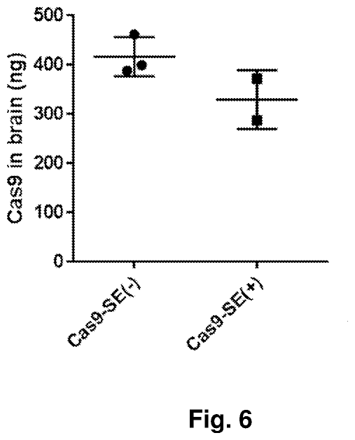

FIG. 6 shows that SE-Cas9(−) has greater brain permeability than SE-Cas9 (+) particles.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent 5 applications cited herein are hereby incorporated by reference in their entirety for all purposes.

| SEQUENCE LISTING | |
|---|---|
| S. pyrogenes Cas9 | |
| Sequence ID NO: 26 | |
| 10 20 30 40 50 | |
| MDKKYSIGLD IGINSVGWAV ITDEYKVPSK KFKVLGNTDR HSIKKNLIGA | |
| 60 70 80 90 100 | |
| LLFDSGETAE ATRLKRTARR RYTRRKNRIC YLQEIFSNEM AKVDDSFFHR | |
| 110 120 130 140 150 | |
| LEESFLVEED KKHERHPIFG NIVDEVAYHE KYPTIYHLRK KLVDSTDKAD | |
| 160 170 180 190 200 | |
| LRLIYLALAH MIKFRGHFLI EGDLNPDNSD VDKLFIQLVQ TYNQLFEENP | |
| 210 220 230 240 250 | |
| INASGVDAKA ILSARLSKSR RLENLIAQLP GEKKNGLFGN LIALSLGLTP | |
| 260 270 280 290 300 | |
| NFKSNFDLAE DAKLQLSKDT YDDDLDNLLA QIGDQYADLF LAAKNLSDAI | |
| 310 320 330 340 350 | |
| LLSDILRVNT EITKAPLSAS MIKRYDEHHQ DLTLLKALVR QQLPEKYKEI | |
| 360 370 380 390 400 | |
| FFDQSKNGYA GYIDGGASQE EFYKFIKPIL EKMDGTEELL VKLNREDLLR | |
| 410 420 430 440 450 | |
| KORTFDNGSI PHQIHLGELH AILRRQEDFY PFLKDNREKI EKILTFRIPY | |
| 460 470 480 490 500 | |
| YVGPLARGNS RFAWMTRKSE ETITPWNFEE VVDKGASAQS FIERMTNFDK | |
| 510 520 530 540 550 | |
| NLPNEKVLPK HSLLYEYFTV YNELTKVKYV TEGMRKPAFL SGEQKKAIVD | |
| 560 570 580 590 600 | |
| LLFKTNRKVT VKQLKEDYFK KIECFDSVEI SGVEDRENAS LGTYHDLLKI | |
| 610 620 630 640 650 | |
| IKDKDFLDNE ENEDILEDIV LTLTLFEDRE MIEERLKTYA HLFDDKVMKQ | |
| 660 670 680 690 700 | |
| LKRRRYTGWG RLSRKLINGI RDKQSGKTIL DFLKSDGFAN RNFMQLIHDD | |
| 710 720 730 740 750 | |
| SLTFKEDIQK AQVSGOGDSL HEHIANLAGS PAIKKGILQT VKVVDELVKV | |
| 760 770 780 790 800 | |
| MGRHKPENIV IEMARENQTT QKGQKNSRER MKRIEEGIKE LGSQILKEHP | |
| 810 820 830 840 850 | |
| VENTQLQNEK LYLYYLONGR DMYVDQELDI NRLSDYDVDH IVPQSFLKDD | |
| 860 870 880 890 900 | |
| SIDNKVLTRS DKNRGKSDNV PSEEVVKKMK NYWRQLLNAK LITQRKFDNL | |
| 910 920 930 940 950 | |
| TKAERGGLSE LDKAGFIKRQ LVETROITKH VAQILDSRMN TKYDENDKLI | |
| 960 970 980 990 1000 | |
| REVKVITLKS KLVSDFRKDF QFYKVREINN YHHAHDAYLN AVVGTALIKK | |
| 1010 1020 1030 1040 1050 | |
| YPKLESEFVY GDYKVYDVRK MIAKSEQEIG KATAKYFFYS NIMNFFKTEI | |
| 1060 1070 1080 1090 1100 | |
| TLANGEIRKR PLIEINGETG EIVWDKGRDF ATVRKVLSMP QVNIVKKTEV | |
| 1110 1120 1130 1140 1150 | |
| QTGGFSKESI LPKRNSDKLI ARKKDWDPKK YGGFDSPTVA YSVLVVAKVE | |
| 1160 1170 1180 1190 1200 | |
| KGKSKKLKSV KELLGITIME RSSFEKNPID FLEAKGYKEV KKDLIIKLPK | |
| 1210 1220 1230 1240 1250 | |
| YSLFELENGR KRMLASAGEL QKGNELALPS KYVNFLYLAS HYEKLKGSPE | |
| 1260 1270 1280 1290 1300 | |
| DNEQKOLFVE QHKHYLDEII EQISEFSKRV ILADANLDKV LSAYNKHRDK | |
| 1310 1320 1330 1340 1350 | |
| PIREQAENII HLFTLINLGA PAAFKYFDTT IDRKRYTSTK EVLDATLIHQ | |
| 1360 | |
| SITGLYETRI DLSQLGGD | |
| (1088 in PCT/US2017/017255) | |
| Francisella tularensis | |
| SEQ ID NO: 27 | |
| MSIYQEFVNK YSLSKTLRFE LIPQGKTLEN IKARGLILDD EKRAKDYKKA KQIIDKYHQF | 60 |
| FIEEILSSVC ISEDLLONYS DVYFKLKKSD DDNLQKDFKS AKDTIKKQIS EYIKDSEKFK | 120 |
| NLFNQNLIDA KKGQESDLIL WLKQSKDNGI ELFKANSDIT DIDEALEIIK SFKGWTTYFK | 180 |
| GFHENRKNVY SSNDIPTSII YRIVDDNLPK FLENKAKYES LKDKAPEAIN YEQIKKDLAE | 240 |
| ELTFDIDYKT SEVNQRVFSL DEVFEIANFN NYLNQSGITK FNTIIGGKFV NGENTKRKGI | 300 |
| NEYINLYSQQ INDKTLKKYK MSVLFKQILS DTESKSFVID KLEDDSDVVT TMQSFYEQIA | 360 |
| AFKTVEEKSI KETLSLLFDD LKAQKLDLSK IYFKNDKSLT DLSQQVFDDY SVIGTAVLEY | 420 |
| ITQQIAPKNL DNPSKKEQEL IAKKTEKAKY LSLETIKLAL EEFNKHRDID KQCRFEEILA | 480 |
| NFAAIPMIFD EIAQNKDNLA QISIKYQNQG KKDLLQASAE DDVKAIKDLL DQTNNLLHKL | 540 |
| KIFHISQSED KANILDKDEH FYLVFEECYF ELANIVPLYN KIRNYITQKP YSDEKFKLNF | 600 |
| ENSTLANGWD KNKEPDNTAI LFIKDDKYYL GVMNKKNNKI FDDKAIKENK GEGYKKIVYK | 660 |
| LLPGANKMLP KVFFSAKSIK FYNPSEDILR IRNHSTHTKN GSPQKGYEKF EFNIEDCRKF | 720 |
| IDFYKQSISK HPEWKDFGFR FSDTQRYNSI DEFYREVENQ GYKLTFENIS ESYIDSVVNQ | 780 |
| GKLYLFQIYN KDFSAYSKGR PNLHTLYWKA LFDERNLQDV VYKLNGEAEL FYRKQSIPKK | 840 |
| ITHPAKEAIA NKNKDNPKKE SVFEYDLIKD KRFTEDKFFF HCPITINFKS SGANKENDEI | 900 |
| NLLLKEKAND VHILSIDRGE RHLAYYTLVD GKGNIIKQDT FNIIGNDRMK TNYHDKLAAI | 960 |
| EKDRDSARKD WKKINNIKEM KEGYLSQVVH EIAKLVIEYN AIVVFEDLNF GFKRGRFKVE | 1020 |
| KOVYQKLEKM LIEKLNYLVF KDNEFDKTGG VLRAYQLTAP FETFKKMGKQ TGIIYYVPAG | 1080 |
| FTSKICPVTG FVNQLYPKYE SVSKSQEFFS KFDKICYNLD KGYFEFSFDY KNFGDKAAKG | 1140 |
| KWTIASFGSR LINFRNSDKN HNWDTREVYP TKELEKLLKD YSIEYGHGEC IKAAICGESD | 1200 |
| KKFFAKLTSV LNTILQMRNS KTGTELDYLI SPVADVNGNF FDSRQAPKNM PQDADANGAY | 1260 |
| HIGLKGLMLL GRIKNNQEGK KLNLVIKNEE YFEFVONRNN | 1300 |
| (1089 in PCT/US2017/017255) | |
| SEQ ID NO: 28 | |
| MTQFEGFTNL YQVSKTLRFE LIPQGKTLKH IQEQGFIEED KARNDHYKEL KPIIDRIYKT | 60 |
| YADQCLQLVQ LDWENLSAAI DSYRKEKTEE TRNALIEEQA TYRNAIHDYF IGRTDNLTDA | 120 |
| INKRHAEIYK GLFKAELFNG KVLKQLGTVT TTEHENALLR SFDKFTTYFS GFYENRKNVF | 180 |
| SAEDISTAIP HRIVQDNFPK FKENCHIFTR LITAVPSLRE HFENVKKAIG IFVSTSIEEV | 240 |
| FSFPFYNOLL TQTQIDLYNQ LLGGISREAG TEKIKGLNEV LNLAIQKNDE TAHIIASLPH | 300 |
| RFIPLFKQIL SDRNTLSFIL EEFKSDEEVI QSFCKYKTLL RNENVLETAE ALFNELNSID | 360 |
| LTHIFISHKK LETISSALCD HWDTLRNALY ERRISELTGK ITKSAKEKVQ RSLKHEDINL | 420 |
| QEIISAAGKE LSEAFKQKTS EILSHAHAAL DOPLPTTLKK QEEKEILKSQ LDSLLGLYHL | 480 |
| LDWFAVDESN EVDPEFSARL TGIKLEMEPS LSFYNKARNY ATKKPYSVEK FKLNFQMPTL | 540 |
| ASGWDVNKEK NNGAILFVKN GLYYLGIMPK QKGRYKALSF EPTEKTSEGF DKMYYDYFPD | 600 |
| AAKMIPKCST QLKAVTAHFQ THTTPILLSN NFIEPLEITK EIYDLNNPEK EPKKFQTAYA | 660 |
| KKTGDQKGYR EALCKWIDFT RDFLSKYTKT TSIDLSSLRP SSQYKDLGEY YAELNPLLYH | 720 |
| ISFQRIAEKE IMDAVETGKL YLFQIYNKDF AKGHHGKPNL HTLYWTGLFS PENLAKTSIK | 780 |
| LNGQAELFYR PKSRMKRMAH RLGEKMLNKK LKDQKTPIPD TLYQELYDYV NHRLSHDLSD | 840 |
| EARALLPNVI TKEVSHEIIK DRRFTSDKFF FHVPITLNYQ AANSPSKFNQ RVNAYLKEHP | 900 |
| ETPIIGIDRG ERNLIYITVI DSTGKILEQR SLNTIQQFDY QKKLDNREKE RVAARQAWSV | 960 |
| VGTIKDLKQG YLSQVIHEIV DLMIHYQAVV VLENLNFGFK SKRTGIAEKA VYQQFEKMLI | 1020 |
| DKLNCLVLKD YPAEKVGGVL NPYQLTDQFT SFAKMGTQSG FLFYVPAPYT SKIDPLTGFV | 1080 |
| DPFVWKTIKN HESRKHFLEG FDFLHYDVKT GDFILHFKMN RNLSFQRGLP GFMPAWDIVF | 1140 |
| EKNETQFDAK GTPFIAGKRI VPVIENHRFT GRYRDLYPAN ELIALLEEKG IVFRDGSNIL | 1200 |
| PKLLENDDSH AIDTMVALIR SVLQMRNSNA ATGEDYINSP VRDLNGVCFD SRFQNPEWPM | 1260 |
| DADANGAYHI ALKGQLLLNH LKESKDLKLQ NGISNQDWLA YIQELRN | 1307 |
| (1090 in PCT/US2017/017255) | |
| SEQ ID NO: 29 | |
| MLKNVGIDRL DVEKGRKNMS KLEKFTNCYS LSKTLRFKAI PVGKTQENID NKRLLVEDEK | 60 |
| RAEDYKGVKK LLDRYYLSFI NDVLHSIKLK NLNNYISLFR KKTRTEKENK ELENLEINLR | 120 |
| KEIAKAFKGN EGYKSLFKKD IIETILPEFL DDKDEIALVN SFNGFTTAFT GFFDNRENMF | 180 |
| SEEAKSTSIA FRCINENLTR YISNMDIFEK VDAIFDKHEV QEIKEKILNS DYDVEDFFEG | 240 |
| EFFNFVLTQE GIDVYNAIIG GFVTESGEKI KGLNEYINLY NOKTKQKLPK FKPLYKQVLS | 300 |
| DRESLSFYGE GYTSDEEVLE VFRNTLNKNS EIFSSIKKLE KLFKNFDEYS SAGIFVKNGP | 360 |
| AISTISKDIF GEWNVIRDKW NAEYDDIHLK KKAVVTEKYE DDRRKSFKKI GSFSLEQLQE | 420 |
| YADADLSVVE KLKEIIIQKV DEIYKVYGSS EKLFDADFVL EKSLKKNDAV VAIMKDLLDS | 480 |
| VKSFENYIKA FFGEGKETNR DESFYGDFVL AYDILLKVDH IYDAIRNYVT QKPYSKDKFK | 540 |
| LYFQNPQFMG GWDKDKETDY RATILRYGSK YYLAIMDKKY AKCLQKIDKD DVNGNYEKIN | 600 |
| YKLLPGPNKM LPKVFFSKKW MAYYNPSEDI QKIYKNGTFK KGDMFNLNDC HKLIDFFKDS | 660 |
| ISRYPKWSNA YDFNFSETEK YKDIAGFYRE VEEQGYKVSF ESASKKEVDK LVEEGKLYMF | 720 |
| QIYNKDFSDK SHGTPNLHTM YFKLLFDENN HGQIRLSGGA ELFMRRASLK KEELVVHPAN | 780 |
| SPIANKNPDN PKKTTTLSYD VYKDKRFSED QYELHIPIAI NKCPKNIFKI NTEVRVLLKH | 840 |
| DDNPYVIGID RGERNLLYIV VVDGKGNIVE QYSLNEIINN FNGIRIKTDY HSLLDKKEKE | 900 |
| RFEARQNWTS IENIKELKAG YISQVVHKIC ELVEKYDAVI ALEDLNSGFK NSRVKVEKQV | 960 |
| YQKFEKMLID KLNYMVDKKS NPCATGGALK GYQITNKFES FKSMSTQNGF IFYIPAWLTS | 1020 |
| KIDPSTGFVN LLKTKYTSIA DSKKFISSFD RIMYVPEEDL FEFALDYKNF SRTDADYIKK | 1080 |
| WKLYSYGNRI RIFRNPKKNN VFDWEEVCLT SAYKELFNKY GINYQQGDIR ALLCEQSDKA | 1140 |
| FYSSFMALMS LMLQMRNSIT GRTDVDFLIS PVKNSDGIFY DSRNYEAQEN AILPKNADAN | 1200 |
| GAYNIARKVL WAIGQFKKAE DEKLDKVKIA ISNKEWLEYA QTSVKH | 1246 |
| (1091 in PCT/US2017/017255) | |
| Porphyromonas macacae | |
| SEQ ID NO: 30 | |
| MKTQHFFEDF TSLYSLSKTI RFELKPIGKT LENIKKNGLI RRDEQRLDDY EKLKKVIDEY | 60 |
| HEDFIANILS SFSFSEEILQ SYIQNLSESE ARAKIEKTMR DTLAKAFSED ERYKSIFKKE | 120 |
| LVKKDIPVWC PAYKSLCKKF DNFTTSLVPF HENRKNLYTS NEITASIPYR IVHVNLPKFI | 180 |
| QNIEALCELQ KKMGADLYLE MMENLRNVWP SFVKTPDDLC NLKTYNHLMV QSSISEYNRF | 240 |
| VGGYSTEDGT KHQGINEWIN IYRORNKEMR LPGLVFLHKQ ILAKVDSSSF ISDTLENDDQ | 300 |
| VFCVLRQFRK LFWNTVSSKE DDAASLKDLF CGLSGYDPEA IYVSDAHLAT ISKNIFDRWN | 360 |
| YISDAIRRKT EVLMPRKKES VERYAEKISK QIKKRQSYSL AELDDLLAHY SEESLPAGFS | 420 |
| LLSYFTSLGG QKYLVSDGEV ILYEEGSNIW DEVLIAFRDL QVILDKDFTE KKLGKDEEAV | 480 |
| SVIKKALDSA LRLRKFFDLL SGTGAEIRRD SSFYALYTDR MDKLKGLLKM YDKVRNYLTK | 540 |
| KPYSIEKFKL HFDNPSLLSG WDKNKELNNL SVIFRONGYY YLGIMTPKGK NLFKTLPKLG | 600 |
| AEEMFYEKME YKQIAEPMLM LPKVFFPKKT KPAFAPDOSV VDIYNKKTFK TGQKGFNKKD | 660 |
| LYRLIDFYKE ALTVHEWKLF NFSFSPTEQY RNIGEFFDEV REQAYKVSMV NVPASYIDEA | 720 |
| VENGKLYLFQ IYNKDFSPYS KGIPNLHTLY WKALFSEQNQ SRVYKLCGGG ELFYRKASLH | 780 |
| MQDTTVHPKG ISIHKKNLNK KGETSLFNYD LVKDKRFTED KFFFHVPISI NYKNKKITNV | 840 |
| NOMVRDYIAQ NDDLQIIGID RGERNLLYIS RIDTRGNLLE QFSLNVIESD KGDLRTDYQK | 900 |
| ILGDREQERL RRRQEWKSIE SIKDLKDGYM SQVVHKICNM VVEHKAIVVL ENLNLSFMKG | 960 |
| RKKVEKSVYE KFERMLVDKL NYLVVDKKNL SNEPGGLYAA YQLTNPLFSF EELHRYPQSG | 1020 |
| ILFFVDPWNT SLTDPSTGFV NLLGRINYTN VGDARKFFDR FNAIRYDGKG NILFDLDLSR | 1080 |
| FDVRVETQRK LWTLTTFGSR IAKSKKSGKW MVERIENLSL CFLELFEQFN IGYRVEKDLK | 1140 |
| KAILSQDRKE FYVRLIYLEN LMMQIRNSDG EEDYILSPAL NEKNLQFDSR LIEAKDLPVD | 1200 |
| ADANGAYNVA RKGLMVVQRI KRGDHESIHR IGRAQWLRYV QEGIVE | 1246 |
| (1092 in PCT/US2017/017255) | |
| Prevotella disiens | |
| SEQ ID NO: 31 | |
| MKVMENYQEF TNLFQLNKTL RFELKPIGKT CELLEEGKIF ASGSFLEKDK VRADNVSYVK | 60 |
| KEIDKKHKIF IEETLSSFSI SNDLLKQYFD CYNELKAFKK DCKSDEEEVK KTALRNKCTS | 120 |
| IQRAMREAIS QAFLKSPQKK LLAIKNLIEN VFKADENVQH FSEFTSYFSG FETNRENFYS | 180 |
| DEEKSTSIAY RLVHDNLPIF IKNIYIFEKL KEQFDAKTLS EIFENYKLYV AGSSLDEVFS | 240 |
| LEYFNNTLTQ KGIDNYNAVI GKIVKEDKOE IQGLNEHINL YNQKHKDRRL PFFISLKKQI | 300 |
| LSDREALSWL PDMFKNDSEV IKALKGFYIE DGFENNVLTP LATLLSSLDK YNLNGIFIRN | 360 |
| NEALSSLSQN VYRNFSIDEA IDANAELQTF NNYELIANAL RAKIKKETKO GRKSFEKYEE | 420 |
| YIDKKVKAID SLSIQEINEL VENYVSEFNS NSGNMPRKVE DYFSLMRKGD FGSNDLIENI | 480 |
| KTKLSAAEKL LGTKYQETAK DIFKKDENSK LIKELLDATK QFOHFIKPLL GTGEEADRDL | 540 |
| VFYGDFLPLY EKFEELTLLY NKVRNRLTQK PYSKDKIRLC FNKPKLMTGW VDSKTEKSDN | 600 |
| GTQYGGYLFR KKNEIGEYDY FLGISSKAQL FRKNEAVIGD YERLDYYQPK ANTIYGSAYE | 660 |
| GENSYKEDKK RLNKVIIAYI EQIKQTNIKK SIIESISKYP NISDDDKVTP SSLLEKIKKV | 720 |
| SIDSYNGILS FKSFQSVNKE VIDNLLKTIS PLKNKAEFLD LINKDYQIFT EVQAVIDEIC | 780 |
| KOKTFIYFPI SNVELEKEMG DKDKPLCLFQ ISNKDLSFAK TFSANLRKKR GAENLHTMLF | 840 |
| KALMEGNODN LDLGSGAIFY RAKSLDGNKP THPANEAIKC RNVANKDKVS LFTYDIYKNR | 900 |
| RYMENKFLFH LSIVQNYKAA NDSAQLNSSA TEYIRKADDL HIIGIDRGER NLLYYSVIDM | 960 |
| KGNIVEQDSL NIIRNNDLET DYHDLLDKRE KERKANRONW EAVEGIKDLK KGYLSQAVHQ | 1020 |
| IAQLMLKYNA IIALEDLGOM FVTRGQKIEK AVYQQFEKSL VDKLSYLVDK KRPYNELGGI | 1080 |
| LKAYQLASSI TKNNSDKQNG FLFYVPAWNT SKIDPVTGFT DLLRPKAMTI KEAQDFFGAF | 1140 |
| DNISYNDKGY FEFETNYDKF KIRMKSAQTR WTICTFGNRI KRKKDKNYWN YEEVELTEEF | 1200 |
| KKLFKDSNID YENCNLKEEI QNKDNRKFFD DLIKLLQLTL QMRNSDDKGN DYIISPVANA | 1260 |
| EGQFFDSRNG DKKLPLDADA NGAYNIARKG LWNIRQIKQT KNDKKLNLSI SSTEWLDFVR | 1320 |
| EKPYLK | 1326 |
| (1112 in PCT/US2017/017255) | |
| Alicyclobacillus acidoterrestris | |
| SEQ ID NO: 32 | |
| MAVKSIKVKL RLDDMPEIRA GLWKLHKEVN AGVRYYTEWL SLLRQENLYR RSPNGDGEQE | 60 |
| CDKTAEECKA ELLERLRARQ VENGHRGPAG SDDELLQLAR QLYELLVPQA IGAKGDAQQI | 120 |
| ARKFLSPLAD KDAVGGLGIA KAGNKPRWVR MREAGEPGWE EEKEKAETRK SADRTADVLR | 180 |
| ALADFGLKPL MRVYTDSEMS SVEWKPLRKG QAVRTWDRDM FQQAIERMMS WESWNQRVGQ | 240 |
| EYAKLVEQKN RFEQKNFVGQ EHLVHLVNQL QQDMKEASPG LESKEQTAHY VTGRALRGSD | 300 |
| KVFEKWGKLA PDAPFDLYDA EIKNVORRNT RRFGSHDLFA KLAEPEYQAL WREDASFLTR | 360 |
| YAVYNSILRK LNHAKMFATF TLPDATAHPI WTRFDKLGGN LHQYTFLFNE FGERRHAIRF | 420 |
| HKLLKVENGV AREVDDVTVP ISMSEQLDNL LPRDPNEPIA LYFRDYGAEQ HFTGEFGGAK | 480 |
| IQCRRDQLAH MHRRRGARDV YLNVSVRVQS QSEARGERRP PYAAVFRLVG DNHRAFVHFD | 540 |
| KLSDYLAEHP DDGKLGSEGL LSGLRVMSVD LGLRTSASIS VFRVARKDEL KPNSKGRVPF | 600 |
| FFPIKGNDNL VAVHERSQLL KLPGETESKD LRAIREERQR TLRQLRTQLA YLRLLVRCGS | 660 |
| EDVGRRERSW AKLIEQPVDA ANHMTPDWRE AFENELQKLK SLHGICSDKE WMDAVYESVR | 720 |
| RVWRHMGKQV RDWRKDVRSG ERPKIRGYAK DVVGGNSIEQ IEYLERQYKF LKSWSFFGKV | 780 |
| SGQVIRAEKG SRFAITLREH IDHAKEDRLK KLADRIIMEA LGYVYALDER GKGKWVAKYP | 840 |
| PCQLILLEEL SEYQFNNDRP PSENNQLMQW SHRGVFQELI NQAQVHDLLV GTMYAAFSSR | 900 |
| FDARTGAPGI RCRRVPARCT QEHNPEPFPW WLNKFVVEHT LDACPLRADD LIPTGEGEIF | 960 |
| VSPFSAEEGD FHQIHADLNA AQNLQQRLWS DFDISQIRLR CDWGEVDGEL VLIPRLTGKR | 1020 |
| TADSYSNKVF YTNTGVTYYE RERGKKRRKV FAQEKLSEEE AELLVEADEA REKSVVLMRD | 1080 |
| PSGIINRGNW TRQKEFWSMV NORIEGYLVK QIRSRVPLQD SACENTGDI | 1129 |
| (1113 in PCT/US2017/017255) | |
| Alicyclobacillus contaminans | |
| SEQ ID NO: 33 | |
| MGFNTAELLR KVEEEMRKTS VGFDTDNPFA HRITRRAIRG WDRIAEAWRR LPPDAPESEY | 60 |
| IEAFKDIQRK NPRKIGSEPL FKNLAAPGVR SELLNNPQVL ITFAKYNELQ RQLAKAKQFA | 120 |
| QKTLPHPVFH PVWVRYDKLG GNLHHYQIEP AVHANDTHKV KFSSLLLPQE DGSYAEVKDV | 180 |
| TVSLAPSLOF PTGLVHPKVT TPPRTGLVTV MDEEAGKPVV CYRDRGHDAL VPVAFGGAKL | 240 |
| QFNRAHLSAG YRKGVLSAGG GGSIYFNVTL DVQVPNERDV SKTFSFSRDR DLVSLKAEEL | 300 |
| KRYMETKPLG MPGVRVMSVD LGVRYGAAIS VFEVKPFAEV RKDKLHYPIT GCEGFVAEHE | 360 |
| RSVILKLPGE GVRTAGKQSE RKQALAAIRA EMSILRKWLR VSQVTEEDRA KAVRGLLEDE | 420 |
| RGGGWTMDPG EDSDHOPLQQ FLHEARLAVG ELVNLVHLSP AEWERAVIER HRRLERITAS | 480 |
| HIRVFQTMRK VWGKRRNEDA AHTGGISLAH IEHLIQQRKL FIRWSTHART YGEVRRLPKH | 540 |
| EGFAKRLQKH TNHVKEDRIK KLADMIVMAA RGYRFLDKRA RWVKTRHAPC DLILFEDLSR | 600 |
| YRFTMDRPPT ENSQLMNWSH RELLKTVKMQ AALFGIGVGT VPAAFTSRFD AQTGAPGLRC | 660 |
| KRVTKQDKEK TPFWLIQFAE ITGVNVTNVE PGQLIPVDGG EWFVSPKGPR AADGLKCVHA | 720 |
| DINAAHNLQR RFWIPRLPSV KCRRYVEAEG FAAVPSSTAF MKVHGKGAFV SVDGEFYEYQ | 780 |
| KGRRVAVNRA DRTSSTLDED EGDIGEEMLV SSNGAGEFVR MFYDESGYVG YGRWMDSKVF | 840 |
| WGKVRQIVHR AIQDQVEKRA AARGENGATS SR | 872 |
| (1114 in PCT/US2017/017255) | |
| Desulfovibrio inopinatus | |
| SEQ ID NO: 34 | |
| MPTRTINLKL VLGKNPENAT LRRALFSTHR LVNQATKRIE EFLLLCRGEA YRTVDNEGKE | 60 |
| AEIPRHAVQE EALAFAKAAQ RHNGCISTYE DQEILDVLRQ LYERLVPSVN ENNEAGDAçA | 120 |
| ANAWVSPLMS AESEGGLSVY DKVLDPPPVW MKLKEEKAPG WEAASQIWIQ SDEGQSLLNK | 180 |
| PGSPPRWIRK LRSGQPWQDD FVSDQKKKQD ELTKGNAPLI KOLKEMGLLP LVNPFFRHLL | 240 |
| DPEGKGVSPW DRLAVRAAVA HFISWESWNH RTRAEYNSLK LRRDEFEAAS DEFKDDFTLL | 300 |
| RQYEAKRHST LKSIALADDS NPYRIGVRSL RAWNRVREEW IDKGATEEQR VTILSKLQTQ | 360 |
| LRGKFGDPDL FNWLAQDRHV HLWSPRDSVT PLVRINAVDK VLRRRKPYAL MTFAHPRFHP | 420 |
| RWILYEAPGG SNLROYALDC TENALHITLP LLVDDAHGTW IEKKIRVPLA PSGQIQDLTL | 480 |
| EKLEKKKNRL YYRSGFQQFA GLAGGAEVLF HRPYMEHDER SEESLLERPG AVWFKLTLDV | 540 |
| ATQAPPNWLD GKGRVRTPPE VHHFKTALSN KSKHTRTLQP GLRVLSVDLG MRTFASCSVF | 600 |
| ELIEGKPETG RAFPVADERS MDSPNKLWAK HERSFKLTLP GETPSRKEEE ERSIARAEIY | 660 |
| ALKRDIQRLK SLLRLGEEDN DNRRDALLEQ FFKGWGEEDV VPGQAFPRSL FOGLGAAPFR | 720 |
| STPELWROHC QTYYDKAEAC LAKHISDWRK RTRPRPTSRE MWYKTRSYHG GKSIWMLEYL | 780 |
| DAVRKLLLSW SLRGRTYGAI NRODTARFGS LASRLLHHIN SLKEDRIKTG ADSIVQAARG | 840 |
| YIPLPHGKGW EQRYEPCQLI LFEDLARYRF RVDRPRRENS QLMQWNHRAI VAETTMQAEL | 900 |
| YGQIVENTAA GFSSRFHAAT GAPGVRCRFL LERDFDNDLP KPYLLRELSW MLGNTKVESE | 960 |
| EEKLRLLSEK IRPGSLVPWD GGEQFATLHP KRQTLCVIHA DMNAAQNLQR RFFGRCGEAF | 1020 |
| RLVCQPHGDD VLRLASTPGA RLLGALQQLE NGQGAFELVR DMGSTSQMNR FVMKSLGKKK | 1080 |
| IKPLQDNNGD DELEDVLSVL PEEDDTGRIT VFRDSSGIFF PCNVWIPAKQ FWPAVRAMIW | 1140 |
| KVMASHSLG | 1149 |
| (1115 in PCT/US2017/017255) | |
| Desulfonatronum thiodismutans | |
| SEQ ID NO: 35 | |
| MVLGRKDDTA ELRRALWTTH EHVNLAVAEV ERVLLRCRGR SYWTLDRRGD PVHVPESQVA | 60 |
| EDALAMAREA QRRNGWPVVG EDEEILLALR YLYEQIVPSC LLDDLGKPLK GDAQKIGTNY | 120 |
| AGPLFDSDTC RRDEGKDVAC CGPFHEVAGK YLGALPEWAT PISKQEFDGK DASHLRFKAT | 180 |
| GGDDAFFRVS IEKANAWYED PANQDALKNK AYNKDDWKKE KDKGISSWAV KYIQKQLQLG | 240 |
| QDPRTEVRRK LWLELGLLPL FIPVFDKTMV GNLWNRLAVR LALAHLLSWE SWNHRAVQDQ | 300 |
| ALARAKRDEL AALFLGMEDG FAGLREYELR RNESIKQHAF EPVDRPYVVS GRALRSWTRV | 360 |
| REEWLRHGDT QESRKNICNR LQDRLRGKFG DPDVFHWLAE DGQEALWKER DCVTSFSLLN | 420 |
| DADGLLEKRK GYALMTFADA RLHPRWAMYE APGGSNLRTY QIRKTENGLW ADVVLLSPRN | 480 |
| ESAAVEEKTF NVRLAPSGOL SNVSFDQIQK GSKMVGRCRY QSANQQFEGL LGGAEILFDR | 540 |
| KRIANEQHGA TDLASKPGHV WFKLTLDVRP QAPQGWLDGK GRPALPPEAK HFKTALSNKS | 600 |
| KFADQVRPGL RVLSVDLGVR SFAACSVFEL VRGGPDQGTY FPAADGRTVD DPEKLWAKHE | 660 |
| RSFKITLPGE NPSRKEEIAR RAAMEELRSL NGDIRRLKAI LRLSVLQEDD PRTEHLRLFM | 720 |
| EAIVDDPAKS ALNAELFKGF GDDRFRSTPD LWKQHCHFFH DKAEKVVAER FSRWRTETRP | 780 |
| KSSSWQDWRE RRGYAGGKSY WAVTYLEAVR GLILRWNMRG RTYGEVNRQD KKQFGTVASA | 840 |
| LLHHINQLKE DRIKTGADMI IQAARGFVPR KNGAGWVQVH EPCRLILFED LARYRFRTDR | 900 |
| SRRENSRLMR WSHREIVNEV GMQGELYGLH VDTTEAGFSS RYLASSGAPG VRCRHLVEED | 960 |
| FHDGLPGMHL VGELDWLLPK DKDRTANEAR RLLGGMVRPG MLVPWDGGEL FATLNAASQL | 1020 |
| HVIHADINAA QNLORREWGR CGEAIRIVCN QLSVDGSTRY EMAKAPKARL LGALQQLKNG | 1080 |
| DAPFHLTSIP NSQKPENSYV MTPTNAGKKY RAGPGEKSSG EEDELALDIV EQAEELAQGR | 1140 |
| KTFFRDPSGV FFAPDRWLPS EIYWSRIRRR IWQVTLERNS SGRQERAEMD EMPY | 1194 |
| (1116 in PCT/US2017/017255) | |
| Tuberibacillus calidus | |
| SEQ ID NO: 36 | |
| MATKSFILKM KTKNNPQLRL SLWKTHELFN FGVAYYMDLL SLFRQKDLYM HNDEDPDHPV | 60 |
| VLKKEEIQER LWMKVRETQQ KNGFHGEVSK DEVLETLRAL YEELVPSAVG KSGEANQISN | 120 |
| KYLYPLTDPA SQSGKGTANS GRKPRWKKLK EAGDPSWKDA YEKWEKERQE DPKLKILAAL | 180 |
| QSFGLIPLFR PFTENDHKAV ISVKWMPKSK NQSVRKFDKD MENQAIERFL SWESWNEKVA | 240 |
| EDYEKTVSIY ESLQKELKGI STKAFEIMER VEKAYEAHLR EITFSNSTYR IGNRAIRGWT | 300 |
| EIVKKWMKLD PSAPQGNYLD VVKDYQRRHP RESGDFKLFE LLSRPENQAA WREYPEFLPL | 360 |
| YVKYRHAEQR MKTAKKOATF TLCDPIRHPL WVRYEERSGT NLNKYRLIMN EKEKVVQFDR | 420 |
| LICLNADGHY EEQEDVTVPL APSQQFDDQI KFSSEDTGKG KHNFSYYHKG INYELKGTLG | 480 |
| GARIQFDREH LLRROGVKAG NVGRIFLNVT LNIEPMQPFS RSGNLQTSVG KALKVYVDGY | 540 |
| PKVVNFKPKE LTEHIKESEK NTLTLGVESL PTGLRVMSVD LGQRQAAAIS IFEVVSEKPD | 600 |
| DNKLFYPVKD TDLFAVHRTS FNIKLPGEKR TERRMLEQQK RDQAIRDLSR KLKFLKNVLN | 660 |
| MQKLEKTDER EKRVNRWIKD REREEENPVY VQEFEMISKV LYSPHSVWVD QLKSIHRKLE | 720 |
| EQLGKEISKW RQSISQGRQG VYGISLKNIE DIEKTRRLLF RWSMRPENPG EVKQLQPGER | 780 |
| FAIDQQNHLN HLKDDRIKKL ANQIVMTALG YRYDGKRKKW IAKHPACQLV LFEDLSRYAF | 840 |
| YDERSRLENR NLMRWSRREI PKOVAQIGGL YGLLVGEVGA QYSSRFHAKS GAPGIRCRVV | 900 |
| KEHELYITEG GQKVRNOKFL DSLVENNIIE PDDARRLEPG DLIRDOGGDK FATLDERGEL | 960 |
| VITHADINAA QNLQKREWTR THGLYRIRCE SREIKDAVVL VPSDKDQKEK MENLFGIGYL | 1020 |
| QPFKQENDVY KWVKGEKIKG KKTSSQSDDK ELVSEILQEA SVMADELKGN RKTLFRDPSG | 1080 |
| YVFPKDRWYT GGRYFGTLEH LLKRKLAERR LFDGGSSRRG LENGTDSNTN VE | 1132 |
| (1117 in PCT/US2017/017255) | |
| Bacillus thermoamylovorans | |
| SEQ ID NO: 37 | |
| MATRSFILKI EPNEEVKKGL WKTHEVLNHG IAYYMNILKL IRQEAIYEHH EQDPKNPKKV | 60 |
| SKAEIQAELW DFVLKMQKCN SFTHEVDKDV VFNILRELYE ELVPSSVEKK GEANQLSNKF | 120 |
| LYPLVDPNSQ SGKGTASSGR KPRWYNIKIA GDPSWEEEKK KWEEDKKKDP LAKILGKLAE | 180 |
| YGLIPLFIPF TDSNEPIVKE IKWMEKSRNQ SVRRLDKDMF IQALERFLSW ESWNLKVKEE | 240 |
| YEKVEKEHKT LEERIKEDIQ AFKSLEQYEK ERQEQLLRDT LNTNEYRLSK RGLRGWREII | 300 |
| QKWLKMDENE PSEKYLEVFK DYQRKHPREA GDYSVYEFLS KKENHFIWRN HPEYPYLYAT | 360 |
| FCEIDKKKKD AKQQATFTLA DPINHPLWVR FEERSGSNLN KYRILTEQLH TEKLKKKLTV | 420 |
| QLDRLIYPTE SGGWEEKGKV DIVLLPSRQF YNQIFLDIEE KGKHAFTYKD ESIKFPLKGT | 480 |
| LGGARVQFDR DHLRRYPHKV ESGNVGRIYF NMTVNIEPTE SPVSKSLKIH RDDFPKFVNF | 540 |
| KPKELTEWIK DSKGKKLKSG IESLEIGLRV MSIDLGQRQA AAASIFEVVD QKPDIEGKLF | 600 |
| FPIKGTELYA VHRASFNIKL PGETLVKSRE VLRKAREDNL KLMNQKLNFL RNVLHFQQFE | 660 |
| DITEREKRVT KWISRQENSD VPLVYQDELI QIRELMYKPY KDWVAFLKQL HKRLEVEIGK | 720 |
| EVKHWRKSLS DGRKGLYGIS LKNIDEIDRT RKFLLRWSLR PTEPGEVRRL EPGQRFAIDQ | 780 |
| LNHLNALKED RLKKMANTII MHALGYCYDV RKKKWQAKNP ACQIILFEDL SNYNPYEERS | 840 |
| RFENSKLMKW SRREIPROVA LQGEIYGLQV GEVGAQFSSR FHAKTGSPGI RCSVVTKEKL | 900 |
| QDNRFFKNLQ REGRLTLDKI AVLKEGDLYP DKGGEKFISL SKDRKLVTTH ADINAAQNLQ | 960 |
| KREWTRTHGF YKVYCKAYQV DGQTVYIPES KDQKQKIIEE FGEGYFILKD GVYEWGNAGK | 1020 |
| LKIKKGSSKQ SSSELVDSDI LKDSFDLASE LKGEKLMLYR DPSGNVFPSD KWMAAGVFFG | 1080 |
| KLERILISKL TNQYSISTIE DDSSKQSM | 1108 |
| (1118 in PCT/US2017/017255) | |
| SEQ ID NO: 38 | |
| MAIRSIKLKL KTHTGPEAQN LRKGIWRTHR LLNEGVAYYM KMLLLFRQES TGERPKEELQ | 60 |
| EELICHIREQ QQRNQADKNT QALPLDKALE ALROLYELLV PSSVGQSGDA QIISRKFLSP | 120 |
| LVDPNSEGGK GTSKAGAKPT WQKKKEANDP TWEQDYEKWK KRREEDPTAS VITTLEEYGI | 180 |
| RPIFPLYTNT VTDIAWLPLQ SNQFVRTWDR DMLQQAIERL LSWESWNKRV QEEYAKLKEK | 240 |
| MAQLNEQLEG GQEWISLLEQ YEENRERELR ENMTAANDKY RITKROMKGW NELYELWSTF | 300 |
| PASASHEQYK EALKRVQQRL RGRFGDAHFF QYLMEEKNRL IWKGNPQRIH YFVARNELTK | 360 |
| RLEEAKQSAT MTLPNARKHP LWVRFDARGG NLQDYYLTAE ADKPRSRREV TFSQLIWPSE | 420 |
| SGWMEKKDVE VELALSROFY QQVKLLKNDK GKQKIEFKDK GSGSTFNGHL GGAKLQLERG | 480 |
| DLEKEEKNFE DGEIGSVYLN VVIDFEPLQE VKNGRVQAPY GQVLQLIRRP NEFPKVTTYK | 540 |
| SEQLVEWIKA SPOHSAGVES LASGFRVMSI DLGLRAAAAT SIFSVEESSD KNAADFSYWI | 600 |
| EGTPLVAVHQ RSYMLRLPGE QVEKQVMEKR DERFOLHQRV KFQIRVLAQI MRMANKQYGD | 660 |
| RWDELDSLKQ AVEQKKSPLD QTDRTFWEGI VCDLTKVLPR NEADWEQAVV QIHRKAEEYV | 720 |
| GKAVQAWRKR FAADERKGIA GLSMWNIEEL EGLRKLLISW SRRTRNPQEV NRFERGHTSH | 780 |
| QRLLTHIQNV KEDRLKOLSH AIVMTALGYV YDERKQEWCA EYPACQVILF ENLSQYRSNL | 840 |
| DRSTKENSTL MKWAHRSIPK YVHMQAEPYG IQIGDVRAEY SSRFYAKTGT PGIRCKKVRG | 900 |
| QDLQGRRFEN LQKRLVNEQF LTEEQVKQLR PGDIVPDDSG ELFMTLTDGS GSKEVVFLQA | 960 |
| DINAAHNLQK RFWQRYNELF KVSCRVIVRD EEEYLVPKTK SVQAKLGKGL FVKKSDTAWK | 1020 |
| DVYVWDSQAK LKGKTTFTEE SESPEQLEDF QEIIEEAEEA KGTYRTLFRD PSGVFFPESV | 1080 |
| WYPQKDFWGE VKRKLYGKLR ERFLTKAR | 1108 |
| (1119 in PCT/US2017/017255) | |
| Methylobacterium nodulans | |
| SEQ ID NO: 39 | |
| MLTKQDKQQK ITYCTNMNEV FEAKLGSADL LLNWDHLRGR IRDRVDAGDI GSAFLKLALD | 60 |
| VAHVLPDGVD DOLARAAFHF QSAKGAKSKH ADSVQAGLRV LSIDLGVRSF ATCSVFELKD | 120 |
| TAPTTGVAFP LAEFRLWAVH ERSFTLELPG ENVGAAGQQW RAQADAELRO LRGGLNRHRQ | 180 |
| LLRAATVOKG ERDAYLTDLR EAWSAKELWP FEASLLSELE RCSTVADPLW QDTCKRAARL | 240 |
| YRTEFGAVVS EWRSRTRSRE DRKYAGKSMW SVQHLTDVRR FLQSWSLAGR ASGDIRRLDR | 300 |
| ERGGVFAKDL LDHIDALKDD RLKTGADLIV QAARGFORNE FGYWVQKHAP CHVILFEDLS | 360 |
| RYRMRTDRPR RENSQLMOWA HRGVPDMVGM QGEIYGIQDR RDPDSARKHA RQPLAAFCLD | 420 |
| TPAAFSSRYH ASTMTPGIRC HPLRKREFED QGFLELLKRE NEGLDLNGYK PGDLVPLPGG | 480 |
| EVFVCLNANG LSRIHADINA AQNLQRRFWT QHGDAFRLPC GKSAVQGQIR WAPLSMGKRQ | 540 |
| AGALGGFGYL EPTGHDSGSC QWRKTTEAEW RRLSGAQKDR DEAAAAEDEE LQGLEEELLE | 600 |
| RSGERVVFFR DPSGVVLPTD LWFPSAAFWS IVRAKTVGRL RSHLDAQAEA SYAVAAGL | 658 |
| (1120 in PCT/US2017/017255) | |
| SEQ ID NO: 40 | |
| MKKFELKQNF RNNYSGKTLR NFRQTLAQIA NKKSSDSILT IKFKLDCSKT GKLPKYENLI | 60 |
| SLYDTIEDIK KGTLSYYLFT LIVSGFKFFG SASQAKAFST KDIFKDNDFY NQFKIQSHLD | 120 |
| LPDFVPSKIY QRLKKNVRST NGKDNAFKAS VIVAEYRKEI GKLKNKDESS EHQCEELFKK | 180 |
| IGTALETRES SWQDLINNCS TGCEIIDEIL NDSFGTLPSI KKMVLASTTQ SSDGEQDGIA | 240 |
| IAYDPDSTFI KSDELLNPYF AVATILKSMP PEIQQDKKSA YVKANLTTPT HNALSWIFGK | 300 |
| GLTLFQTEST EKLCAMFNVS DKRVIEQVQD AAKAVKLPAE LDLNHCTLKF QDFRSSLGGH | 360 |
| LDSWTTNYLK RLDELNDLLL NLPKNLSLPD IFMIDGKDFI EYSGCNRDEI QQMIDFVVNE | 420 |
| QNRIKLQESL NALLGKGNNQ ICSDDISTVK DFSEIVNSLH SFVQQIDNSL EQSSNEANSI | 480 |
| FSELKKKIEK NEKWDIWKNN LKKIPKLNKL SGGVPDAWKE IREIEQKFHE ISENQKKHFT | 540 |
| EVMEWIDAGN GTIDIFESRF KYDELLKKSK KNNLQSADEL AFRSVLNKLG RFARQGNDLV | 600 |
| CEKIKNWFKE QNIFDSSKDF NRYFINQKGF IFKHPSSKKD NSPYNLSANL LEKRYEVTNT | 660 |
| VGALLEQCES DPAIVNDPFS MRSLVEFRAL WFSINISGIS KEQHIPTKIA QPKLDDSTYQ | 720 |
| ESVSPTLKYR LEKEQITSSE LNSIFTVYKS LLSGLSIRLS RNSFYLRTKF SWIGNNSLIY | 780 |
| CPKETTWKIP AAYFKSDLWN EYKDKQILIV NEEYDVDVVK TFESVYKIVK SKDNNEKNRI | 840 |
| LPLLKQLPHD WMFKLPFGAS NAEKCKVLKL EKNNKKFKPL SVSKDSLARL SGPSTYFNQI | 900 |
| DEIMMNDESE LSEMTLLADE PVRQQMSNGK IEIIPDDYVM SLAIPITRSL KKGNTESFPF | 960 |
| KNIVSIDQGE AGFAYAVFKL SDCGNERAEP IATGLIPIPS IRRLIHSVKK YRGKKQRIQN | 1020 |
| FNQKFDSTMF TLRENVTGDI CGLIVALMKK YNAFPILEKQ VGNLESGSKQ LMLVYKAVNS | 1080 |
| KFLAAKVDMQ NDQRRSWWYQ GNSWNTPILR ISNPNQSNNK NIVKNINGKK YEELKIYPGY | 1140 |
| SVSAYMTSCI CHVCGRNALE LLKNDDSTGK VKKYQINQDG EVTIGGEVIK LYRKPDRLTP | 1200 |
| VKNLAKKGNR ERTYASINER APTVSVQKAE LSADELQKII KKNMRRAPRS LMSKDTTQSR | 1260 |
| YFCVFKNCPC HNKEQHADVN AAINIGRRFL KDCILDDNKE KD | 1302 |
| (1121 in PCT/US2017/017255) | |
| SEQ ID NO: 41 | |
| MRSNYHGGRN ARQWRKQISG LARRTKETVF TYKFPLETDA AEIDFDKAVQ TYGIAEGVGH | 60 |
| GSLIGLVCAF HLSGFRLFSK AGEAMAFRNR SRYPTDAFAE KLSAIMGIQL PTLSPEGLDL | 120 |
| IFQSPPRSRD GIAPVWSENE VRNRLYTNWT GRGPANKPDE HLLEIAGEIA KQVFPKFGGW | 180 |
| DDLASDPDKA LAAADKYFQS QGDFPSIASL PAAIMLSPAN STVDFEGDYI AIDPAAETLL | 240 |
| HOAVSRCAAR LGRERPDLDO NKGPFVSSLQ DALVSSONNG LSWLFGVGFQ HWKEKSPKEL | 300 |
| IDEYKVPADQ HGAVTQVKSF VDAIPLNPLF DTTHYGEFRA SVAGKVRSWV ANYWKRLLDL | 360 |
| KSLLATTEFT LPESISDPKA VSLFSGLLVD PQGLKKVADS LPARLVSAEE AIDRLMGVGI | 420 |
| PTAADIAQVE RVADEIGAFI GOVQQFNNQV KOKLENLQDA DDEEFLKGLK IELPSGDKEP | 480 |
| PAINRISGGA PDAAAEISEL EEKLORLLDA RSEHFQTISE WAEENAVTLD PIAAMVELER | 540 |
| LRLAERGATG DPEEYALRLL LORIGRLANR VSPVSAGSIR ELLKPVFMEE REFNLFFHNR | 600 |
| LGSLYRSPYS TSRHOPFSID VGKAKAIDWI AGLDQISSDI EKALSGAGEA LGDQLRDWIN | 660 |
| LAGFAISQRL RGLPDTVPNA LAQVRCPDDV RIPPLLAMLL EEDDIARDVC LKAFNLYVSA | 720 |
| INGCLFGALR EGFIVRTRFQ RIGTDQIHYV PKDKAWEYPD RLNTAKGPIN AAVSSDWIEK | 780 |
| DGAVIKPVET VRNLSSTGFA GAGVSEYLVQ APHDWYTPLD LRDVAHLVTG LPVEKNITKL | 840 |
| KRLTNRTAFR MVGASSFKTH LDSVLLSDKI KLGDFTIIID QHYRQSVTYG GKVKISYEPE | 900 |
| RLQVEAAVPV VDTRDRTVPE PDTLFDHIVA IDLGERSVGF AVFDIKSCLR TGEVKPIHDN | 960 |
| NGNPVVGTVA VPSIRRLMKA VRSHRRRRQP NOKVNOTYST ALQNYRENVI GDVCNRIDTL | 1020 |
| MERYNAFPVL EFQIKNFQAG AKOLEIVYGS VLHRYTFSGV DAHKAKRREY WYNGELWEHP | 1080 |
| YLMAKKWNEE TNSMSGAPKP VSLFPGVTVN AARTSQICHQ CORNPMSHLR GLTGTIEISS | 1140 |
| DGLLELDDGT IRLFETSDYD EDKFKQSRRE KRRLDANVLL SGRHRAEYIY TVAKRNLRRP | 1200 |
| PKNVMTKDTT QSRYTCLYKN CSWTGHADEN AAINIGRRYL AERIDMPASK TKAAV | 1255 |
| (1122 in PCT/US2017/017255) | |
| SEQ ID NO: 42 | |
| MRPRFHGGMN ARDWRKHVGV LAQQHKETTR TYTFPLDTTG SAIDFDAALQ AYNAVEGVGY | 60 |
| GSLLGLACAV HLSGFRLFST GKEAATFRNR ARYPNAAFQA ALRKELGTTI TTLTPETLDR | 120 |
| LFSSRPKRRN GVPLPWNQDS IRDRLYTNWV KPRPGDTPDA VLFQIATGIA QEITEDVSSW | 180 |
| TDLAKNSDRG LKAAHRYFAR VGGFPAFDNL TPPATVQPTD TTIDYDPNAP FHLVSHADQT | 240 |
| LIHQSISLCA HRIRQEDPAL DPNKSGFIKQ LONNFLSQTF YGLSWLFGAG YVHFRECTAN | 300 |
| DLAIQYGIPN NCRDGIHQIK SFADAILPNT FFEKKHYRKD SRSVGKKAKS WISNYWQRLL | 360 |
| QLQTWVDDHT WVTLPQELTE AQFKPLFRGL LVDAVELMAI AERLPORLAD CRDSLDCLMG | 420 |
| KGPQAATKND VEIVEKVREE IESFVGQIEQ LGNQLRHQLE NENNDQVHRD NLHQLKNRLP | 480 |
| LDLRRPQALN KISGGVPDVA KSIRGLETQL DQVLKERRSH FGRLTKWAKE CGITLDPLQP | 540 |
| LIESEKORVA ERGSAHDAKE LAIRLLLQRI GRLGHRLSPT NATAIQELLR PVFAVKREFN | 600 |
| LFFHNHMGAL YRSPYSTSRH QPFQINVDVA HGTDWIGTIE TLIQNLFTQI QDDALLRDLV | 660 |
| QLEGFVFSHK LRALPGVIPS ELARPNNLQQ MGLPALLLVL LOADQVHRET VLRVFNLYGS | 720 |
| AINGYLFOAL RPGFIVRAGF QRLETKKLRY VPKAQSWQYP DRLHHAKSAI KNSLSAGWIK | 780 |
| KNHQGAILPQ KTLTALVKQK SLKDTGVPEY LVQAPHDWYV PIDLRGPAIP IEGLTVGTEG | 840 |
| PELTOLGPMK DDCAFRAIGP SSFKSKIDAG LLPQDVKYGD MTLIFDQHYQ QSISFANGTF | 900 |
| SIQYQPTSLQ VKAAIPVVDK RPRDTRNNSH LYDRIVAIDL GERKIGYAIF DLKQVLKSEQ | 960 |
| LEPMREDGKP LIGSISIRSI RGLMKAVQTH RNRROPNYRI DOTYSKALMH YRESVIGDVC | 1020 |
| NAIDTLCARY GGFPVLESSV RNFEVGSAQL KTVYGSVSRR YTWSAVDAHK NORQQYWLGG | 1080 |
| TKDKIPIWTH PYLMTREWDE KNSKWSNRSK PLKMHPGVEV HPAGTSQICH QCKRNPIGAL | 1140 |
| WNVADTVVLD DOGQLDLDDG TIRLNSGYID TTEIKRARRK KIRLPENKPL TGSHKTSHVR | 1200 |
| AVARRNLRQP PKSTRAKDTT QSRYTCLYVD CGHECHADEN AAINIGRKYL QERIHIEASR | 1260 |
| QALSTR | 1266 |
| (1123 in PCT/US2017/017255) | |
| SEQ ID NO: 43 | |
| MVAGLKKIKR DGVTMKSNYH GGVKARAWRK RIGGLARROK ETVFTYKFPL ETEEAGIDFD | 60 |
| KAVQTYGIAE GISQGSLIGL VCAFHLSGFR LFSKADETKA FCNQGRYPNQ AFAEKLRNEL | 120 |
| SVTLPKLSPQ SLDVLFQSSP KSKNGVAPEW SKNAIRNRLY TNWTGKGAGT NPDEHLLEIA | 180 |
| EDIAAEIDSD LDGWKDLEEH PEKGLSAADR YFQAQGDFPS LTGLPPSVPL TPQNSTVAFE | 240 |
| GDPVCLNPSD NTLLHQAVAR CAGRILQEQP NLSPDKNRFI NQLQDELVSS QNNGLSWLFG | 300 |
| VGFKYWKEMS VDQLADDYKV KSTDLDALKQ VKSFIDAIPL NPLFDTPHYG EFRASVAGKM | 360 |
| RSWVKNYWKR LLDLKSQLGT ANINLPEGLD EQRAENLFSG LLIDSKGLRQ VTDKLPSRLK | 420 |
| KAEDTIDRLM GDGNPTSDDI EQVETVAAEI SAFIGQVEQF NNQLEQRLEN PLEGDDETFL | 480 |
| KQLKIDLPAE FKKPPAINRI SGGSPDPTAE IAELEEKLDR LMSARKEHYE TIAEWASANK | 540 |
| VTLDPMEAMT TLEAQRLTER GAEGDQEEFA LRLLLQRIGR LANRLSPQGA TAIRDLLRPV | 600 |
| FTEKREFNLF FHNRMGSLYR SPYSTSRHQP FTIDVAVAKN TDWMDALDGI AETIMKGLSQ | 660 |
| AGDELSLRLR DWINISGFSL SQRLRGLPDT VPGELALVRS ADDVRIPPML ALQLEEDEVS | 720 |
| REVCLKAFNL YVSAINGCLF RALREGFIVR TKFQRLERDV LSYVPKTKLW NYPQRLDTAR | 780 |
| GPIHSALAAA WINKEGSVID PVETVTALSD TGFSDDGIPE YLVQAPHDWY TPIDLRDISK | 840 |
| PVSGLPVKKN ITGLKROKKQ TAFRMVGPSS FKSHLDSTLL SEEVKLGDFT LIFDQYYKQR | 900 |
| VSYNGRVKIT FEPDRLHVEA AVPVIDKRVR PSTEEDALFD HLLAIDLGEK RVGYAVYDIK | 960 |
| ACLRTGDIKP LEDGDGKPIV GSVAVPSIRR LMKAVRSHRQ QRQPNQKVNQ TYSTALMNYR | 1020 |
| ENVIGDVCNR IDTLMEKYNA FPVLESSVMN FEAGSRQLEM VYGSVLHRYT YSKIDAHTAK | 1080 |
| RKEYWYTGEY WDHPYLMAHK WNERTRSYSG SLSALTLYPG VMVHPAGTSQ RCHQCKRNPM | 1140 |
| VEIKOLTGQV EINADGSLEL DDGTICLYEG YDYSPEEYKK AKREKRRLDP NVPLSGRHQA | 1200 |
| KHVSAVAKRN LRRPTVSMMS GDTTQARYVC LYTDCDFTGH ADENAAINIG WKYLTERIAL | 1260 |
| SESKDKAGV | 1269 |
| (1124 in PCT/US2017/017255) | |
| SEQ ID NO: 44 | |
| MQISKVNHKH VAVGQKDRER ITGFIYNDPV GDEKSLEDVV AKRANDTKVL FNVFNTKDLY | 60 |
| DSQESDKSEK DKEIISKGAK FVAKSFNSAI TILKKONKIY STLTSQQVIK ELKDKFGGAR | 120 |
| IYDDDIEEAL TETLKKSFRK ENVRNSIKVL IENAAGIRSS LSKDEEELIQ EYFVKQLVEE | 180 |
| YTKTKLQKNV VKSIKNONMV IQPDSDSQVL SLSESRREKQ SSAVSSDTLV NCKEKDVLKA | 240 |
| FLTDYAVLDE DERNSLLWKL RNLVNLYFYG SESIRDYSYT KEKSVWKEHD EQKANKTLFI | 300 |
| DEICHITKIG KNGKEQKVLD YEENRSRCRK QNINYYRSAL NYAKNNTSGI FENEDSNHFW | 360 |
| IHLIENEVER LYNGIENGEE FKFETGYISE KVWKAVINHL SIKYIALGKA VYNYAMKELS | 420 |
| SPGDIEPGKI DDSYINGITS FDYEIIKAEE SLORDISMNV VFATNYLACA TVDTDKDFLL | 480 |
| FSKEDIRSCT KKDGNLCKNI MQFWGGYSTW KNFCEEYLKD DKDALELLYS LKSMLYSMRN | 540 |
| SSFHFSTENV DNGSWDTELI GKLFEEDCNR AARIEKEKFY NNNLHMFYSS SLLEKVLERL | 600 |
| YSSHHERASQ VPSFNRVFVR KNFPSSLSEQ RITPKFTDSK DEQIWQSAVY YLCKEIYYND | 660 |
| FLQSKEAYKL FREGVKNLDK NDINNOKAAD SFKQAVVYYG KAIGNATLSQ VCQAIMTEYN | 720 |
| RONNDGLKKK SAYAEKONSN KYKHYPLFLK QVLQSAFWEY LDENKEIYGF ISAQIHKSNV | 780 |
| EIKAEDFIAN YSSQQYKKLV DKVKKTPELQ KWYTLGRLIN PROANQFLGS IRNYVQFVKD | 840 |
| IQRRAKENGN PIRNYYEVLE SDSIIKILEM CTKLNGTTSN DIHDYFRDED EYAEYISQFV | 900 |
| NFGDVHSGAA LNAFCNSESE GKKNGIYYDG INPIVNRNWV LCKLYGSPDL ISKIISRVNE | 960 |
| NMIHDFHKQE DLIREYQIKG ICSNKKEQQD LRTFQVLKNR VELRDIVEYS EIINELYGQL | 1020 |
| IKWCYLRERD LMYFQLGFHY LCLNNASSKE ADYIKINVDD RNISGAILYQ IAAMYINGLP | 1080 |
| VYYKKDDMYV ALKSGKKASD ELNSNEQTSK KINYFLKYGN NILGDKKDQL YLAGLELFEN | 1140 |
| VAEHENIIIF RNEIDHFHYF YDRDRSMLDL YSEVFDRFFT YDMKLRKNVV NMLYNILLDH | 1200 |
| NIVSSFVFET GEKKVGRGDS EVIKPSAKIR LRANNGVSSD VFTYKVGSKD ELKIATLPAK | 1260 |
| NEEFLLNVAR LIYYPDMEAV SENMVREGVV KVEKSNDKKG KISRGSNTRS SNQSKYNNKS | 1320 |
| KNRMNYSMGS IFEKMDLKFD | 1340 |
| (1125 in PCT/US2017/017255) | |
| SEQ ID NO: 45 | |
| MKISKVREEN RGAKLTVNAK TAVVSENRSQ EGILYNDPSR YGKSRKNDED RDRYIESRLK | 60 |
| SSGKLYRIFN EDKNKRETDE LOWFLSEIVK KINRRNGLVL SDMLSVDDRA FEKAFEKYAE | 120 |
| LSYTNRRNKV SGSPAFETCG VDAATAERLK GIISETNFIN RIKNNIDNKV SEDIIDRIIA | 180 |
| KYLKKSLCRE RVKRGLKKLL MNAFDLPYSD PDIDVQRDFI DYVLEDFYHV RAKSQVSRSI | 240 |
| KNMNMPVQPE GDGKFAITVS KGGTESGNKR SAEKEAFKKF LSDYASLDER VRDDMLRRMR | 300 |
| RLVVLYFYGS DDSKLSDVNE KFDVWEDHAA RRVDNREFIK LPLENKLANG KTDKDAERIR | 360 |
| KNTVKELYRN QNIGCYRQAV KAVEEDNNGR YFDDKMLNMF FIHRIEYGVE KIYANLKQVT | 420 |
| EFKARTGYLS EKIWKDLINY ISIKYIAMGK AVYNYAMDEL NASDKKEIEL GKISEEYLSG | 480 |
| ISSFDYELIK AEEMLORETA VYVAFAARHL SSQTVELDSE NSDFLLLKPK GTMDKNDKNK | 540 |
| LASNNILNFL KDKETLRDTI LQYFGGHSLW TDFPFDKYLA GGKDDVDFLT DLKDVIYSMR | 600 |
| NDSFHYATEN HNNGKWNKEL ISAMFEHETE RMTVVMKDKF YSNNLPMFYK NDDLKKLLID | 660 |
| LYKDNVERAS QVPSFNKVFV RKNFPALVRD KDNLGIELDL KADADKGENE LKFYNALYYM | 720 |
| FKEIYYNAFL NDKNVRERFI TKATKVADNY DRNKERNLKD RIKSAGSDEK KKLREQLQNY | 780 |
| IAENDFGQRI KNIVQVNPDY TLAQICQLIM TEYNQQNNGC MQKKSAARKD INKDSYQHYK | 840 |
| MLLLVNLRKA FLEFIKENYA FVLKPYKHDL CDKADFVPDF AKYVKPYAGL ISRVAGSSEL | 900 |
| QKWYIVSRFL SPAQANHMLG FLHSYKQYVW DIYRRASETG TEINHSIAED KIAGVDITDV | 960 |
| DAVIDLSVKL CGTISSEISD YFKDDEVYAE YISSYLDFEY DGGNYKDSLN RFCNSDAVND | 1020 |
| QKVALYYDGE HPKLNRNIIL SKLYGERRFL EKITDRVSRS DIVEYYKLKK ETSQYQTKGI | 1080 |
| FDSEDEQKNI KKFQEMKNIV EFRDLMDYSE IADELQGQLI NWIYLRERDL MNFQLGYHYA | 1140 |
| CLNNDSNKQA TYVTLDYQGK KNRKINGAIL YQICAMYING LPLYYVDKDS SEWTVSDGKE | 1200 |
| STGAKIGEFY RYAKSFENTS DCYASGLEIF ENISEHDNIT ELRNYIEHFR YYSSFDRSFL | 1260 |
| GIYSEVFDRF FTYDLKYRKN VPTILYNILL QHEVNVRFEF VSGKKMIGID KKDRKIAKEK | 1320 |
| ECARITIREK NGVYSEQFTY KLKNGTVYVD ARDKRYLQSI IRLLFYPEKV NMDEMIEVKE | 1380 |
| KKKPSDNNTG KGYSKRDRQQ DRKEYDKYKE KKKKEGNFLS GMGGNINWDE INAQLKN | 1437 |
| (1126 in PCT/US2017/017255) | |
| SEQ ID NO: 46 | |
| MKFSKVDHTR SAVGIQKATD SVHGMLYTDP KKQEVNDLDK RFDQLNVKAK RLYNVFNQSK | 60 |
| AEEDDDEKRF GKVVKKLNRE LKDLLFHREV SRYNSIGNAK YNYYGIKSNP EEIVSNLGMV | 120 |
| ESLKGERDPQ KVISKLLLYY LRKGLKPGTD GLRMILEASC GLRKLSGDEK ELKVFLQTLD | 180 |
| EDFEKKTFKK NLIRSIENQN MAVQPSNEGD PIIGITQGRF NSQKNEEKSA IERMMSMYAD | 240 |
| LNEDHREDVL RKLRRLNVLY FNVDTEKTEE PTLPGEVDTN PVFEVWHDHE KGKENDRQFA | 300 |
| TFAKILTEDR ETRKKEKLAV KEALNDLKSA IRDHNIMAYR CSIKVTEQDK DGLFFEDQRI | 360 |
| NRFWIHHIES AVERILASIN PEKLYKLRIG YLGEKVWKDL LNYLSIKYIA VGKAVFHFAM | 420 |
| EDLGKTGQDI ELGKLSNSVS GGLTSFDYEQ IRADETLORO LSVEVAFAAN NLFRAVVGQT | 480 |
| GKKIEQSKSE ENEEDFLLWK AEKIAESIKK EGEGNTLKSI LQFFGGASSW DLNHFCAAYG | 540 |
| NESSALGYET KFADDLRKAI YSLRNETFHF TTLNKGSFDW NAKLIGDMFS HEAATGIAVE | 600 |
| RTRFYSNNLP MFYRESDLKR IMDHLYNTYH PRASQVPSFN SVFVRKNFRL FLSNTLNTNT | 660 |
| SFDTEVYQKW ESGVYYLFKE IYYNSFLPSG DAHHLFFEGL RRIRKEADNL PIVGKEAKKR | 720 |
| NAVQDFGRRC DELKNLSLSA ICQMIMTEYN EQNNGNRKVK STREDKRKPD IFQHYKMLLL | 780 |
| RTLQEAFAIY IRREEFKFIF DLPKTLYVMK PVEEFLPNWK SGMFDSLVER VKQSPDLQRW | 840 |
| YVLCKFLNGR LLNOLSGVIR SYIQFAGDIQ RRAKANHNRL YMDNTQRVEY YSNVLEVVDF | 900 |
| CIKGTSRFSN VFSDYFRDED AYADYLDNYL QFKDEKIAEV SSFAALKTFC NEEEVKAGIY | 960 |
| MDGENPVMQR NIVMAKLFGP DEVLKNVVPK VTREEIEEYY QLEKQIAPYR QNGYCKSEED | 1020 |
| QKKLLRFQRI KNRVEFQTIT EFSEIINELL GOLISWSFLR ERDLLYFQLG FHYLCLHNDT | 1080 |
| EKPAEYKEIS REDGTVIRNA ILHQVAAMYV GGLPVYTLAD KKLAAFEKGE ADCKLSISKD | 1140 |
| TAGAGKKIKD FFRYSKYVLI KDRMLTDQNQ KYTIYLAGLE LFENTDEHDN ITDVRKYVDH | 1200 |
| FKYYATSDEN AMSILDLYSE IHDRFFTYDM KYQKNVANML ENILLRHFVL IRPEFFTGSK | 1260 |
| KVGEGKKITC KARAQIEIAE NGMRSEDFTY KLSDGKKNIS TCMIAARDQK YLNTVARLLY | 1320 |
| YPHEAKKSIV DTREKKNNKK TNRGDGTFNK QKGTARKEKD NGPREFNDTG FSNTPFAGFD | 1380 |
| PFRNS | 1385 |
| (1127 in PCT/US2017/017255) | |
| SEQ ID NO: 47 | |
| MKISKVDHTR MAVAKGNOHR RDEISGILYK DPTKTGSIDF DERFKKLNCS AKILYHVENG | 60 |
| IAEGSNKYKN IVDKVNNNLD RVLFTGKSYD RKSIIDIDTV LRNVEKINAF DRISTEEREQ | 120 |
| IIDDLLEIQL RKGLRKGKAG LREVLLIGAG VIVRTDKKQE IADFLEILDE DENKTNQAKN | 180 |
| IKLSIENQGL VVSPVSRGEE RIFDVSGAQK GKSSKKAQEK EALSAFLLDY ADLDKNVRFE | 240 |
| YLRKIRRLIN LYFYVKNDDV MSLTEIPAEV NLEKDFDIWR DHEQRKEENG DFVGCPDILL | 300 |
| ADRDVKKSNS KOVKIAERQL RESIREKNIK RYRFSIKTIE KDDGTYFFAN KQISVFWIHR | 360 |
| IENAVERILG SINDKKLYRL RLGYLGEKVW KDILNFLSIK YIAVGKAVEN FAMDDLQEKD | 420 |
| RDIEPGKISE NAVNGLTSFD YEQIKADEML QREVAVNVAF AANNLARVTV DIPQNGEKED | 480 |
| ILLWNKSDIK KYKKNSKKGI LKSILQFFGG ASTWNMKMFE IAYHDQPGDY EENYLYDIIQ | 540 |
| ITYSLRNKSF HFKTYDHGDK NWNRELIGKM IEHDAERVIS VEREKFHSNN LPMFYKDADL | 600 |
| KKILDLLYSD YAGRASQVPA FNTVLVRKNF PEFLRKDMGY KVHFNNPEVE NOWHSAVYYL | 660 |
| YKEIYYNLFL RDKEVKNLFY TSLKNIRSEV SDKKQKLASD DFASRCEEIE DRSLPEICQI | 720 |
| IMTEYNAQNF GNRKVKSQRV IEKNKDIFRH YKMLLIKTLA GAFSLYLKQE RFAFIGKATP | 780 |
| IPYETTDVKN FLPEWKSGMY ASFVEEIKNN LDLQEWYIVG RFLNGRMLNQ LAGSLRSYIQ | 840 |
| YAEDIERRAA ENRNKLFSKP DEKIEACKKA VRVLDLCIKI STRISAEFTD YFDSEDDYAD | 900 |
| YLEKYLKYQD DAIKELSGSS YAALDHFCNK DDLKFDIYVN AGQKPILQRN IVMAKLFGPD | 960 |
| NILSEVMEKV TESAIREYYD YLKKVSGYRV RGKCSTEKEQ EDLLKFORLK NAVEFRDVTE | 1020 |
| YAEVINELLG QLISWSYLRE RDLLYFQLGF HYMCLKNKSF KPAEYVDIRR NNGTIIHNAI | 1080 |
| LYQIVSMYIN GLDFYSCDKE GKTLKPIETG KGVGSKIGQF IKYSQYLYND PSYKLEIYNA | 1140 |
| GLEVFENIDE HDNITDLRKY VDHFKYYAYG NKMSLLDLYS EFFDRFFTYD MKYQKNVVNV | 1200 |
| LENILLRHFV IFYPKFGSGK KDVGIRDCKK ERAQIEISEQ SLTSEDFMFK LDDKAGEEAK | 1260 |
| KFPARDERYL QTIAKLLYYP NEIEDMNREM KKGETINKKV QFNRKKKITR KQKNNSSNEV | 1320 |
| LSSTMGYLFK NIKL | 1334 |
| (1128 in PCT/US2017/017255) | |
| Carnobacterium gallinarum | |
| SEQ ID NO: 48 | |
| MRITKVKIKL DNKLYQVTMQ KEEKYGTLKL NEESRKSTAE ILRLKKASFN KSFHSKTINS | 60 |
| QKENKNATIK KNGDYISQIF EKLVGVDTNK NIRKPKMSLT DLKDLPKKDL ALFIKRKFKN | 120 |
| DDIVEIKNLD LISLFYNALQ KVPGEHFTDE SWADFCQEMM PYREYKNKFI ERKIILLANS | 180 |
| IEQNKGFSIN PETFSKRKRV LHQWAIEVQE RGDFSILDEK LSKLAEIYNF KKMCKRVQDE | 240 |
| LNDLEKSMKK GKNPEKEKEA YKKQKNFKIK TIWKDYPYKT HIGLIEKIKE NEELNOFNIE | 300 |
| IGKYFEHYFP IKKERCTEDE PYYLNSETIA TTVNYQLKNA LISYLMQIGK YKQFGLENQV | 360 |
| LDSKKLQEIG IYEGFQTKFM DACVFATSSL KNIIEPMRSG DILGKREFKE AIATSSFVNY | 420 |
| HHFFPYFPFE LKGMKDRESE LIPFGEQTEA KOMQNIWALR GSVQQIRNEI FHSFDKNQKF | 480 |
| NLPQLDKSNF EFDASENSTG KSQSYIETDY KFLFEAEKNQ LEQFFIERIK SSGALEYYPL | 540 |
| KSLEKLFAKK EMKFSLGSQV VAFAPSYKKL VKKGHSYQTA TEGTANYLGL SYYNRYELKE | 600 |
| ESFQAQYYLL KLIYQYVFLP NFSQGNSPAF RETVKAILRI NKDEARKKMK KNKKFLRKYA | 660 |
| FEQVREMEFK ETPDQYMSYL QSEMREEKVR KAEKNDKGFE KNITMNFEKL LMQIFVKGFD | 720 |
| VFLTTFAGKE LLLSSEEKVI KETEISLSKK INEREKTLKA SIQVEHQLVA TNSAISYWLF | 780 |
| CKLLDSRHLN ELRNEMIKFK QSRIKFNHTQ HAELIQNLLP IVELTILSND YDEKNDSQNV | 840 |
| DVSAYFEDKS LYETAPYVOT DDRTRVSFRP ILKLEKYHTK SLIEALLKDN PQFRVAATDI | 900 |
| QEWMHKREEI GELVEKRKNL HTEWAEGQQT LGAEKREEYR DYCKKIDREN WKANKVTLTY | 960 |
| LSQLHYLITD LLGRMVGFSA LFERDLVYFS RSFSELGGET YHISDYKNLS GVLRLNAEVK | 1020 |
| PIKIKNIKVI DNEENPYKGN EPEVKPFLDR LHAYLENVIG IKAVHGKIRN QTAHLSVLQL | 1080 |
| ELSMIESMNN LRDLMAYDRK LKNAVTKSMI KILDKHGMIL KLKIDENHKN FEIESLIPKE | 1140 |
| IIHLKDKAIK TNOVSEEYCQ LVLALLTTNP GNQLN | 1175 |
| (1129 in PCT/US2017/017255) | |
| Paludibacter propionicigenes | |
| SEQ ID NO: 49 | |
| MRVSKVKVKD GGKDKMVLVH RKTTGAQLVY SGQPVSNETS NILPEKKRQS FDLSTLNKTI | 60 |
| IKFDTAKKQK LNVDQYKIVE KIFKYPKQEL PKQIKAEEIL PFLNHKFQEP VKYWKNGKEE | 120 |
| SFNLTLLIVE AVQAQDKRKL QPYYDWKTWY IQTKSDLLKK SIENNRIDLT ENLSKRKKAL | 180 |
| LAWETEFTAS GSIDLTHYHK VYMTDVLCKM LQDVKPLTDD KGKINTNAYH RGLKKALQNH | 240 |
| QPAIFGTREV PNEANRADNO LSTYHLEVVK YLEHYFPIKT SKRRNTADDI AHYLKAQTLK | 300 |
| TTIEKQLVNA IRANIIQQGK TNHHELKADT TSNDLIRIKT NEAFVLNLTG TCAFAANNIR | 360 |
| NMVDNEQTND ILGKGDFIKS LLKDNTNSQL YSFFFGEGLS TNKAEKETQL WGIRGAVQQI | 420 |
| RNNVNHYKKD ALKTVFNISN FENPTITDPK QQTNYADTIY KARFINELEK IPEAFAQQLK | 480 |
| TGGAVSYYTI ENLKSLLTTF QFSLCRSTIP FAPGFKKVEN GGINYQNAKQ DESFYELMLE | 540 |
| QYLRKENFAE ESYNARYFML KLIYNNLFLP GFTTDRKAFA DSVGFVQMQN KKQAEKVNPR | 600 |
| KKEAYAFEAV RPMTAADSIA DYMAYVQSEL MQEQNKKEEK VAEETRINFE KFVLQVFIKG | 660 |
| FDSFLRAKEF DFVOMPOPOL TATASNQQKA DKLNQLEASI TADCKLTPQY AKADDATHIA | 720 |
| FYVFCKLLDA AHLSNLRNEL IKFRESVNEF KFHHLLEIIE ICLLSADVVP TDYRDLYSSE | 780 |
| ADCLARLRPF IEQGADITNW SDLFVQSDKH SPVIHANIEL SVKYGTTKLL EQIINKDTQF | 840 |
| KTTEANFTAW NTAQKSIEQL IKQREDHHEQ WVKAKNADDK EKQERKREKS NFAQKFIEKH | 900 |
| GDDYLDICDY INTYNWLDNK MHFVHLNRLH GLTIELLGRM AGFVALFDRD FOFFDEQQIA | 960 |
| DEFKLHGFVN LHSIDKKLNE VPTKKIKEIY DIRNKIIQIN GNKINESVRA NLIQFISSKR | 1020 |
| NYYNNAFLHV SNDEIKEKOM YDIRNHIAHF NYLTKDAADF SLIDLINELR ELLHYDRKLK | 1080 |
| NAVSKAFIDL FDKHGMILKL KLNADHKLKV ESLEPKKIYH LGSSAKDKPE YQYCTNQVMM | 1140 |
| AYCNMCRSLL EMKK | 1154 |
| (1130 in PCT/US2017/017255) | |
| Listeria seeligeri | |
| SEQ ID NO: 50 | |
| MWISIKTLIH HLGVLFFCDY MYNRREKKII EVKTMRITKV EVDRKKVLIS RDKNGGKLVY | 60 |
| ENEMQDNTEQ IMHHKKSSFY KSVVNKTICR PEQKQMKKLV HGLLQENSQE KIKVSDVTKL | 120 |
| NISNFLNHRF KKSLYYFPEN SPDKSEEYRI EINLSQLLED SLKKQQGTFI CWESFSKDME | 180 |
| LYINWAENYI SSKTKLIKKS IRNNRIQSTE SRSGQLMDRY MKDILNKNKP FDIQSVSEKY | 240 |
| QLEKLTSALK ATFKEAKKND KEINYKLKST LONHERQIIE ELKENSELNQ FNIEIRKHLE | 300 |
| TYFPIKKTNR KVGDIRNLEI GEIQKIVNHR LKNKIVQRIL QEGKLASYEI ESTVNSNSLQ | 360 |
| KIKIEEAFAL KFINACLFAS NNLRNMVYPV CKKDILMIGE FKNSFKEIKH KKFIRQWSQF | 420 |
| FSQEITVDDI ELASWGLRGA IAPIRNEIIH LKKHSWKKFF NNPTFKVKKS KIINGKTKDV | 480 |
| TSEFLYKETL FKDYFYSELD SVPELIINKM ESSKILDYYS SDOLNQVFTI PNFELSLLTS | 540 |
| AVPFAPSFKR VYLKGFDYQN QDEAQPDYNL KLNIYNEKAF NSEAFQAQYS LFKMVYYQVF | 600 |
| LPQFTTNNDL FKSSVDFILT LNKERKGYAK AFQDIRKMNK DEKPSEYMSY IQSQLMLYQK | 660 |
| KQEEKEKINH FEKFINQVFI KGFNSFIEKN RLTYICHPTK NTVPENDNIE IPFHTDMDDS | 720 |
| NIAFWLMCKL LDAKQLSELR NEMIKFSCSL QSTEEISTFT KAREVIGLAL LNGEKGCNDW | 780 |
| KELFDDKEAW KKNMSLYVSE ELLQSLPYTQ EDGQTPVINR SIDLVKKYGT ETILEKLESS | 840 |
| SDDYKVSAKD IAKLHEYDVT EKIAQQESLH KOWIEKPGLA RDSAWTKKYQ NVINDISNYQ | 900 |
| WAKTKVELTQ VRHLHQLTID LLSRLAGYMS IADRDFQFSS NYILERENSE YRVTSWILLS | 960 |
| ENKNKNKYND YELYNLKNAS IKVSSKNDPQ LKVDLKQLRL TLEYLELFDN RLKEKRNNIS | 1020 |
| HFNYLNGQLG NSILELFDDA RDVLSYDRKL KNAVSKSLKE ILSSHGMEVT FKPLYQTNHH | 1080 |
| LKIDKLQPKK IHHLGEKSTV SSNQVSNEYC QLVRTLLTMK | 1120 |
| (1131 in PCT/US2017/017255) | |
| Listeria newyorkensis | |
| SEQ ID NO: 51 | |
| MKITKMRVDG RTIVMERTSK EGQLGYEGID GNKTTEIIFD KKKESFYKSI LNKTVRKPDE | 60 |
| KEKNRRKQAI NKAINKEITE LMLAVLHQEV PSQKLHNLKS LNTESLTKLF KPKFQNMISY | 120 |
| PPSKGAEHVQ FCLTDIAVPA IRDLDEIKPD WGIFFEKLKP YTDWAESYIH YKQTTIQKSI | 180 |
| EQNKIQSPDS PRKLVLQKYV TAFLNGEPLG LDLVAKKYKL ADLAESFKLV DLNEDKSANY | 240 |
| KIKACLQQHQ RNILDELKED PELNQYGIEV KKYIQRYFPI KRAPNRSKHA RADFLKKELI | 300 |
| ESTVEQQFKN AVYHYVLEQG KMEAYELTDP KTKDLQDIRS GEAFSFKFIN ACAFASNNLK | 360 |
| MILNPECEKD ILGKGNFKKN LPNSTTRSDV VKKMIPFFSD ELQNVNFDEA IWAIRGSIQQ | 420 |
| IRNEVYHCKK HSWKSILKIK GFEFEPNNMK YADSDMQKLM DKDIAKIPEF IEEKLKSSGV | 480 |
| VRFYRHDELQ SIWEMKOGFS LLTTNAPFVP SFKRVYAKGH DYQTSKNRYY NLDLTTFDIL | 540 |
| EYGEEDFRAR YFLTKLVYYQ QFMPWFTADN NAFRDAANFV LRLNKNRQQD AKAFINIREV | 600 |
| EEGEMPRDYM GYVQGQIAIH EDSIEDTPNH FEKFISQVFI KGFDRHMRSA NLKFIKNPRN | 660 |
| QGLEQSEIEE MSFDIKVEPS FLKNKDDYIA FWIFCKMLDA RHLSELRNEM IKYDGHLTGE | 720 |
| QEIIGLALLG VDSRENDWKQ FFSSEREYEK IMKGYVVEEL YQREPYRQSD GKTPILFRGV | 780 |
| EQARKYGTET VIQRLFDANP EFKVSKCNLA EWERQKETIE ETIKRRKELH NEWAKNPKKP | 840 |
| QNNAFFKEYK ECCDAIDAYN WHKNKTTLAY VNELHHLLIE ILGRYVGYVA IADRDFQCMA | 900 |
| NQYFKHSGIT ERVEYWGDNR LKSIKKLDTF LKKEGLFVSE KNARNHIAHL NYLSLKSECT | 960 |
| LLYLSERLRE IFKYDRKLKN AVSKSLIDIL DRHGMSVVFA NLKENKHRLV IKSLEPKKLR | 1020 |
| HLGGKKIDGG YIETNQVSEE YCGIVKRLLE M | 1051 |
| (1132 in PCT/US2017/017255) | |
| SEQ ID NO: 52 | |
| MYMKITKIDG VSHYKKQDKG ILKKKWKDLD ERKOREKIEA RYNKQIESKI YKEFFRLKNK | 60 |
| KRIEKEEDON IKSLYFFIKE LYLNEKNEEW ELKNINLEIL DDKERVIKGY KFKEDVYFFK | 120 |
| EGYKEYYLRI LFNNLIEKVQ NENREKVRKN KEFLDLKEIF KKYKNRKIDL LLKSINNNKI | 180 |
| NLEYKKENVN EEIYGINPTN DREMTFYELL KEIIEKKDEQ KSILEEKLDN FDITNFLENI | 240 |
| EKIFNEETEI NIIKGKVLNE LREYIKEKEE NNSDNKLKQI YNLELKKYIE NNFSYKKQKS | 300 |
| KSKNGKNDYL YLNFLKKIMF IEEVDEKKEI NKEKFKNKIN SNFKNLFVQH ILDYGKLLYY | 360 |
| KENDEYIKNT GQLETKDLEY IKTKETLIRK MAVLVSFAAN SYYNLFGRVS GDILGTEVVK | 420 |
| SSKTNVIKVG SHIFKEKMLN YFFDFEIFDA NKIVEILESI SYSIYNVRNG VGHFNKLILG | 480 |
| KYKKKDINTN KRIEEDLNNN EEIKGYFIKK RGEIERKVKE KFLSNNLQYY YSKEKIENYF | 540 |
| EVYEFEILKR KIPFAPNFKR IIKKGEDLEN NKNNKKYEYF KNFDKNSAEE KKEFLKTRNF | 600 |
| LLKELYYNNF YKEFLSKKEE FEKIVLEVKE EKKSRGNINN KKSGVSFQSI DDYDTKINIS | 660 |
| DYIASIHKKE MERVEKYNEE KOKDTAKYIR DFVEEIFLTG FINYLEKDKR LHFLKEEFSI | 720 |
| LCNNNNNVVD FNININEEKI KEFLKENDSK TLNLYLFFNM IDSKRISEFR NELVKYKQFT | 780 |
| KKRLDEEKEF LGIKIELYET LIEFVILTRE KLDTKKSEEI DAWLVDKLYV KDSNEYKEYE | 840 |
| EILKLFVDEK ILSSKEAPYY ATDNKTPILL SNFEKTRKYG TOSFLSEIQS NYKYSKVEKE | 900 |
| NIEDYNKKEE IEQKKKSNIE KLQDLKVELH KKWEQNKITE KEIEKYNNTT RKINEYNYLK | 960 |
| NKEELQNVYL LHEMLSDLLA RNVAFFNKWE RDFKFIVIAI KQFLRENDKE KVNEFLNPPD | 1020 |
| NSKGKKVYFS VSKYKNIVEN IDGIHKNEMN LIFLNNKFMN RKIDKMNCAI WVYFRNYIAH | 1080 |
| FLHLHTKNEK ISLISQMNLL IKLFSYDKKV QNHILKSTKT LLEKYNIQIN FEISNDKNEV | 1140 |
| FKYKIKNRLY SKKGKMLGKN NKFEILENEF LENVKAMLEY SE | 1182 |
| (1133 in PCT/US2017/017255) | |
| Rhodobacter capsulatus | |
| SEQ ID NO: 53 | |
| MQIGKVQGRT ISEFGDPAGG LKRKISTDGK NRKELPAHLS SDPKALIGQW ISGIDKIYRK | 60 |
| PDSRKSDGKA IHSPTPSKMQ FDARDDLGEA FWKLVSEAGL AQDSDYDQFK RRLHPYGDKF | 120 |
| QPADSGAKLK FEADPPEPQA FHGRWYGAMS KRGNDAKELA AALYEHLHVD EKRIDGQPKR | 180 |
| NPKTDKFAPG LVVARALGIE SSVLPRGMAR LARNWGEEEI QTYFVVDVAA SVKEVAKAAV | 240 |
| SAAQAFDPPR QVSGRSLSPK VGFALAEHLE RVTGSKRCSF DPAAGPSVLA LHDEVKKTYK | 300 |
| RLCARGKNAA RAFPADKTEL LALMRHTHEN RVRNQMVRMG RVSEYRGQQA GDLAQSHYWT | 360 |
| SAGQTEIKES EIFVRLWVGA FALAGRSMKA WIDPMGKIVN TEKNDRDLTA AVNIRQVISN | 420 |
| KEMVAEAMAR RGIYFGETPE LDRLGAEGNE GFVFALLRYL RGCRNQTFHL GARAGFLKEI | 480 |
| RKELEKTRWG KAKEAEHVVL TDKTVAAIRA IIDNDAKALG ARLLADLSGA FVAHYASKEH | 540 |
| FSTLYSEIVK AVKDAPEVSS GLPRLKLLLK RADGVRGYVH GLRDTRKHAF ATKLPPPPAP | 600 |
| RELDDPATKA RYIALLRLYD GPFRAYASGI TGTALAGPAA RAKEAATALA QSVNVTKAYS | 660 |
| DVMEGRSSRL RPPNDGETLR EYLSALTGET ATEFRVQIGY ESDSENARKQ AEFIENYRRD | 720 |
| MLAFMFEDYI RAKGFDWILK IEPGATAMTR APVLPEPIDT RGQYEHWQAA LYLVMHFVPA | 780 |
| SDVSNLLHQL RKWEALQGKY ELVQDGDATD QADARREALD LVKRFRDVLV LFLKTGEARF | 840 |
| EGRAAPFDLK PFRALFANPA TFDRLFMATP TTARPAEDDP EGDGASEPEL RVARTLRGLR | 900 |
| QIARYNHMAV LSDLFAKHKV RDEEVARLAE IEDETQEKSQ IVAAQELRTD LHDKVMKCHP | 960 |
| KTISPEERQS YAAAIKTIEE HRFLVGRVYL GDHLRLHRLM MDVIGRLIDY AGAYERDTGT | 1020 |
| FLINASKOLG AGADWAVTIA GAANTDARTO TRKDLAHFNV LDRADGTPDL TALVNRAREM | 1080 |
| MAYDRKRKNA VPRSILDMLA RLGLTLKWQM KDHLLQDATI TOAAIKHLDK VRLTVGGPAA | 1140 |
| VTEARFSQDY LOMVAAVFNG SVQNPKPRRR DDGDAWHKPP KPATAQSQPD QKPPNKAPSA | 1200 |
| GSRLPPPQVG EVYEGVVVKV IDTGSLGFLA VEGVAGNIGL HISRLRRIRE DAIIVGRRYR | 1260 |
| FRVEIYVPPK SNTSKLNAAD LVRID | 1285 |
| (1134 in PCT/US2017/017255) | |
| Leptotrichia buccalis | |
| SEQ ID NO: 54 | |
| MKVTKVGGIS HKKYTSEGRL VKSESEENRT DERLSALLNM RLDMYIKNPS STETKENQKR | 60 |
| IGKLKKFFSN KMVYLKDNTL SLKNGKKENI DREYSETDIL ESDVRDKKNF AVLKKIYLNE | 120 |
| NVNSEELEVF RNDIKKKLNK INSLKYSFEK NKANYQKINE NNIEKVEGKS KRNIIYDYYR | 180 |
| ESAKRDAYVS NVKEAFDKLY KEEDIAKLVL EIENLTKLEK YKIREFYHEI IGRKNDKENF | 240 |
| AKIIYEEIQN VNNMKELIEK VPDMSELKKS QVFYKYYLDK EELNDKNIKY AFCHFVEIEM | 300 |
| SQLLKNYVYK RLSNISNDKI KRIFEYQNLK KLIENKLINK LDTYVRNCGK YNYYLQDGEI | 360 |
| ATSDFIARNR QNEAFLRNII GVSSVAYFSL RNILETENEN DITGRMRGKT VKNNKGEEKY | 420 |
| VSGEVDKIYN ENKKNEVKEN LKMFYSYDEN MDNKNEIEDF FANIDEAISS IRHGIVHFNL | 480 |
| ELEGKDIFAF KNIAPSEISK KMFQNEINEK KLKLKIFRQL NSANVFRYLE KYKILNYLKR | 540 |
| TRFEFVNKNI PFVPSFTKLY SRIDDLKNSL GIYWKTPKTN DDNKTKEIID AQIYLLKNIY | 600 |
| YGEFLNYFMS NNGNFFEISK EIIELNKNDK RNLKTGFYKL QKFEDIQEKI PKEYLANIQS | 660 |
| LYMINAGNQD EEEKDTYIDF IQKIFLKGFM TYLANNGRLS LIYIGSDEET NTSLAEKKQE | 720 |
| FDKFLKKYEQ NNNIKIPYEI NEFLREIKLG NILKYTERLN MFYLILKLLN HKELTNLKGS | 780 |
| LEKYQSANKE EAFSDQLELI NLLNLDNNRV TEDFELEADE IGKFLDFNGN KVKDNKELKK | 840 |
| FDTNKIYFDG ENIIKHRAFY NIKKYGMLNL LEKIADKAGY KISIEELKKY SNKKNEIEKN | 900 |
| HKMQENLHRK YARPRKDEKF TDEDYESYKQ AIENIEEYTH LKNKVEFNEL NLLQGLLLRI | 960 |
| LHRLVGYTSI WERDLRFRLK GEFPENQYIE EIFNFENKKN VKYKGGQIVE KYIKFYKELH | 1020 |
| QNDEVKINKY SSANIKVLKQ EKKDLYIRNY IAHFNYIPHA EISLLEVLEN LRKLLSYDRK | 1080 |
| LKNAVMKSVV DILKEYGFVA TFKIGADKKI GIQTLESEKI VHLKNLKKKK LMTDRNSEEL | 1140 |
| CKLVKIMFEY KMEEKKSEN | 1159 |
| (1135 in PCT/US2017/017255) | |
| Leptotrichia shahii | |
| SEQ ID NO: 55 | |
| MGNLFGHKRW YEVRDKKDFK IKRKVKVKRN YDGNKYILNI NENNNKEKID NNKFIRKYIN | 60 |
| YKKNDNILKE FTRKFHAGNI LFKLKGKEGI IRIENNDDFL ETEEVVLYIE AYGKSEKLKA | 120 |
| LGITKKKIID EAIRQGITKD DKKIEIKROE NEEEIEIDIR DEYTNKTLND CSIILRIIEN | 180 |
| DELETKKSIY EIFKNINMSL YKIIEKIIEN ETEKVFENRY YEEHLREKLL KDDKIDVILT | 240 |
| NFMEIREKIK SNLEILGFVK FYLNVGGDKK KSKNKKMLVE KILNINVDLT VEDIADFVIK | 300 |
| ELEFWNITKR IEKVKKVNNE FLEKRRNRTY IKSYVLLDKH EKFKIERENK KDKIVKFFVE | 360 |
| NIKNNSIKEK IEKILAEFKI DELIKKLEKE LKKGNCDTEI FGIFKKHYKV NFDSKKFSKK | 420 |
| SDEEKELYKI IYRYLKGRIE KILVNEQKVR LKKMEKIEIE KILNESILSE KILKRVKQYT | 480 |
| LEHIMYLGKL RHNDIDMTTV NTDDFSRLHA KEELDLELIT FFASTNMELN KIFSRENINN | 540 |
| DENIDFFGGD REKNYVLDKK ILNSKIKIIR DLDFIDNKNN ITNNFIRKFT KIGTNERNRI | 600 |
| LHAISKERDL QGTQDDYNKV INIIQNLKIS DEEVSKALNL DVVFKDKKNI ITKINDIKIS | 660 |
| EENNNDIKYL PSFSKVLPEI LNLYRNNPKN EPFDTIETEK IVLNALIYVN KELYKKLILE | 720 |
| DDLEENESKN IFLQELKKTL GNIDEIDENI IENYYKNAQI SASKGNNKAI KKYQKKVIEC | 780 |
| YIGYLRKNYE ELFDFSDFKM NIQEIKKOIK DINDNKTYER ITVKTSDKTI VINDDFEYII | 840 |
| SIFALLNSNA VINKIRNRFF ATSVWLNTSE YQNIIDILDE IMQLNTLRNE CITENWNLNL | 900 |
| EEFIQKMKEI EKDFDDFKIQ TKKEIFNNYY EDIKNNILTE FKDDINGCDV LEKKLEKIVI | 960 |
| FDDETKFEID KKSNILQDEQ RKLSNINKKD LKKKVDQYIK DKDQEIKSKI LCRIIFNSDF | 1020 |
| LKKYKKEIDN LIEDMESENE NKFQEIYYPK ERKNELYIYK KNLFLNIGNP NFDKIYGLIS | 1080 |
| NDIKMADAKF LFNIDGKNIR KNKISEIDAI LKNLNDKLNG YSKEYKEKYI KKLKENDDFF | 1140 |
| AKNIQNKNYK SFEKDYNRVS EYKKIRDLVE FNYLNKIESY LIDINWKLAI QMARFERDMH | 1200 |
| YIVNGLRELG IIKLSGYNTG ISRAYPKRNG SDGFYTTTAY YKFFDEESYK KFEKICYGFG | 1260 |
| IDLSENSEIN KPENESIRNY ISHFYIVRNP FADYSIAEQI DRVSNLLSYS TRYNNSTYAS | 1320 |
| VFEVFKKDVN LDYDELKKKF KLIGNNDILE RLMKPKKVSV LELESYNSDY IKNLIIELLT | 1380 |
| KIENTNDTL | 1389 |

1. A synthetic exosome capable of delivering a therapeutic moiety across the blood brain barrier into the central nervous system (CNS), said synthetic exosome comprising:
a liposome formed from a lipid bilayer, where said lipid bilayer comprises:
one or more phospholipids selected from the group consisting of phosphate lipids, phosphoglycerol lipids, phosphocholine lipids, and phosphoethanolamine lipids where the lipid carbon chain ranges from 3 to 24 carbon atoms;
cholesterol, a cholesterol derivative, or a phytosterol; and
a non-ionic surfactant;
wherein said lipid bilayer does not contain an alcohol; and
said liposome ranges in size from up to about 500 nm in diameter.

2-3. (canceled)

4. The synthetic exosome of claim 1, wherein:
said synthetic exosome is capable of crossing the blood brain barrier without substantially leaking said therapeutic moiety; and/or
said exosome is capable of crossing the blood/brain barrier (BBB) and delivering a therapeutic moiety contained therein to the central nervous system without substantial loss of said therapeutic moiety; and/or
said exosome is capable of crossing the blood/brain barrier (BBB) and delivering a therapeutic moiety contained therein to the central nervous system without losing more than about 40%, or without losing more than 30%, or without losing more than 20%, or without losing more than 10%, or without losing more than 5%, or without losing more than 3%, or without losing more than 1% of a therapeutic moiety contained therein.

5. The synthetic exosome of claim 1, wherein said lipid bilayer consists of said one or more phospholipids, said cholesterol or cholesterol derivative or a phytosterol; and said non-ionic surfactant.

6-9. (canceled)

10. The synthetic exosome of claim 1, wherein:
said bilayer does not contain glutathione-maleimide-PEG2000-distearoyl phosphatidyl ethanolamine; and/or
said exosome is not a transferosome; and/or
said exosome is not an ethosome.

11-12. (canceled)

13. The synthetic exosome of claim 1, wherein:
the molar ratio of total phospholipid to cholesterol, cholesterol, or phytosterol ranges from about 6-10 moles of total phospholipid to about 1-3 moles of cholesterol; and/or
the amount of surfactant ranges from about 1%, or from about 3%, or from about 5%, or from about 8% up to about 18%, or up to about 15%, or up to about 13%, or up to about 10% (wt/wt).

14. (canceled)

15. The synthetic exosome of claim 1, wherein said surfactant comprise one or more surfactants selected from the group consisting of Span 80, Tween 20, BRIJ® 76 (stearyl poly(10)oxy ethylene ether), BRIJ® 78 (stearyl poly(20)oxyethylene ether), BRIJ® 96 (oleyl poly(10)oxy ethylene ether), and BRIJ® 721 (stearyl poly (21) oxyethylene ether).

16. (canceled)

17. The synthetic exosome of claim 15, wherein the lipid bilayer comprises about 10% to about 20%, or about 15% Span 80 by weight.

18. The synthetic exosome of claim 1, wherein:
said cholesterol, cholesterol derivative, or phytosterol comprises or consists of cholesterol; or
said cholesterol, cholesterol derivative, or phytosterol comprises or consists of a cholesterol derivative selected from the group consisting of cholesterol hemisuccinate, lysine-based cholesterol (CHLYS), 20-hydroxychloesterol, 22-hydroxycholesterol, 24-hydroxycholesterol, 25-hydroxy cholesterol, 27-hydroxycholesterol, cholesteryl succinate, cholic succinate, cholic tri-succinate, lithocholic succinate, chenodesoxycholic bis-scuccinate, and Hederoside; or
said cholesterol, cholesterol derivative comprises or consists cholesterol hemisuccinate; or
said cholesterol, cholesterol derivative, or phytosterol comprises or consists of a phytosterol; or
said cholesterol, cholesterol derivative, or phytosterol comprises or consists of a phytosterol that comprises a 9,10-secosteroid; or
said cholesterol, cholesterol derivative, or phytosterol comprises or consists of a compound selected from the group consisting of vitamin D3, vitamin D2, calcipotriol; or
said cholesterol, cholesterol derivative, or phytosterol comprises or consists of a C-24 alkyl steroid; or
said cholesterol, cholesterol derivative, or phytosterol comprises or consists of a compound selected from the group consisting of stigmasterol, and β-sitosterol; or
said cholesterol, cholesterol derivative, or phytosterol comprises or consists of a pentacyclic steroid; or
said cholesterol, cholesterol derivative, or phytosterol comprises or consists of a pentacyclic steroid that comprises a compound selected from the group consisting of betulin, lupeol, ursolic acid, and oleanolic acid; or
said cholesterol, cholesterol derivative, or phytosterol is pegylated.

19-28. (canceled)

29. The synthetic exosome of claim 1, wherein:
said one or more phospholipids comprises one or more phospholipids selected from the group consisting of dihexanoyl-sn-glycero-3-phosphate (DHPA), didecanoyl-sn-glycero-3-phosphate (DDPA), distearoyl-sn-glycero-3-phosphate (DTPA), and dihexadecyl phosphate (DHP); and/or
said one or more phospholipids comprises one or more phosphoglycerol lipids selected from the group consisting of dihexanoyl-sn-glycero-3-phospho-(1′-rac-glycerol) (DHPG), dilauroyl-sn-glycero-3-phospho-(1′-rac-glycerol) (DLPG), and distearoyl-sn-glycero-3-phospho-(1′-rac-glycerol) (DTPG); and/or
said one or more phospholipids comprises one or more phosphocholine lipids selected from the group consisting of dipropionyl-sn-glycero-3-phosphocholine (PC), diheptanoyl-sn-glycero-3-phosphocholine (DHPC), dimyristoyl-sn-glycero-3-phosphocholine (DMPC), and dilignoceroyl-sn-glycero-3-phosphocholine (DGPC), and/or
said one or more phospholipids comprises one or more phosphoethanolamine lipids selected from the group consisting of sihexanoyl-sn-glycero-3-phosphoethanolamine (DHPE), and distearoyl-sn-glycero-3-phosphoethanolamine (DTPE); and/or
said one or more phospholipids comprises one or more phosphoethanolamine-PEG lipids selected from the group consisting of dipalmitoyl-sn-glycero-3-phospho(ethylene glycol) (DPPEG1), dimyristoyl-sn-glycero-3-phosphoethanolamine-N-[methoxy(polyethylene glycol)-350 (DMPEG350), distearoyl-sn-glycero-3-phosphoethanolamine-N-[methoxy(polyethylene glycol)-350] (DTPEG350), dimyristoyl-sn-glycero-3-phosphoethanolamine-N-[methoxy(polyethylene glycol)-550] (DMPEG550), and dimyristoyl-sn-glycero-3-phosphoethanolamine-N-[methoxy(polyethylene glycol)-100] (DMPEG1000); and/or
said one or more phospholipids comprises one or more phospholipids selected from the group consisting of dioleoyl-sn-glycero-3-phosphocholine (N-aminoethyl) (PC-NH₂), diphytanoyl-sn-glycero-3-phosphoethanolamine, dioleoyl-3-trimethylammonium-propane (DOTAP), distearoyl-3-trimethylammonium-propane (DSTAP), dimyristoyl-3-trimethylammonium-propane (DMTAP), and di-O-octadecyl-sn-glycero-3-phosphocholin (DOPC).

30-35. (canceled)

36. The synthetic exosome of claim 1, wherein said lipid bilayer comprises or consists of:
said surfactant; and 3:2:1 molar ratio (DHPA:DHP:CH), 1:5:1 molar ratio (DHPG:DHPA:CH), 2:5:1:2 molar ratio (DHPG:DHPA:PC-NH₂:CH), or 2:4:1:1:2 molar ratio (DHPG:DHPA:PC-NH₂:DMPEG350:CH) to provide synthetic exosomes having a zeta potential of about −20 mV or lower; or
said surfactant; and 2:2:1 molar ratio (DHPG:DHPC:CH), 4:4:1:2 molar ratio (DHPG:DHPA:PC-NH₂:CH), 2:2:1 molar ratio (DHPG:DHPA:CH), 4:4:1:1:2 molar ratio (DHPG:DHPA:PC-NH₂:DMPEG550:CH), 2:2:1 molar ratio (DHPG:DTPE:CH), 2:2:1 molar ratio (DHPG:DMTAP:CH), 4:4:1:2 molar ratio (DHPG:DMTAP:PC-NH₂:CH), or 4:4:1:1:2 molar ratio (DHPG:DMTAP:PC-NH₂:DMPEG550:CH) to provide synthetic exosomes having a zeta potential ranging from about −20 mV to about 20 mV, or
said surfactant; and 2:4:1 molar ratio (DHPC:DTPE:CH), 2:4:1 molar ratio (DHPC:DOTAP:CH), 2:4:1:2 molar ratio (DHPC:DMTAP:PC-NH₂:CH), or 2:4:1:1:2 molar ratio (DHPC:DMTAP:PC-NH₂:DMPEG350:CH) to provide synthetic exosomes having a zeta potential of about 20 mV or greater; or
said surfactant; and 4:2:1 molar ratio (DTPA:DHP:CH), 1:5:1 molar ratio (DTPG:DTPA:CH), 1:5:1:2 molar ratio (DTPG:DTPA:PC-NH₂:CH), or 1:4:1:1:2 molar ratio (DTPG:DTPA:PC-NH₂:DMPEG350:CH) to provide synthetic exosomes having a zeta potential of about −20 mV or lower; or
said surfactant; and 2:2:1 molar ratio (DTPG:DGPC:CH), 4:4:1:2 molar ratio (DTPG:DDPA:PC-NH₂:CH), 2:2:1 molar ratio (DTPG:DDPA:CH), 4:4:1:1:2 molar ratio (DTPG:DDPA:PC-NH₂:DMPEG550:CH), 2:2:1 molar ratio (DTPG:DTPE:CH), 2:2:1 molar ratio (DTPG:DMTAP:CH), 4:4:1:2 molar ratio (DTPG:DMTAP:PC-NH₂:CH), or 4:4:1:1:2 molar ratio (DTPG:DMTAP:PC-NH₂:DMPEG550:CH) to provide synthetic exosomes having a zeta potential ranging from about −20 mV to about 20 mV, or
said surfactant; and 2:4:1 molar ratio (DMPC:DTPE:CH), 2:4:1 molar ratio (DMPC:DOTAP:CH), 2:4:1:2 molar ratio (DMPC:DMTAP:PC-NH₂:CH), or 2:4:1:1:2 molar ratio (DMPC:DMTAP:PC-NH₂:DMPEG350:CH) to provide synthetic exosomes having a zeta potential of about 20 mV or greater;
wherein CH is cholesterol or a cholesterol derivative.

37-48. (canceled)

49. The synthetic exosome of claim 1, wherein:
a targeting moiety comprising or consisting of transferrin, or folic acid, or an amino acid, or insulin, or a low density lipoprotein receptor related protein 1 is attached to said exosome; or
a targeting moiety comprising or consisting of a blood brain barrier targeting antibody is attached to said exosome; or
said exosome is attached to an antibody or a ligand that binds to a moiety selected from the group consisting of a transferrin receptor, an insulin receptor, an insulin growth factor receptor (IGF1R), a low-density lipoprotein (LDL) receptor, basigin, Glut1, CD98hc, and TMEM30A(cdc50A); or
said exosome is attached to an antibody or a ligand that binds to a cell surface marker; or
said exosome is attached to an antibody or a ligand that binds to a cell surface marker that is a marker of neural or glial cells; or
said exosome is attached to an antibody or a ligand that binds to a cell surface marker selected from the group consisting of CD63, CD81, CD9, and CD171, and is incorporated in the lipid bilayer of said exosome; or
said one or more phospholipids is functionalized with a targeting moiety selected from the group consisting of transferrin, an amino acid, a blood brain barrier targeting antibody, insulin, folic acid, and low density lipoprotein receptor related protein 1.

50-54. (canceled)

55. The synthetic exosome of claim 1, wherein said exosome contains one or more therapeutic moieties, wherein:
said therapeutic moiety is selected from the group consisting of a protein, an antibody, an enzyme, a DNA encoding an inhibitory RNA, an inhibitory RNA or a micoRNA (miRNA), a nucleic acid encoding a CRISPR endonuclease and a guide RNA, a CRISPR endonuclease and a guide RNA, and a small organic molecule; or
said therapeutic moiety comprises an sAPPα protein; or
said therapeutic moiety comprises IDUA (e.g., for MPS1) or acid sphingomyelinase (ASM) for Niemann Pick disease; or
said therapeutic moiety comprises an antibody; or
said therapeutic moiety comprises an antibody selected from the group consisting of full-length immunoglobulins, Fab, Fab′, Fab′-SH, F(ab′)₂, Fv, Fv′, Fd, Fd′, scFv, hsFv fragments, single-chain antibodies, and cameloid antibodies; or
said therapeutic moiety comprises an antibody for the treatment of a neurodegenerative condition or for the treatment of a cancer; or
said therapeutic moiety comprises said antibody comprise an antibody for the treatment of a neurodegenerative condition selected from the group consisting of Alzheimer's disease, amyotrophic lateral sclerosis (ALS), Huntington's disease, and Parkinson's disease; or
said therapeutic moiety comprises an antibody that binds to a target selected from the group consisting of Aβ, mutant Aβ, tau, mutant tau, apoE, and α-synuclein; or
said therapeutic moiety comprises an antibody selected from the group consisting of AAB-003, Bapineuzumab, Ponezumab, RG7345, Solanezumab, GSK933776, JNJ-63733657, BIIB076, LY2599666, MEDI1314, SAR228810, BAN2401, BIIB092, C2B8E12, LY3002813, LY3303560, RO 7105705, Aducanumab, Crenezumab, PRX002 (prasinezumab), and Gantenerumab, or combinations thereof; or
said therapeutic moiety comprises an anti-tau antibody; or
said therapeutic moiety comprises an anti-tau antibody selected from the group consisting of BIIB092, ABBV-8E12, R07105705, LY3303560, RG7345, R06926496, JNJ63733657, and UCB0107; or
said therapeutic moiety comprises an anti-ApoE antibody; or
said therapeutic moiety comprises an antibody for the treatment of amyotrophic lateral sclerosis (ALS); or
said therapeutic moiety comprises an antibody that binds to a misfolded SOD1 species; or
said therapeutic moiety comprises an antibody for the treatment of Huntington's disease; or
said therapeutic moiety comprises an anti-SEMA4D antibody (e.g., VX15); or
said therapeutic moiety comprises an antibody for the treatment of Parkinson's disease; or
said therapeutic moiety comprises an anti-α-synuclein antibody (e.g., prasinezumab); or
said therapeutic moiety comprises an antibody for the treatment of a cancer; or
said therapeutic moiety comprises a checkpoint PD-1 blocker; or′
said therapeutic moiety comprises Keytuda for treatment of Gliomas and Brain cancer.

56-85. (canceled)

86. The synthetic exosome of claim 1, wherein said synthetic exosome contains an enzyme for enzyme replacement therapy (ERT).

87. The synthetic exosome of claim 1, wherein:
said synthetic exosome contains components of a CRISPR/Cas system for the treatment of Alzheimer's disease, Parkinson's disease, ALS, fragile X syndrome (FXS), Huntington disease, autosomal dominant spinocereberal ataxis (SCAs), spinal bulbar muscular atrophy (SBMA), correction of autosomal recessive genetic disorder that is caused by a deficiency in the expression or function of the Ataxia Telangiectasia Mutated (ATM) protein; and/or
said synthetic exosome contains a plasmid that encodes a class 2 CRISPR/Cas endonuclease and a guide RNA or a nucleic acid encoding a guide RNA, or said synthetic exosome contains a class 2 CRISPR/Cas endonuclease and a guide RNA or a nucleic acid encoding a guide RNA; and/or
said synthetic exosome contains' a Cas9 polypeptide and the corresponding CRISPR/Cas guide RNA is a Cas9 guide RNA; and/or
said synthetic exosome contains a type V or type VI CRISPR/Cas endonuclease; and/or
said synthetic exosome contains a class 2 CRISPR/Cas endonuclease selected from the group consisting of a Cpf1 polypeptide or a functional portion thereof, a C2c1 polypeptide or a functional portion thereof, a C2c3 polypeptide or a functional portion thereof, and a C2c2 polypeptide or a functional portion thereof; and/or
said synthetic exosome contains components of a CRISPR/Cas system are configured to produce insertions or deletions in ApoE4; and/or
said synthetic exosome contains components of a CRISPR/Cas system configured to replace ApoE4 with ApoE3 or ApoE2.

88-101. (canceled)

102. The synthetic exosome of claim 1, wherein:
said synthetic exosome contains an miRNA; and/or
said synthetic exosome contains an inhibitory RNA, or a nucleic acid encoding an inhibitory RNA; and/or
said exosome contains a DNA encoding an shRNA or an siRNA; and/or
said exosome contains an inhibitory RNA or a nucleic acid encoding an inhibitory RNA for the treatment of a neurodegenerative condition or a cancer; and/or
said exosome contains an inhibitory RNA or a nucleic acid encoding an inhibitory RNA for the treatment of a neurodegenerative condition selected from the group consisting of Alzheimer's disease, amyotrophic lateral sclerosis (ALS), Huntington's disease, and Parkinson's disease; and/or
said exosome contains an inhibitory RNA or a nucleic acid encoding an inhibitory RNA for the treatment of Alzheimer's disease; and/or
said exosome contains an inhibitory RNA or a nucleic acid encoding an inhibitory RNA wherein said inhibitory RNA inhibits expression of a target selected from the group consisting of a mutant APP (e.g., APPsw), and a mutant tau; and/or
said exosome contains an inhibitory RNA or a nucleic acid encoding an inhibitory RNA wherein said inhibitory RNA inhibits expression of a target selected from the group consisting of c-SCR, GGA3 adaptor protein, and acyl-coenzyme A cholesterol acyltransferase (ACAT-1).

103-109. (canceled)

110. A pharmaceutical formulation comprising:
a synthetic exosome according to claim 1; and
a pharmaceutically acceptable carrier.

111. A kit comprising:
a container containing a nanoscale synthetic exosome according to claim 1; and
instructional materials teaching the use of said synthetic exosome to mitigate one or more symptoms associated with a disease characterized by amyloid deposits in the brain, and/or the use of said composition in delaying or preventing the onset of one or more of said symptoms.

112. A method of reducing the risk, lessening the severity, or delaying the progression or onset of a disease characterized by beta-amyloid deposits in the brain of a mammal, said method comprising:
administering, or causing to be administered, to said mammal synthetic exosome according to claim 1, wherein said exosome contains an antibody for the treatment of a neurodegenerative condition selected from the group consisting of Alzheimer's disease, amyotrophic lateral sclerosis (ALS), Huntington's disease, and Parkinson's disease or components of a CRISPR/Cas system for the treatment of Alzheimer's disease, Parkinson's disease, ALS, fragile X syndrome (FXS), Huntington disease, autosomal dominant spinocereberal ataxis (SCAs), spinal bulbar muscular atrophy (SBMA), correction of autosomal recessive genetic disorder that is caused by a deficiency in the expression or function of the Ataxia Telangiectasia Mutated (ATM) protein in an amount sufficient to reducing the risk, lessen the severity, or delay the progression or onset of said disease.

113. A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, said method comprising:
administering, or causing to be administered, to said mammal a synthetic exosome according to claim 1, wherein said exosome contains an antibody for the treatment of a neurodegenerative condition selected from the group consisting of Alzheimer's disease, amyotrophic lateral sclerosis (ALS), Huntington's disease, and Parkinson's disease or components of a CRISPR/Cas system for the treatment of Alzheimer's disease, Parkinson's disease, ALS, fragile X syndrome (FXS), Huntington disease, autosomal dominant spinocereberal ataxis (SCAs), spinal bulbar muscular atrophy (SBMA), correction of autosomal recessive genetic disorder that is caused by a deficiency in the expression or function of the Ataxia Telangiectasia Mutated (ATM) protein, in an amount sufficient to promote the processing of amyloid precursor protein (APP) by the non-amyloidogenic pathway and/or sufficient to reduce sAPPβ.

114. A method of promoting the processing of amyloid precursor protein (APP) by the non-amyloidogenic pathway as characterized by increasing sAPPα and/or the sAPPα/Aβ42 ratio in a mammal, said method comprising:
administering, or causing to be administered, to said mammal a synthetic exosome according to claim 1, wherein said exosome contains an antibody for the treatment of a neurodegenerative condition selected from the group consisting of Alzheimer's disease, amyotrophic lateral sclerosis (ALS), Huntington's disease, and Parkinson's disease or components of a CRISPR/Cas system for the treatment of Alzheimer's disease, Parkinson's disease, ALS, fragile X syndrome (FXS), Huntington disease, autosomal dominant spinocereberal ataxis (SCAs), spinal bulbar muscular atrophy (SBMA), correction of autosomal recessive genetic disorder that is caused by a deficiency in the expression or function of the Ataxia Telangiectasia Mutated (ATM) protein, wherein said administering is in an amount sufficient to promote the processing of amyloid precursor protein (APP) by the non-amyloidogenic pathway and/or sufficient to reduce sAPPβ.

115. A method of delivering one or more therapeutic moieties into the brain of a mammal, said method comprising:
administering, or causing to be administered, to said mammal an effective amount of a synthetic exosome according to claim 1, wherein said exosome contains said one or more therapeutic moieties.

116. A method of treating a pathology in a mammal selected from the group consisting of Alzheimer's disease, Parkinson's disease, ALS, fragile X syndrome (FXS), Huntington disease, autosomal dominant spinocereberal ataxis (SCAs), spinal bulbar muscular atrophy (SBMA), an autosomal recessive genetic disorder that is caused by a deficiency in the expression or function of the Ataxia Telangiectasia Mutated (ATM) protein, said method comprising:
administering, or causing to be administered, to said mammal an effective amount of a synthetic exosome according to claim 1, wherein said exosome contains an sAPPα protein.

117. A microfluidic flow reactor for the synthesis of synthetic exosomes, said reactor comprising:
a central channel with two or more branch channels feeding said central channel and thereby forming a mixing junction, where the diameter of said central channel and branch channels and the angle provided between said central channel and branch channels are selected to maintain a backpressure of less than about 100 psi.

118. (canceled)

119. A method of making a synthetic exosome containing a therapeutic moiety, said method comprising:
combining the components of a lipid bilayer as recited in claim 1 and said therapeutic moiety in organic and aqueous phases in microchannels in a microfluidic flow reactor at a controlled flow ratio and pressure; and
collecting the resulting samples comprising synthetic exosomes containing said therapeutic moiety.
